(19) United States
(12) Patent Application Publication
(10) Pub. No.: US 2010/0047171 A1
(43) Pub. Date: Feb. 25, 2010

(54) Fusion Proteins That Contain Natural Junctions

(76) Inventor: Roland Beckmann, Vienna (AT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/087,924

(22) PCT Filed: Jan. 24, 2007
(86) PCT No.: PCT/GB2007/000227

§ 371 Date: Mar. 16, 2009

Related U.S. Application Data
(60) Provisional application No. 60/761,708, filed on Jan. 24, 2006.

(30) Foreign Application Priority Data
Dec. 5, 2006 (GB) PCT/GB06/04559

(51) Int. Cl. C07K 19/00; A61K 49/00; C12P 21/00; C12N 1/21; C12N 1/15; C12N 1/19; C07H 21/04; C07K 14/525; C07K 14/435; C07K 14/705; C07K 16/46; C07K 1/00

(52) U.S. Cl. 424/9.1; 435/69.7; 435/252.3; 435/252.31; 435/252.33; 435/254.11; 435/254.2; 530/350; 530/391.1; 536/23.4

(57) ABSTRACT

A method for preparing recombinant fusion proteins that comprise at least one natural junction is described. Fusion proteins that contain at least one natural junction have reduced potential for immunogenicity, improved stability, reduced tendency to aggregate, improved expression and/or improved production yields relative to conventional fusion proteins. Novel fusion proteins that comprise at least one natural junction, compositions comprising the fusion proteins and methods of using the proteins are also disclosed.

### RELATED APPLICATIONS

This application is a continuation-in-part of International Application No. PCT/GB2006/004559, which designated the United States and was filed on Dec. 5, 2006, and this application claims the benefit of U.S. Provisional Application No. 60/761,708, filed on Jan. 24, 2006. The entire teachings of the above applications are incorporated herein by reference.

### BACKGROUND OF THE INVENTION

Fusion proteins are a recognized class of potentially effective therapeutic and diagnostic agents. One benefit provided by fusion protein technology is the possibility of designing a fusion protein that has desired function, enhanced desirable properties and/or decreased undesirable properties.

Fusion proteins contain component polypeptides which are derived from different parental proteins, and bonded or fused to each other through a peptide bond. Each component polypeptide in a fusion protein contributes to the properties of the fusion protein, and it is desirable for the component polypeptide to be fused at positions that do not result in a reduction in the activity of the component polypeptides. Thus, conventional fusion proteins generally are fused at positions that correspond to domain boundaries, or the loops between domains, in the native parental proteins. For example, a conventional chimeric antibody light chain is a fusion protein that contains a non-human antibody light chain variable domain that is fused to a human light chain constant domain.

One aspect of conventional fusion proteins that can limit commercial applications is that the amino acid sequence and structure surrounding the fusion site does not match the corresponding amino acid sequence of either of the parental proteins. As a result, the fusion protein contains a “non-self” amino acid sequence that includes the amino acids adjacent to the fusion site. Even when a fusion protein contains polypeptides derived from proteins from the same species (e.g., two human polypeptides are fused), the amino acid sequence at the fusion site will commonly comprise a non-self sequence generated by the juxtaposition of amino acid residues from different parental proteins. These non-self sequences can function as antibody and/or T-cell epitopes and render the fusion protein immunogenic, and can limit in vivo uses of the fusion protein, or render the fusion protein unsuitable for in vivo applications.

The juxtaposition of amino acid residues at the fusion site in conventional fusion proteins can also have other undesirable effects. For example, the juxtaposed amino acids can result in disruption of structural features important for expression, activity and/or stability. Consequently, conventional fusion proteins frequently form aggregates or oligomers, have low solubility and/or are more susceptible to proteolysis than are the parental proteins. In addition, conventional fusion proteins frequently can only be produced in lower yields than the parental proteins.

There is a need for improved fusion proteins and improved methods for designing and making fusion proteins.

### SUMMARY OF THE INVENTION

The invention relates to recombinant fusion proteins that contain natural junctions. The fusion proteins of the invention comprise at least two portions derived from two different polypeptides, and at least one natural junction between the two portions.

The recombinant fusion proteins can comprise a hybrid domain, that contains a first portion derived from a first polypeptide and a second portion derived from a second polypeptide, wherein the first polypeptide comprises a domain that has the formula (X1-Y-X2), and the second polypeptide comprising a domain that has the formula (Z1-Y-Z2), wherein Y is a conserved amino acid motif, X1 and Z1 are the amino acid motifs that are located adjacent to the amino-terminus of Y in said first polypeptide and said second polypeptide, respectively, and X2 and Z2 are the amino acid motifs that are located adjacent to the carboxy-terminus of Y in said first polypeptide and said second polypeptide, respectively, provided that if the amino acid sequences of X1 and Z1 are the same, the amino acid sequences of X2 and Z2 are not the same; and when the amino acid sequences of X2 and Z2 are the same, the amino acid sequences of X1 and Z1 are not the same.

If desired, the hybrid domain can be bonded to an amino-terminal amino acid sequence D, and/or bonded to a carboxy-terminal amino acid sequence E, such that the recombinant fusion protein comprises a structure that has the formula D-(X1-Y-Z2)-E, wherein D is absent or is an amino acid sequence that is adjacent to the amino-terminus of (X1-Y-X2) in said first polypeptide; and E is absent or is an amino acid sequence that adjacent to the carboxy-terminus of (Z1-Y-Z2) in said second polypeptide. In particular embodiments, D is present, E is present, or D and E are present.

In some embodiments, the hybrid domain (X1-Y-Z2) is a hybrid immunoglobulin variable domain, such as hybrid antibody variable domain. Y can be in framework region (FR) 4, for example, Y can be GlyXaaGlyThr (SEQ ID NO:386) or GlyXaaGlyThrXaa(Val/Leu) (SEQ ID NO:387). In such embodiments, X1 can be a portion of an antibody variable domain comprising FR1, complementarity determining region (CDR) 1, FR2, CDR2, FR3, and CDR3.

In other embodiments Y is in FR3, for example Y can be GluAspThrAla (SEQ ID NO:388), ValTyrTyrCys (SEQ ID NO:389), or GluAspThrAlaValTyrTyrCys (SEQ ID NO:390). In such embodiments, X1 can be a portion of an antibody variable domain comprising FR1, CDR1, FR2, and CDR2.

In some embodiments, the hybrid domain (X1-Y-Z2) is a hybrid immunoglobulin constant domain, such as a hybrid antibody constant domain. Y can be (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393) or ValThrVal (SEQ ID NO:394). For example, in particular embodiments, Y is selected from the group consisting of SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), and or ValThrVal (SEQ ID NO:394).

In some embodiments, D is absent, (X1-Y-Z2) is a hybrid immunoglobulin variable domain, and E is an immunoglobulin constant domain. The fusion protein can further comprise a second immunoglobulin variable domain that is amino terminal to or carboxyl terminal to (X1-Y-Z2).

In some embodiments, D is an immunoglobulin variable domain, and (X1-Y-Z2) is a hybrid immunoglobulin constant domain. In other embodiments, (X1-Y-Z2) is a hybrid immunoglobulin constant domain, and E is an immunoglobulin constant domain. In other embodiments, E is absent, (X1-Y-Z2) is a hybrid immunoglobulin constant domain, and the fusion protein comprises a further domain that is amino terminal to (X1-Y-Z2).

In other embodiments, D is an immunoglobulin constant domain, and (X1-Y-Z2) is a hybrid immunoglobulin constant domain.

The fusion protein of the invention, can comprise a first portion from a first polypeptide and a second portion from a second polypeptide wherein both polypeptides are members of the same protein superfamily. For example, the polypeptides can both be members of a protein superfamily is selected from the group consisting of the immunoglobulin superfamily, the TNF superfamily and the TNF receptor superfamily. Additionally or alternatively, the first polypeptide and said second polypeptide are both human polypeptides.

Generally X1, X2, Z1 and Z2 each, independently, consists of about 1 to about 200 amino acids. In some embodiments, the hybrid domain is about the size of an immunoglobulin variable domain or an immunoglobulin constant domain.

In more particular embodiments, the recombinant fusion protein comprises a hybrid immunoglobulin variable domain that is fused to an immunoglobulin constant domain. The hybrid immunoglobulin variable domain comprises a hybrid framework region (FR) that comprises a portion from a first immunoglobulin FR from a first immunoglobulin and a portion from a second immunoglobulin FR from a second immunoglobulin, the first immunoglobulin FR and the second immunoglobulin FR each comprise a conserved amino acid motif Y, and the hybrid immunoglobulin FR has the formula

(F¹-Y-F²)

wherein Y is the conserved amino acid motif;

F¹ is the amino acid motif located adjacent to the amino-terminus of Y in the first immunoglobulin FR; and

F² is the amino acid motif located adjacent to the carboxy-terminus of Y in the second immunoglobulin FR.

Y can located in FR1, FR2, FR3 or FR4 of the first immunoglobulin and of the second immunoglobulin.

In some embodiments, Y is located in FR4, and F² is the amino acid sequence that is adjacent to (peptide bonded to) the amino-terminus of an immunoglobulin constant domain in a naturally occurring protein comprising said immunoglobulin constant domain. In some embodiments, the immunoglobulin constant domain is an antibody light chain constant domain and said second immunoglobulin FR is a FR4 from an antibody light chain variable domain. For example, the antibody constant domain is a Cκ or Cλ, and said second antibody FR4 is a Vκ FR4 or Vλ FR4, respectively.

In some embodiments, the first immunoglobulin is a non-human immunoglobulin, such as an immunoglobulin from a mouse, rat, shark, fish, possum, sheep, pig, Camelid, rabbit or non-human primate. In such embodiments, the second immunoglobulin can be a human immunoglobulin. Preferably, in such embodiments, the hybrid FR is bonded to a human immunoglobulin constant domain.

In particular embodiments, the hybrid immunoglobulin variable domain is a hybrid antibody variable domain, and Y is GlyXaaGlyThr (SEQ ID NO:386). In such embodiments, F¹ can be Phe and F² is (Leu/Met/Thr)ValThrValSerSer (SEQ ID NO:420). Preferably, the fusion protein of this embodiment comprises a human antibody constant domain, such as an IgG CH1 domain.

In particular embodiments, the hybrid immunoglobulin variable domain is a hybrid antibody variable domain, Y is GlyXaaGlyThu (SEQ ID NO:386), F¹ is Trp and F² is (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425). Preferably, the fusion protein of this embodiment comprises a human antibody light chain constant domain.

In particular embodiments, the hybrid immunoglobulin variable domain is a hybrid antibody variable domain, and Y is GlyXaaGlyThrXaa(Val/Leu) (SEQ ID NO:387). In such embodiments, F¹ can be Phe, and F² can be ThrValSerSer (SEQ ID NO:419). Preferably, the fusion protein of this embodiment comprises a human antibody heavy chain constant domain, such as an IgG1 or IgG4 CH1 domain or IgG1 or IgG4 CH2 domain.

In particular embodiments, the hybrid immunoglobulin variable domain is a hybrid antibody variable domain. Y is GlyXaaGlyThrXaa(Val/Leu) (SEQ ID NO:387), F¹ is Trp, and F² is (Glu/Asp)IleLys (SEQ ID NO:458) or (Thr/Ile)(Val/Ile)Leu (SEQ ID NO:459). Preferably, the fusion protein of this embodiment comprises a human antibody light chain constant domain.

If desired, the recombinant fusion protein can comprises a structure that has the formula (F¹-Y-F²)-C_{L}, (F¹-Y-F²)-CH1, (F¹-Y-F²)-CH2, or (F¹-Y-F²)-Fc. The recombinant fusion protein can further comprises a second immunoglobulin variable domain, that is amino terminal or carboxy-terminal to (F¹-Y-F²).

The invention also relates to improved fusion proteins that comprise a non-human antibody variable region fused to a human antibody constant domain, the improvement comprising a hybrid FR4 in the non-human variable region that has the formula

(F¹-Y-F²)

wherein F¹ is Phe or Trp;

Y is GlyXaaGlyThr (SEQ ID NO:386), and F² is (Leu/Met/Thr)ValThrValSerSer (420), (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425); or

Y is GlyXaaGlyThrXaa(Val/Leu) (SEQ ID NO:387), and F² is ThrValSerSer (SEQ ID NO:419), (Glu/Asp)IleLys (SEQ ID NO:458) or (Thr/Ile)(Val/Ile)Leu (SEQ ID NO:459).

The recombinant fusion protein can comprise an immunoglobulin variable domain fused to a hybrid immunoglobulin constant domain, The hybrid immunoglobulin constant domain comprises a portion from a first immunoglobulin constant domain and a portion from a second immunoglobulin constant domain, the first immunoglobulin constant domain and the second immunoglobulin constant domain each comprising a conserved amino acid motif Y. The hybrid immunoglobulin constant domain has the formula

C¹-Y-C²

wherein Y is said conserved amino acid motif;

C¹ is the amino acid motif adjacent to the amino-terminus of Y in the first immunoglobulin constant region;

C² is the amino acid motif adjacent to the carboxy-terminus of Y in the second immunoglobulin constant region.

In some embodiments, the hybrid immunoglobulin constant domain is a hybrid antibody constant domain comprising a portion from a first antibody constant domain and a portion from a second antibody constant domain. the hybrid antibody constant domain can be a hybrid antibody CH1, a hybrid antibody hinge, a hybrid antibody CH2, or a hybrid antibody CH3.

In some embodiments, first antibody constant domain and said second antibody constant domain are from different species.

In other embodiments, the second antibody constant domain is a human antibody constant domain. Alternatively or additionally, first antibody constant domain is a mouse, rat, shark, fish, possum, sheep, pig, Camelid, rabbit or non-human primate constant domain.

In some embodiments, the fusion protein comprises an immunoglobulin variable domain that is a non-human antibody variable domain and the first constant domain is the corresponding non-human CH1 domain, Cλ domain or Cκ domain. In some embodiments, the first antibody constant domain is a light chain constant domain, and said second antibody constant domain is a heavy chain constant domain.

In other embodiments, the first antibody constant domain is a Camelid heavy chain constant domain, and said second antibody constant domain is a heavy chain constant domain. If desired, a VHH can be amino terminal to the hybrid constant domain.

In some embodiments, first antibody constant domain and said second antibody constant domain are of different isotypes. Preferably, the second antibody constant domain is an IgG constant domain.

In some embodiments, the fusion protein comprise an antibody variable domain that is a light chain variable domain and the first antibody constant domain is a light chain constant domain. In such embodiments, the second antibody constant domain can be a human antibody heavy chain constant domain or a human antibody light chain constant domain. In some embodiments, the human antibody heavy chain constant domain is a CH1, a hinge, a CH2, or a CH3.

In particular embodiments, Y is (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393), or ValThrVal (SEQ ID NO:394). In some of these embodiments, the second antibody constant domain is a human antibody constant domain, such as Cκ, Cλ, a CH1, a hinge, a CH2 and a CH3.

In particular embodiments, the recombinant fusion protein comprises a human light chain variable domain that is fused to a hybrid human CH1 domain, and

C¹ is GlnProLysAla (SEQ ID NO:466) or ThrValAla (SEQ ID NO:467),

Y is (Ala/Gly)ProSerVal (SEQ ID NO:468), and

C² is the amino acid motif adjacent to the carboxy-terminus of Y in human IgG CH1.

In particular embodiments, the recombinant fusion protein comprises a human light chain variable domain that is fused to a hybrid human CH2, wherein:

C¹ is GinProLysAla (SEQ ID NO:466) or ThrValAla (SEQ ID NO:467),

Y is (Ala/Gly)ProSerVal (SEQ ID NO:468), and

C² is the amino acid motif adjacent to the carboxy-terminus of Y in human IgG CH2.

In particular embodiments, the recombinant fusion protein comprises a human heavy chain variable domain that is fused to a hybrid human CH2, wherein

C¹ is SerThrLys (SEQ ID NO:469),

Y is (Ala/Gly)ProSerValPhe (SEQ ID NO:470), and

C² is the amino acid motif adjacent to the carboxy-terminus of Y in human IgG CH2.

In particular embodiments, the recombinant fusion protein comprises a human lambda chain variable domain that is fused to a hybrid human Cκ, and wherein

C¹ is GlnProLysAla (SEQ ID NO:466),

Y is (Ala/Gly)ProSerVal (SEQ ID NO:468), and

C² is the amino acid motif adjacent to the carboxy-terminus of Y in human Cκ.

In particular embodiments, the recombinant fusion protein comprises a human heavy chain variable domain that is fused to a hybrid human Cκ, wherein

C¹ is SerThrLys (SEQ ID NO:469),

Y is (Ala/Gly)ProSerValPhe (SEQ ID NO:470), and

C² is the amino acid motif adjacent to the carboxy-terminus of Y in human Cκ.

In particular embodiments, the recombinant fusion protein comprises a human kappa chain variable domain that is fused to a hybrid human Cλ, and wherein

C¹ is ThrValAla (SEQ ID NO:467),

Y is (Ala/Gly)ProSerVal (SEQ ID NO:468), and

C² is the amino acid motif adjacent to the carboxy-terminus of Y in human Cλ.

In particular embodiments, the recombinant fusion protein comprises a human heavy chain variable domain that is fused to a hybrid human Cλ, wherein

C¹ is SerThrLys (SEQ ID NO:469),

Y is (Ala/Gly)ProSerVal (SEQ ID NO:468), and

C² is the amino acid motif adjacent to the carboxy-terminus of Y in human Cλ.

The invention also relates to a recombinant fusion protein comprising a first portion derived from a first polypeptide and a second portion derived from a second polypeptide, wherein said first polypeptide comprises a structure having the formula (A)-L1, wherein (A) is an amino acid sequence present is said first polypeptide; and L1 is an amino acid motif comprising 1 to about 50 amino acids that are adjacent to the carboxy-terminus of (A) in said first polypeptide; wherein said fusion polypeptide has the formula

(A)-L1-(B);

wherein (B) is said portion derived from said second polypeptide; with the proviso that at least one of (A) and (B) is a domain, and when (A) and (B) are both antibody variable domains

a) (A) and (B) are each human antibody variable domains;

b) (A) and (B) are each antibody heavy chain variable domains;

c) (A) and (B) are each antibody light chain variable domains;

d) (A) is an antibody light chain variable domain and (B) is an antibody heavy chain variable domain; or

e) (A) is a VHH and (B) is an antibody light chain variable domain; or with the proviso that when (A) and (B) are both antibody variable domains the following is excluded from the invention, (A)-L1-(B) where (A) is a mouse VH, (B) is a mouse VL and L1 is SerAlaLysThrThrPro (SEQ ID NO:537), SerAlaLysThrThrProLysLeuGlyGly (SEQ ID NO:538), AlaLysThrThrProLysLeuGluGluGlyGluPheSerGluAlaArgVal (SEQ ID NO:539), or AlaLysThrThrProLysLeuGluGlu (SEQ ID NO:540).

In some embodiments, the first polypeptide is an antibody variable domain. The second polypeptide can be an immunoglobulin constant region. In some embodiments, (B) comprises at least a portion of an antibody CH1, at least a portion of an antibody hinge, at least a portion of an antibody CH2, or at least a portion of an antibody CH3.

In some embodiments, (A) is an antibody light chain variable domain. In such embodiments, L1 comprises one to about 50 contiguous amino-terminal amino acids of Cκ or Cλ. In other embodiments, (A) is an antibody heavy chain variable domain, such as a VH or a VHH. In such embodiments, L1 can comprise one to about 50 contiguous amino-terminal amino acids of CH1.

In some embodiments, (A) is an antibody heavy chain variable domain and (B) is an antibody heavy chain variable domain, or (A) is an antibody light chain variable domain and (B) is an antibody heavy chain variable domain or an antibody light chain variable domain. For example, in certain embodiments (A) is a Vκ and (B) is a Vκ; (A) is a Vκ and (B) is a Vλ; (A) is a Vκ and (B) is a VH or a VHH; (A) is a Vλ and (B) is a Vκ; (A) is a Vλ and (B) is a Vλ; or(A) is a Vλ and (B) is a VH or a VHH.

In some embodiments (A) is a VH and L1 comprises the first 3 to about 12 amino acids of CH1; (A) is a Vκ and L1 comprises the first 3 to about 12 amino acids of Cκ; or (A) is a Vλ and L1 comprises the first 3 to about 12 amino acids of Cλ.

In certain embodiments (A) is an antibody variable domain comprising FR1, CDR1, FR2, CDR3, FR3 and CDR3 of a antibody light chain variable domain and FR4 comprising the amino acid sequence GlyGlnGlyThrLysValThrValSerSer (SEQ ID NO:472); and L1 comprises the first 3 to about 12 amino acids of CH1. In these embodiments, L1 can be AlaSerThr (473), AlaSerThrLysGlyProSer (SEQ ID NO:474), or AlaSerThrLysGlyProSerGly (SEQ ID NO:475).

In certain embodiments, (A) is an antibody variable domain comprising FR1, CDR1, FR2, CDR3, FR3 and CDR3 of a VH or Vκ domain and FR4 comprising the amino acid sequence GlyXaaGlyThr(Lys/Gln/Glu)(Val/Leu)(Thr/Ile)ValLeu (SEQ ID NO:476); and L1 comprises the first 3 to about 12 amino acids of Cλ. In other embodiments, (A) is an antibody variable domain comprising FR1, CDR1, FR2, CDR3, FR3 and CDR3 of a VH or Vλ domain and FR4 comprising the amino acid sequence GlyGlnGlyThrLysValGluIleLysArg (SEQ ID NO:477); and L1 comprises the first 3 to about 12 amino acids of Cκ.

In other embodiments, (A) is an immunoglobulin constant domain, such as an antibody constant domain. In other embodiments, (A) is a nonhuman immunoglobulin constant domain, and (B) is derived from a human polypeptide.

In some embodiments, the second polypeptide is selected from the group consisting of a cytokine, a cytokine receptor, a growth factor, a growth factor receptor, a hormone, a hormone receptor, an adhesion molecule, a haemostatic factor, a T cell receptor, a T cell receptor chain, a T cell receptor variable domain, enzyme, polypeptide comprising or consisting of an antibody variable domain, or a functional portion of any one of the foregoing.

In other embodiments, the first polypeptide is selected from the group consisting of a cytokine, a cytokine receptor, a growth factor, a growth factor receptor, a hormone, a hormone receptor, an adhesion molecule, a haemostatic factor, a T cell receptor, a T cell receptor chain, a T cell receptor variable domain, enzyme, polypeptide comprising or consisting of an antibody variable domain, or a functional portion of any one of the foregoing. In such embodiments, the second polypeptide can be an immunoglobulin constant region or Fc portion of an immunoglobulin constant region.

The invention relates to a recombinant fusion protein comprising a first portion that is an immunoglobulin variable domain and a second portion, wherein said first portion is bonded to said second portion through a linker, and the recombinant fusion protein has the formula

(A′)-L2-(B)

wherein (A′) is said immunoglobulin variable domain and comprises framework (FR) 4, L2 is said linker, wherein L2 comprises one to about 50 contiguous amino acids that are adjacent to the carboxy-terminus of said FR4 in a naturally occurring immunoglobulin that comprises said FR4; and (B) is said second portion;

with the proviso that L2-(B) is not a C_{L} or CH1 domain that is peptide bonded to said FR4 in a naturally occurring antibody that comprises said FR4, and when (A) and (B) are both antibody variable domains

a) (A) and (B) are each human antibody variable domains;

b) (A) and (B) are each antibody heavy chain variable domains;

c) (A) and (B) are each antibody light chain variable domains;

d) (A) is an antibody light chain variable domain and (B) is an antibody heavy chain variable domain; or

e) (A) is a VHH and (B) is an antibody light chain variable domain; or with the proviso that when (A) and (B) are both antibody variable domains the following is excluded from the invention, (A)-L1-(B) where (A) is a mouse VH, (B) is a mouse VL and L1 is SerAlaLysThrThrPro (SEQ ID NO:537), SerAlaLysThrThrProLysLeuGlyGly (SEQ ID NO:538), AlaLysThrThrProLysLeuGluGluGlyGluPheSerGluAlaArgVal (SEQ ID NO:539), or AlaLysThrThrProLysLeuGlyGly (SEQ ID NO:540).

In some embodiments (A′) is an antibody heavy chain variable domain or a hybrid antibody variable domain. In some embodiments antibody heavy chain variable domain or a hybrid antibody variable domain each comprise a FR4 that comprises the amino acid sequence GlyXaaGlyThr(Leu/Met/Thr)ValThrValSerSer (SEQ ID NO:478). In these embodiments, L2 can comprise one to about 50 contiguous amino acids from the amino-terminus of CH1. In particular embodiments L2 comprises AlaSerThr (SEQ ID NO:473), AlaSerThrLysGlyProSer (SEQ ID NO:474), or AlaSerThrLysGlyProSerGly (SEQ ID NO:475).

In some embodiments (A′) is a hybrid antibody variable domain or a Vκ that comprise a FR4 that comprises the amino acid sequence GlyXaaGlyThr(Lys/Arg)(Val/Leu)(Glu/Asp)IleLysArg (SEQ ID NO: 485). In these embodiments, L2 can comprises one to about 50 contiguous amino acids from the amino-terminus of Cκ. In particular embodiments L2 comprises ThrValAla (SEQ ID NO:467), ThrValAlaAlaProSer (SEQ ID NO:490), or ThrValAlaAlaProSerGly (SEQ ID NO:491). In some embodiments (A′) is a hybrid antibody variable domain or a Vλ that comprises a FR4 that comprises the amino acid sequence GlyXaaGlyThr(Lys/Gln/Glu)(Val/Leu)(Thr/Ile)(Val/Ile)Leu (SEQ ID NO:492).

In some embodiments, (B) comprises an antibody light chain variable domain or an antibody heavy chain variable domain. In other embodiments, (B) comprises at least a portion of an immunoglobulin constant region, for example at the amino-terminus of (B). The immunoglobulin constant region can be an IgG constant region, such as an IgG1 constant region or an IgG4 constant region. In some embodiments (B) comprises at least a portion of CH1, at least a portion of hinge, at least a portion of CH2 or at least a portion of CH3.

In particular embodiments, (B) comprises at least a portion of hinge that comprises ThrHisThrCysProProCysPro (SEQ ID NO:520). Additionally, (B) can further comprises CH2-CH3. In other embodiments, (B) comprises a portion of CH1-hinge-CH2-CH3, hinge-CH2-CH3, CH2-CH3, or CH3.

The invention relates to a recombinant fusion protein comprising a first portion and a second portion derived from an immunoglobulin constant region. The first portion is bonded to said second portion through a linker, and the recombinant fusion protein has the formula

(A)-L3-(C³)

wherein (A) is said first portion, (C³) is said second portion derived from an immunoglobulin constant region; and L3 is said linker, wherein L3 comprises one to about 50 contiguous amino acids that are adjacent to the amino-terminus of (C³) in a naturally occurring immunoglobulin that comprises (C³), with the proviso that (A) is not an antibody variable domain found in said naturally occurring immunoglobulin.

In some embodiments, (C³) comprises at least on antibody constant domain, such as a human antibody constant domain. In some embodiments the antibody constant domain is an IgG constant domain, such as an IgG1 constant domain or an IgG4 constant domain.

In some embodiments, (C³) comprises CH3. In these embodiments, L3 comprises one to about 50 contiguous amino acids from the carboxy-terminus of CH2. In other embodiments, (C³) comprises CH2 or CH2-CH3. In these embodiments, L3 comprises one to about 34 contiguous amino acids from the carboxy-terminus of hinge. For example, L3 can comprise ThrHisThrCysProProCysPro (SEQ ID NO:520) or GlyThrHisThrCysProProCysPro (SEQ ID NO:521).

In some embodiments, (C³) comprises hinge. In these embodiments, L3 comprises one to about 50 contiguous amino acids from the carboxy-terminus of CH1. In some embodiments, (C³) comprises CH1. In these embodiments, L3 comprises one to about 50 contiguous amino acids from the carboxy-terminus of an antibody heavy chain V domain. For example, L3 comprises GlyXaaGlyThr(Leu/Met/Thr)ValThrValSerSer (SEQ ID NO:478).

In some embodiments, the antibody constant domain is a Cκ or a Cλ. In such embodiments, L3 comprises one to about 50 contiguous amino acids from the carboxy-terminus of an antibody light chain V domain. For example, when the antibody constant domain is a Cκ, L3 can comprises GlyXaaGlyThr(Lys/Arg)(Val/Leu)(Glu/Asp)IleLysArg (SEQ ID NO:485). When the antibody constant domain is a Cλ, L3 can comprises GlyXaaGlyThr(Lys/Gln/Glu)(Val/Leu)(Thr/Ile)(Val/Ile)Leu (SEQ ID NO:492).

In certain embodiments, (A) is selected from the group consisting of a cytokine, a cytokine receptor, a growth factor, a growth factor receptor, a hormone, a hormone receptor, an adhesion molecule, a haemostatic factor, a T cell receptor, a T cell receptor chain, a T cell receptor variable domain, enzyme, polypeptide comprising or consisting of an antibody variable domain, or a functional portion of any one of the foregoing.

The invention also relates to a recombinant fusion protein comprising a first portion derived from an antibody variable domain and a second portion derived from a second polypeptide, wherein said antibody variable domain comprises a structure having the formula (A)-L1, wherein (A) consists of CDR3; L1 consists of FR4, wherein said fusion polypeptide has the formula (A)-L1-(B), wherein (B) is said portion derived from said second polypeptide.

In some embodiments, the second polypeptide is an immunoglobulin constant region. In other embodiments, (B) comprises at least a portion of an antibody CH1, at least a portion of an antibody hinge, at least a portion of an antibody CH2, or at least a portion of an antibody CH3.

The invention also relates to an isolated recombinant nucleic acid molecule encoding a recombinant fusion protein comprising a natural junction as described herein, and to a host cell comprising a recombinant nucleic acid molecule encoding a recombinant fusion protein comprising a natural junction as described herein

The invention also relates to a method of producing a recombinant fusion protein comprising maintaining a host cell of the invention under conditions suitable for expression of a recombinant nucleic acid encoding the fusion protein comprising a natural junction, whereby said recombinant nucleic acid is expressed and said recombinant fusion protein is produced. In certain embodiments, the method further comprises isolating said recombinant fusion protein.

The invention also relates to recombinant fusion protein comprising a natural junction as described herein for use in therapy, diagnosis and/or prophylaxis. The invention also relates to the use of a recombinant fusion protein comprising a natural junction as described herein for the manufacture of a medicament for therapy, diagnosis and/or prophylaxis in a human, with reduced likelihood of inducing an immune response.

The invention also relates to a method of therapy, diagnosis and/or prophylaxis in a human comprising administering to said human an effective amount of a recombinant fusion protein comprising a natural junction as described herein, whereby the likelihood of inducing an immune response is reduced in comparison to a corresponding fusion protein that does not contain a natural junction.

The invention also relates to use of a natural junction for preparing a recombinant fusion protein for human therapy, diagnosis and/or prophylaxis, with reduced likelihood of inducing an immune response in comparison to a corresponding fusion protein that does not contain a natural junction.

The invention relates to use of a natural junction for preparing a recombinant fusion protein for human therapy, diagnosis and/or prophylaxis, with reduced propensity to aggregate in comparison to a corresponding fusion protein that does not contain a natural junction.

The invention relates to use of a natural junction for preparing a recombinant fusion protein for human therapy, diagnosis and/or prophylaxis, wherein said recombinant fusion protein is expressed at higher levels in comparison to a corresponding fusion protein that does not contain a natural junction.

The invention relates to use of a natural junction for preparing a recombinant fusion protein for human therapy, diagnosis and/or prophylaxis, wherein said recombinant fusion protein has enhanced stability in comparison to relative to a corresponding fusion protein that does not contain a natural junction.

The invention relates to use of a natural junction for preparing a recombinant fusion protein comprising a first portion (A) and a second portion (B), and at least one natural junction between (A) and (B), and wherein said recombinant fusion protein has reduced propensity to aggregate in comparison to a corresponding fusion protein comprising (A) and (B), wherein the interface of (A) and (B) is not a natural junction.

The invention relates to use of a natural junction for preparing a recombinant fusion protein comprising a first portion (A), a second portion (B), and at least one natural junction between (A) and (B), wherein said recombinant fusion protein is expressed at higher levels in comparison to a corresponding fusion protein comprising (A) and (B), wherein said corresponding fusion protein does not contain a natural junction between (A) and (B).

The invention relates to use of a natural junction for preparing a recombinant fusion protein comprising a first portion (A), a second portion (B), and at least one natural junction between (A) and (B), wherein said recombinant fusion protein has enhanced stability in comparison to a corresponding fusion protein comprising (A) and (B), wherein said corresponding fusion protein does not contain a natural junction between (A) and (B).

The invention relates to a pharmaceutical composition comprising a recombinant fusion protein comprising a natural junction as described herein and a physiologically acceptable carrier.

The invention relates to a method of designing or producing a fusion protein comprising a first portion and a second portion that are fused at a natural junction, wherein said first portion is derived from a first polypeptide and said second portion is derived from a second polypeptide. The method comprises analyzing the amino acid sequence of said first polypeptide or a portion thereof and the amino acid sequence of said second polypeptide or a portion thereof to identify a conserved amino acid motif present in both of the analyzed sequences; and preparing a fusion protein which has the formula

A-Y-B;

wherein A is said first portion, Y is said conserved amino acid motif, B is said second portion, and wherein said first polypeptide comprises A-Y, and said second polypeptide comprises Y-B.

In some embodiments, the second polypeptide comprises an immunoglobulin constant domain, such as a human immunoglobulin constant domain or a nonhuman immunoglobulin constant domain. In particular embodiments, the second polypeptide comprises an antibody constant domain.

In some embodiments, the second polypeptide and B comprise an antibody heavy chain constant domain, such as a hinge region, a portion of CH1-hinge-CH2-CH3, hinge-CH2-CH3, CH2-CH3, or CH3. Preferably, the constant domain is a human antibody heavy chain constant domain, such as an IgG (e.g., IgG1 constant domain or an IgG4 constant domain).

In some embodiments, the first polypeptide is selected from the group consisting of a cytokine, a cytokine receptor, a growth factor, a growth factor receptor, a hormone, a hormone receptor, an adhesion molecule, a haemostatic factor, a T cell receptor, a T cell receptor chain, a T cell receptor variable domain, enzyme, polypeptide comprising or consisting of an antibody variable domain, or a functional portion of any one of the foregoing

In some embodiments, the first polypeptide and A comprise an immunoglobulin variable domain, such as a human immunoglobulin variable domain or a nonhuman immunoglobulin variable domain. In certain embodiments, the first polypeptide comprises non-human antibody variable domain or a human antibody variable domain. In these embodiments, the second polypeptide can be selected from the group consisting of a cytokine, a cytokine receptor, a growth factor, a growth factor receptor, a hormone, a hormone receptor, an adhesion molecule, a haemostatic factor, a T cell receptor, a T cell receptor chain, a T cell receptor variable domain, enzyme, polypeptide comprising or consisting of an antibody variable domain, or a functional portion of any one of the foregoing.

In some embodiments, the first polypeptide is a first antibody chain, the second polypeptide is a second antibody chain. In these embodiments, Y is in the variable domain of said first antibody chain and the variable domain of said second antibody chain. In one embodiment, Y is in framework region (FR) 4. In these embodiments, Y can be GlyXaaGlyThr (SEQ ID NO:386) or GlyXaaGlyThrXaa(Val/Leu) (SEQ ID NO:387). In other embodiments, Y is in FR3. In these embodiments, Y can be GluAspThrAla (SEQ ID NO:388), ValTyrTyrCys (SEQ ID NO:389), or GluAspThrAlaValTyrTyrCys (SEQ ID NO:390). In other embodiments, Y is in a constant domain of said first antibody chain and a constant domain of said second antibody chain. In these embodiments, Y can be (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393) or ValThrVal (SEQ ID NO:394).

In some embodiments, the first antibody chain, and said second antibody chain are from different species. In other embodiments, the first antibody chain, and said second antibody chain are from the same species. In particular embodiments, the first antibody chain and said second antibody chain are human.

In some embodiment the fusion protein further comprises a third portion located amino terminally to A. In some embodiment, the third portion comprises an immunoglobulin variable domain.

In some embodiments, the first polypeptide and said second polypeptide are both members of the same protein superfamily. For example, the first polypeptide and the second polypeptide can be member of a protein superfamily selected from the group consisting of the immunoglobulin superfamily, the TNF superfamily and the TNF receptor superfamily.

### BRIEF DESCRIPTION OF THE DRAWINGS

FIG. 1A illustrates the structure of a typical human Fab′ fragment.

FIG. 1B illustrates a cluster of five residues in a typical human Fab′ fragment (three highly conserved residues in VH (H11 [Leu or Val], H110 [Thr] and H112 [Ser]) and two highly conserved residues in CH1 (H148 [Phe] and H149 [Pro]). This cluster provides a degree of controlled flexibility that changes the orientation of Vκ-VH domains relative to Cκ-CH1 domains in immunoglobulins.

FIG. 1C illustrates the typical interactions found between Vκ and Cκ domains of a typical human Fab′ fragment.

FIGS. 2A and 2B are alignments of the amino acid sequences in human antibody and TCR J-segments illustrating conserved motifs. The aligned amino acid sequences are from human IgH J-segments (SEQ ID NOS:1-6), human Igκ J-segments (SEQ ID NOS:7-11), human Igλ J-segments (SEQ ID NOS:12-18), human TCRβ J-segments (SEQ ID NOS:19-32), human TCRγ J-segments (SEQ ID NOS:33-37), human TCRδ J-segments (SEQ ID NOS:38-41) and human TCRα segments (SEQ ID NOS:42-98).

FIG. 3 illustrates a conserved motif in antibody heavy chain (IgH) J-segments from various species. Amino acid alignments of Mouse IgH J-segments (SEQ ID NOS:99-102), Llama IgH J-segments (SEQ ID NO:103-107), Sheep IgH J-segments (SEQ ID NOS:108-113) and a Pig IgH J-segment (SEQ ID NO:114) are shown.

FIG. 4 illustrates a conserved motif in antibody 1κ chain (Igκ) J-segments from various species and a conserved motif in antibody λ chain (Igλ) J-segments from various species. Amino acid sequence alignments of Mouse Igκ J-segments (SEQ ID NOS:115-119) and Igλ J-segments (SEQ ID NOS:126-130), Possum Igκ J-segments (SEQ ID NOS:120-121) and Igλ J-segments (SEQ ID NOS:131-133), and Sheep Igκ J-segments (SEQ ID NOS:122-125) and Igλ J-segment (SEQ ID NO:134) are shown.

FIG. 5 illustrates the conserved motifs in mouse antibody constant domains. The amino acid sequence alignments show conserved motifs in CH1 (SEQ ID NOS:135-143), CH2 (SEQ ID NOS:144-151), CH3 (SEQ ID NOS:152-160), Hinge (SEQ ID NOS:161-171), Cκ (SEQ ID NOS:172-173), and Cλ regions (SEQ ID NOS:174-176) of mouse Ig.

FIG. 6 illustrates the conserved motifs in human antibody constant domains. The amino acid sequence alignments show a conserved motifs in CH1 (SEQ ID NOS:177-185), CH2 (SEQ ID NOS:186-194), CH3 (SEQ ID NOS:195-203), Hinge (SEQ ID NOS:204-210), Cκ (SEQ ID NO:211), and Cλ regions (SEQ ID NOS:212-216) of human Ig.

FIG. 7 illustrates the conserved motifs in camel antibody constant domains and human TCR constant domains. Amino acid sequence alignments show the conserved motifs in CH1 (SEQ ID NO:217), CH2 (SEQ ID NOS:218-219), CH3 (SEQ ID NOS:220-221) and Hinge (SEQ ID NOS:222-223) regions of camel antibody. An alignment of several human TCR constant domains is also shown (SEQ ID NOS:224-230).

FIG. 8 illustrates the conserved motifs in nurse shark heavy chain (IgH) J-segments (SEQ ID NOS:231-282) and nurse shark IgI J-segments (SEQ ID NOS:283-288).

FIGS. 9A and 9B illustrate a conserved motif in mouse TCR J-segments. Amino acid sequence alignments of mouse TCRα J-segments (SEQ ID NOS:289-338), mouse TCRβ J-segments (SEQ ID NOS:339-351) and mouse TCRδ J-segments (SEQ ID NOS:352-353) are shown.

FIGS. 10A and 10B are alignments of the amino acid sequences of several Camelid VHHs (SEQ ID NOS:354-383), and show conserved motifs present in the VHHs (marked with*).

FIG. 11 is an alignment of the germline amino acid sequence of human DP-47 variable domain (SEQ ID NO:384), and the amino acid sequence of Camelid VHH#12B variable domain (SEQ ID NO:385). The alignments reveal that there are 4 amino acid differences in FR1 (positions 1, 5, 28 and 30), 5 amino acid differences in FR3 (positions 74, 76, 83, 84 and 93), and that there are amino acid motifs that are conserved in the sequences.

### DETAILED DESCRIPTION OF THE INVENTION

Within this specification embodiments have been described in a way which enables a clear and concise specification to be written, but it is intended and will be appreciated that embodiments may be variously combined or separated without parting from the invention. To enable the invention to be described clearly and concisely, this specification contains formulae that represent partial structures of the disclosed fusion proteins. These formulae depict portions of the fusion protein that are located amino terminally to carboxy terminally (from left to right in the formulae) as is conventional in the art.

Within this specification, the term “about” is preferably interpreted to mean optionally plus or minus 50%, more preferably optionally plus or minus 20%, even more preferably optionally plus or minus 10%, even more preferably optionally plus or minus 5%, even more preferably optionally plus or minus 2%, even more preferably optionally plus or minus 1%.

“Fusion protein” is a term of art that refers to a continuous polypeptide chain that contains parts or portions that are derived from different parental amino acid sequences (e.g., proteins). The portions of a fusion protein can be directly bonded to each other or indirectly bonded through, for example, a peptide linker. A fusion protein can contain two or more portions that are derived from two or more different polypeptides.

As used herein “junction” refers to the site at which two amino acid sequences that are derived from two different polypeptides are joined in a fusion protein.

As used herein a “natural junction” refers to a junction in a fusion protein that has an amino acid sequence that is the same as the amino acid sequence found at the corresponding position of one or both of the parental polypeptides. For example, as illustrated herein in Scheme 1 using hypothetical parental proteins X and Y, a fusion protein can be prepared that contains the conceptual amino acid sequence XXXXXX11111111111YYYYY, in which XXXXXX are amino acids derived from parental protein X, YYYYYY are amino acids derived from parental protein Y, and 11111111111 is a conserved amino acid motif present in both parental proteins. The fusion protein contains a natural junction because the amino acid sequence XXXXX11111111111 is the same as the amino acid sequence at the corresponding location in parental protein X. In this example, the fusion protein contains two natural junctions because the amino acid sequence 11111111111YYYYYY is also the same as the amino acid sequence at the corresponding location in parental protein Y.

As used herein, “immunoglobulin variable domain” refers to antibody variable domains and TCR variable domains. An immunoglobulin variable domain can be derived from an antibody or TCR of desired origin (e.g., of human origin) or from a library prepared using antibody variable region genes or TCR variable region genes, such as human antibody variable region genes or human TCR variable region genes. See, e.g., Kabat, E. A. et al., Sequences of Proteins of Immunological Interest, Fifth Edition, U.S. Department of Health and Human Services, U.S. Government Printing Office (1991).

As used herein, “immunoglobulin constant domain” refers to antibody constant domains (e.g., CH1, hinge, CH2, CH3) and TCR constant domains. An immunoglobulin constant domain can be derived from an antibody or TCR of desired origin (e.g., of human origin) or by any suitable method using readily available antibody constant domain sequence information. See, e.g., Kabat, E. A. et al., Sequences of Proteins of Immunological Interest, Fifth Edition, U.S. Department of Health and Human Services, U.S. Government Printing Office (1991).

As used herein, “human” refers to Homo sapiens and to polypeptides, and portions of polypeptides, of human origin. Such polypeptides or portions thereof are substantially non-immunogenic in humans. Human polypeptides and portions of human polypeptides include polypeptides or portions that contain the same amino acid sequence as a polypeptide or portion thereof that occurs naturally in a human. Human polypeptides or portions thereof can be produced using any suitable method, and include polypeptides or portions thereof that are isolated from a human (e.g., of sample obtained from a human), and those that are produced recombinantly or synthetically.

As used herein, “human immunoglobulin variable domain,” “human antibody variable domain” (e.g., human V_{H}, human V_{L}, human V_{κ}, human V_{λ}, and the like), “human TCR variable domain” refer to variable domains in which one or more framework regions are encoded by a human germline immunoglobulin gene segment, or that have up to 5 amino acid differences relative to the amino acid sequence encoded by a human germline immunoglobulin gene segment. Immunoglobulin variable domains contain hypervariable regions (e.g., CDR1, CDR2, CDR3) which by their nature contain diverse amino acid sequences. In accordance with accepted standards in the immunoglobulin arts, the presence of amino acids in hypervariable regions that are not encoded by the human germline does not render an immunoglobulin variable domain non-human. Human immunoglobulin variable domains can contain one or more CDRs that are not encoded by the human germline, and can additionally contain up to 10 additional amino acids that are not in the CDRs and are not encoded by the human germline. Preferably, the amino acid sequences of FW1, FW2, FW3 and FW4 are each encoded by a human germline immunoglobulin gene segment, or collectively contain up to 10 amino acid differences relative to the amino acid sequences of the corresponding framework regions encoded by the human germline immunoglobulin gene segment.

As used herein “hybrid domain” refers to a recombinant domain that comprises a portion from a first domain of the same type and a portion from a second domain of the same type. For example, a hybrid antibody variable domain can comprise FR1-CDR1-FR2-CDR2-FR3-CDR3 and a portion of FR4 from a Vκ, and a portion of FR4 from an antibody heavy chain variable domain. Domains of the same type include immunoglobulin variable domains (e.g., antibody light and heavy chain variable domains, and TCR variable domains) and immunoglobulin constant domains (e.g., antibody light and heavy chain constant domains, TCR constant domains).

As used herein “conserved amino acid motif” refers to a region containing one to about 50 contiguous amino acids with conserved amino acid sequence that is present in one or more polypeptides, and in certain fusion proteins of the invention that contain portions derived from such polypeptides. The amino acid sequences of the conserved amino acid motif may or may not be identical in individual polypeptides that contain the conserved amino acid motif. As is known in the art, amino acid sequence motifs may differ in amino acid sequence to some degree, but the overall sequence diversity of an amino acid motif is limited by the presence of invariant amino acid residues, and of positions with limited variation, such as conservative amino acid substitutions. Conserved amino acid motifs, such as the GlyXaaGlyThr (SEQ ID NO:386) motif present in framework 4 of immunoglobulin variable domains from many species, can be identified in the conventional manner by alignment of amino acid sequences. Preferably, the amino acid sequences of the conserved amino acid motifs present in two or more polypeptides have at least about 50%, at least about 60%, at least about 70%, at least about 75%, at least about 80%, at least about 85%, at least about 90%, at least about 95%, at least about 96%, at least about 97%, at least about 98%, or at least about 99% amino acid sequence similarity or identity to each other over the length of the motif.

As used herein, a first amino acid, amino acid sequence or motif is “adjacent” to a second amino acid, amino acid sequence or motif when the first amino acid sequence or motif is peptide bonded directly to the second amino acid sequence or motif to create a continuous polypeptide chain.

Amino acid and nucleotide sequence alignments and homology, similarity or identity, as defined herein are preferably prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, Proc. Natl. Acad. Sci. USA 87(6):2264-8 (1990).

The invention relates to recombinant fusion proteins that contain natural junctions. The fusion proteins of the invention generally comprises a conserved amino acid sequence motif that is present in two polypeptides that are to be fused. The amino acid sequence that is adjacent to the amino-terminus of the conserved motif is the same as the amino sequence that is adjacent to the amino-terminus of the conserved motif in one of the original polypeptides, and the amino acid sequence that is adjacent to the carboxy-terminus of the conserved motif is the same as the amino acid sequence that is adjacent to the carboxy-terminus of the conserved motif in the other original polypeptide.

The fusion proteins of the invention provide several advantages over conventional fusion proteins. For example, domain interactions in proteins make important contributions to the stability (e.g., aggregation resistance, protease resistance) of proteins. However, domain interaction in fusion proteins are frequently altered because the components of conventional fusion proteins are typically fused at domain boundaries. The resulting juxtaposition of domains from different parental proteins can result in low stability.

One feature of fusion proteins that contain natural junctions is that they generally are designed to preserve domain interactions, thereby improving stability and reducing immunogenicity of the fusion protein. Preferably, in some embodiments, the potential for domain repulsion is reduced in the fusion proteins of the invention, which also reduces susceptibility to proteolysis. A related common problem with conventional fusion proteins is that during production, a fraction of the recombinant protein usually forms soluble or insoluble aggregates, lowering the yield of desired soluble monomeric fusion proteins. The improved stability of the fusion proteins of the invention can also or alternatively result in less aggregation, improved expression and/or improved production yields. Fusion proteins that contain natural junctions also provide advantages for use as in vivo therapeutic or diagnostic agents, because they have reduced potential for immunogenicity when the parental polypeptides are from the same species as the patient.

Conventional fusion proteins contain non-self sequences due to the juxtaposition of amino acid sequences from different parental proteins. These sequences do not occur naturally and can be immunogenic (e.g., form B cell epitopes, form T cell epitopes). Consequently, conventional fusion proteins can induce an immune response in patients. Immunogenicity is an important aspect that can limit or prevent in vivo use of fusion proteins. Immunogenicity occurs, for example, when epitopes on a recombinant fusion protein stimulate cellular (T cell) immune responses. T cell epitopes consist of linear peptides that are usually 8 to 11 amino acids in length. Thus, as described herein, recombinant fusion proteins can be designed and produced that have desired biological functions, but a reduced number of or no T epitopes in comparison to fusion proteins prepared using conventional methods.

In order to function as T cell epitopes, peptides derived from recombinant proteins must fulfill several requirements. They must survive intracellular proteolytic processing and must be able to bind to a host's major histocompatibility molecules (e.g., human HLA molecules). Another factor that influences whether a peptide is recognized as a T cell epitope is the extent of self. Importantly, T cells directed at epitopes belonging to self proteins are tolerized or eliminated during thymic development (See, e.g., Rosmalen et al., 2002). However, some auto-specific T cells persist in the periphery, where they are suppressed by CD4(+) CD25(+) regulatory T cells (See, e.g., Papiernik, 2001, and Shevach et al., 2001). When fusion proteins that contain T cell epitopes are administered tolerance can be breeched. In this situation, foreign and even self-peptides derived from a fusion protein can induce an immune response. It is therefore desirable to reduce the number of T cell epitopes in fusion proteins. As described herein, this can be accomplished by maximizing the extent of “self” within any given continuous peptide sequence found within a recombinant fusion protein.

Recombinant fusion proteins made up of two or more portions (e.g., domains) that do not occur next to one another in naturally occurring proteins, comprise junctions that connect the portions. Since the portions are not connected in their native context, such junctions commonly comprise a non-self amino acid sequence motif at the junction (the site where the switch occurs from one native peptide sequence to another). This type of junction includes two amino acids that are not normally adjacent within their native context. Therefore, a peptide spanning such a junction is a non-self peptide and has the potential to act as an epitope for T cells. Using the approach described herein, the junction is designed to reduce or eliminate the potential to act as an epitope for T cells. The approach described herein is illustrated conceptually in the following schemes in which a fusion protein is produced that contains a portion derived from hypothetical protein X and a portion derived from hypothetical protein Y.

Protein X has the following sequence:

. . . XXXXXX11111111111XXXXXXX2XXXXXXXXX-XXXXXXXX333X3XXXXXXXX . . .

Protein Y has the following sequence:

. . . YYYYYY11111111111YYYYYYY2YYYYYYYYY-YYYYYYYY333Y3YYYYYYYY . . .

In each of the conceptual protein sequences, “-” denotes the boundary between N-terminal and C-terminal domains within protein X an protein Y. A conventional fusion protein in which the amino terminal domain of protein X is fused to the carboxy-terminal domain of protein Y at the native domain boundary is illustrated in Scheme 1. This type of junction includes two amino acids that are not normally adjacent within their native context (x-y)t. Therefore, a peptide spanning such a junction is a non-self peptide and has the potential to act as an epitope for T cells.

Scheme 1
. . . XXXXXX11111111111XXXXXXX2XXXXXXXX-YYYYYYYYYY333Y3YYYYYYYY . . .

As shown in the Schemes 2-4, one application of the invention involves fusion proteins in which a domain from a first polypeptide is to be fused to a domain from a second polypeptide. To prevent the creation of potential new T-cell epitopes, the junction is moved away from the native domain boundary by one or more amino acids (either N-terminally or C-terminally) to an amino acid sequence motif that is conserved in both domains that are to be fused. Since the conserved amino acid motif representing the new fusion site is found in both parental domains, peptides that could be produced in vivo that span the new junction have fewer or no amino acids that are not normally adjacent in the parental proteins, and consequently have reduced potential to function as T cell epitopes.

Scheme 2
| ≧1aa |
. . . XXXXXX11111111111YYYYYYY2YYYYYYYY-YYYYYYYYYY333Y3YYYYYYYY . . .

For example, as illustrated in Scheme 2, a fusion protein comprising a domain from protein X and a domain from protein Y can be prepared. In this example, proteins X and Y each contain a conserved amino acid sequence motif (underlined). This shared motif is the fusion site, any peptide spanning the new domain fusion site that might potentially be a T cell epitope would be entirely self, with regard to the N-terminal domain and/or with regard to the C-terminal domain, thereby eliminating the possibility of being recognized as non-self by T cells.

Scheme 3
| ≧1aa |
. . . XXXXXX11111111111XXXXXXX2YYYYYYYY-YYYYYYYYYY333Y3YYYYYYYY . . .

In another example, the conserved amino acid motif representing the new domain fusion site could be 1 amino acid in length, so that any peptide spanning the boundaries of the two domains in the fusion protein that might potentially be a T cell epitope would not contain any amino acids that are not found adjacent in the native context of domain boundary in parental protein Y (Scheme 3).

Scheme 4
| ≧1aa |
. . . XXXXXX11111111111XXXXXXX2XXXXXXXX-XXXXXXXXXX333Y3YYYYYYYY . . .
or
. . . XXXXXX11111111111XXXXXXX2XXXXXXXX-XXXXXXXXXX333X3YYYYYYYY . . .

As shown in Scheme 4, in some examples, the conserved amino acid motif is 2-10 amino acids in length and the amino acid sequence of the conserved amino acid motif is not identical in the two parental polypeptides.

### Application to Fusion of a Vκ Domain to a CH1 Domain

Additionally, domain interactions are important for the integrity and function of many proteins, including proteins and fusion proteins that contain an immunoglobulin fold. For example, the interactions between immunoglobulin variable and constant domains play an important role in the structure of IgGs (See, e.g., Rothlisberger et al., 2005). To produce fusion proteins that contain immunoglobulin domains, or portions of immunoglobulin domains, it is important to take into consideration the protein-protein interactions that these domains participate in within their native context. For example, the interactions between Vκ and Cκ differ from those between VH and CH1 in immunoglobulins, suggesting that it is potentially problematic to generate Vκ-CH1 or VH-Cκ fusion proteins. However, for some applications it is desirable to generate IgG-like molecules comprising 4 Vκ variable domains, Fab′ fragments comprising 2 Vκ variable domains, or “inside-out” molecules similar to those described by Morrison et al. (1998) and Chan et al. (2004). Such work would require generating fusion proteins of Vκ and CH1 domains.

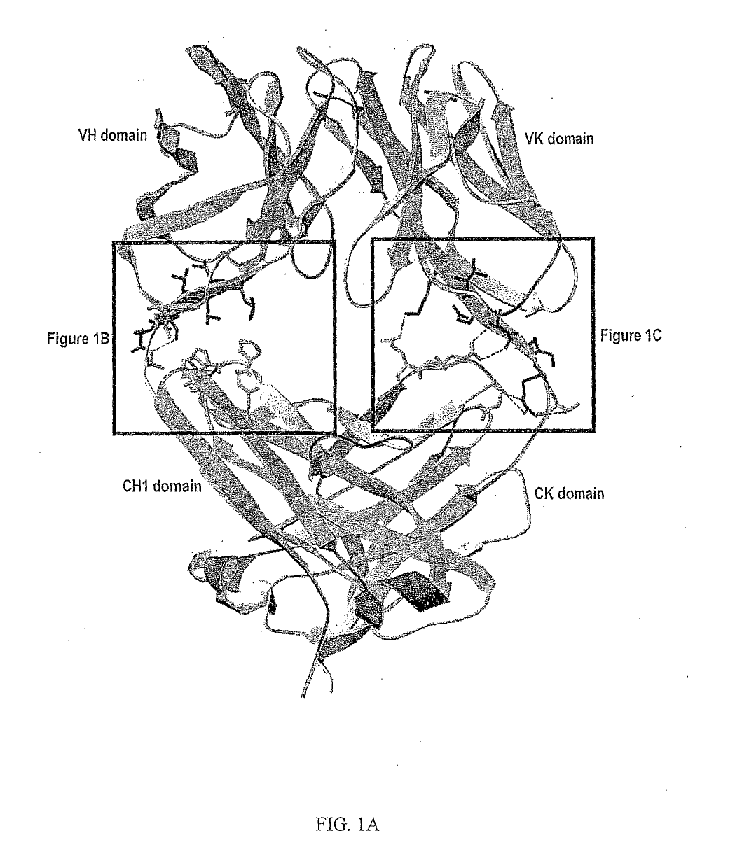

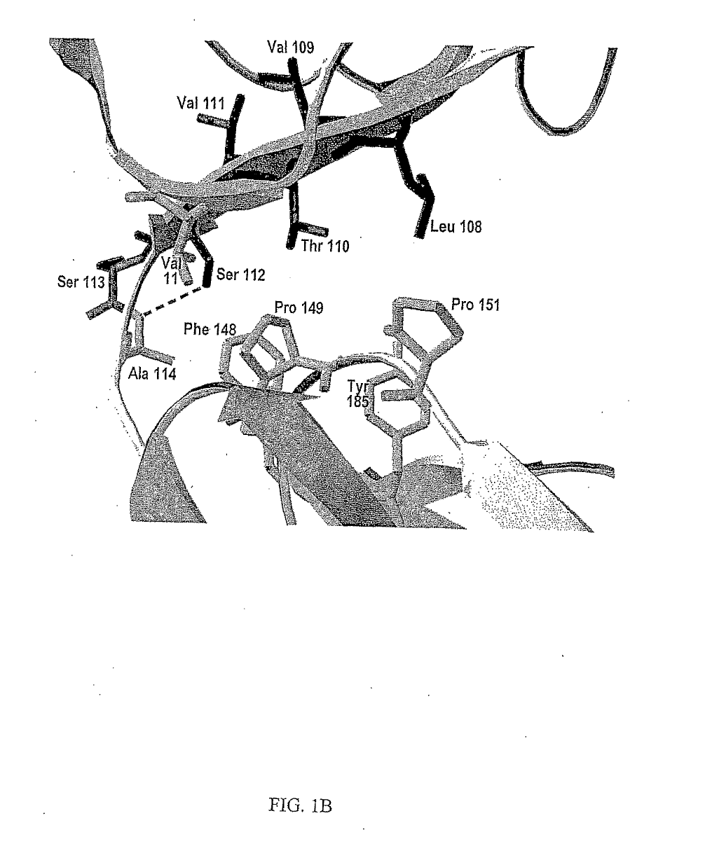

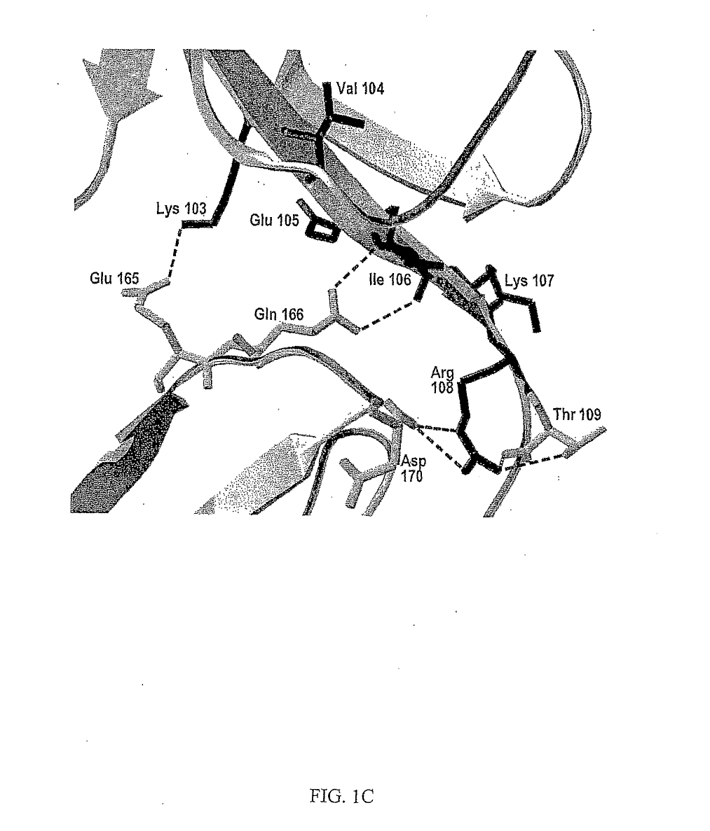

The structure of a typical human Fab′ fragment is shown in FIG. 1A, which represents the structure 1VGE, published by Chacko et al. (1996). Lesk and Chothia reported (1988) that the interactions between domains VH and CH1 are determined significantly by 3 highly conserved residues in VH (H11 [Leu or Val], H110 [Thr] and H112 [Ser]) and 2 highly conserved residues in CH1 (H148 [Phe] and H149 [Pro]). This cluster of 5 residues, illustrated in FIG. 1B, provides a degree of controlled flexibility (termed elbow motion) that changes the orientation of Vκ or V_{H} domains relative to Cκ or CH1 domains in immunoglobulins, respectively. This domain boundary can contribute to the functionality of some antibodies (Landolfi et al. 2001). In addition, the hydrophobic side chain of the conserved residue H108 (Leu) is located at the VH-CH1 interface and may participate in hydrophobic interactions between VH and CH1.

If a Vκ-CH1 fusion were prepared simply by joining an entire Vκ domain (up to residue L108 or L109) to a CH1 domain (from residue H114 [Ala]), 3 of the above 4 conserved residues that CH1 naturally interacts with would not be present in the new variable domain. Residue H11 (Leu/Val) is conserved between many VH and Vκ domains, but residues H108 (Leu), H110 (Thr) and H112 (Ser) are not. This would result in the loss of the conserved VH-CH1 domain interface at the variable domain constant domain boundary, in particular, the loss of hydrophobic interactions. Furthermore, this could result in the loss of a hydrogen bond that may exist between the side chain of residue H112 (Ser) and the backbone nitrogen of residue H114 (Ala), as it does in the example of the Fab structure 1VGE (FIG. 1A). In addition to the loss of residues that stabilize the VH-CH1 interface, new residues would be introduced that would potentially destabilize the fusion protein. Charged residues would be present in the C-terminal portion of the new variable domain, for example L103 (Lys), L107 (Lys) or L108 (Arg). These charged Vκ residues might cause repulsion between Vκ and CH1 at the variable domain-constant domain interface and prevent good domain packing.

Using Swiss-PdbViewer (version 3.7) and the GROMOS96 43B1 parameter set (van Gunsteren et al. 1996), it was determined that the C-terminal VH residues H108 to H113 (sequence LeuValThrValSerSer) in the human Fab structure 1VGE contribute −100.953 KJ/mol of to the total energy of the molecule. If these residues are replaced by the sequence LysValGluIleLysArg (a sequence commonly representing the C-terminal Vκ residues L103 to L108), the contribution to the total energy of the molecule would be +57.4 kJ/mol. This indicates that a Fab fragment could be significantly destabilized by replacing an entire VH domain with an entire Vκ domain which results in replacement of the C-terminal VH residues H108 to H113. Furthermore, any introduced charged Vκ residues would be prone to proteolysis in a context in which they are not accommodated by interactions with Cκ that they naturally participate in when found in their native context of a Vκ-Cκ junction.

In accordance with the invention, a Vκ-CH1 fusion protein can be generated by joining the N-terminal portion of a Vκ domain to the C-terminal portion of a VH domain in such a manner that the fusion site becomes the GlyXaaGlyThr (SEQ ID NO:386) motif that is conserved between Vκ (residues L99-L102) and VH (residues H104-H107). In this way, all 4 of the 4 conserved residues that CH1 naturally interacts with can be present in the new variable domain. Residue H11 (Leu/Val) is already conserved between many VH and Vκ domains, and residues H108 (Leu), H110 (Thr) and H112 (Ser) would also be present as the fusion site has been moved toward the N-terminus of Vκ, and residues H104 to H113 would be VH residues. This natural junction would preserve the VH-CH1 domain interface, including preservation of the elbow joint, and preservation of hydrophobic interactions and of hydrogen bonding, to a greater extent than if an entire Vκ domain (up to residue L108/L109) were simply joined to a CH1 domain (from residue H114). In addition, the natural junction would avoid the repulsion and susceptibility to proteolysis potentially caused by the presence of charged Vκ residues in the region L103-L108).

### Application to Fusion of a VH Domain to a Cκ Domain

Typical interactions found between Vκ and Cκ domains and also seen in 1VGE are highlighted in FIG. 1C. In particular, the Vκ-Cκ interface is stabilised by hydrogen bonding between the side chain of Vκ residue L103 (Lys) and Cκ residue L165 (Glu) and by hydrogen bonding between the side chain of Vκ residue L108 (Arg, in humans partially encoded by the Jκ exon and partially encoded by the Cκ exon) and Cκ residues L109 (Thr) and L170 (Asp). In addition, residue L106 (Ile) also participates, via its backbone nitrogen and oxygen, in hydrogen bonding with the side chain of Cκ residue L166 (Gln).

If a Vκ-CH1 fusion were prepared by simply joining an entire VH domain (up to residue H113 (Ser) to a Cκ domain (from residue L108 [Arg] or residue L 109 [Thr]), the above interactions would be lost (or could be modified, in the case of backbone interactions). Using Swiss-PdbViewer (version 3.7) and the GROMOS96 43B1 parameter set (van Gunsteren et al. 1996), it was determined that the C-terminal Vκ residues L103 to L108 (sequence LysValGluIleLysArg (SEQ ID NO:541)) in the human Fab structure 1VGE contributed −309.32 KJ/mol to the total energy of the molecule. If these residues are replaced by the sequence LeuValThrValSerSer (SEQ ID NO:421) (a sequence commonly representing the C-terminal V_{H} residues H108 to H113), the contribution to the total energy of the molecule would be −5.202 kJ/mol. This indicates that a Fab fragment could be significantly destabilised by replacing an entire Vκ domain with an entire VH domain, which would result in replacement of C-terminal Vκ residues L103 to L108.

In accordance with the invention, a VH-Cκ fusion protein can be generated by joining the N-terminal portion of the VH domain to the C-terminal portion of the Vκ domain in such a manner that the fusion site becomes the GlyXaaGlyThr (SEQ ID NO:386) motif that is conserved between Vκ (residues L99-L102) and VH (residues H104-H107). In this way, the residues that Cκ naturally interacts with can be present in the new variable domain. This natural domain junction should result in a fusion protein with significantly better properties than the fusion protein with an unnatural domain junction.

### Fusion Proteins

The fusion proteins of the invention comprise at least two portions derived from two different polypeptides, and at least one natural junction between the two portions. If desired, the fusion protein can contain three or more portions, and some of the junctions between portions can be non-natural. In one aspect, the recombinant fusion protein comprises a hybrid domain. The hybrid domain comprises a first portion (amino acid sequence) that is derived from a first polypeptide, a second portion (amino acid sequence) that is derived from a second polypeptide, and a conserved amino acid motif that is present in the first polypeptide and the second polypeptide. The first polypeptide will comprise a domain that has the formula (X1-Y-X2), and the second polypeptide will comprise a domain that has the formula (Z1-Y-Z2), and the fusion protein will comprise a hybrid domain that has the formula (X1-Y-Z2).

In the above formulae,

Y is a conserved amino acid motif;

X1 and Z1 are the amino acid motifs that are located adjacent to the amino-terminus of Y in the first polypeptide and the second polypeptide, respectively;

X2 and Z2 are the amino acid motifs that are located adjacent to the carboxy-terminus of Y in the first polypeptide and the second polypeptide, respectively;

with the proviso that when the amino acid sequences of X1 and Z1 are the same, the amino acid sequences of X2 and Z2 are not the same; and when the amino acid sequences of X2 and Z2 are the same, the amino acid sequences of X1 and Z1 are not the same.

The number of amino acids represented by X1, X2, Z1 and Z2 is dependent on the size of the hybrid domain, and the size of the domains in the parental polypeptides. Generally, X1, X2, Z1 and Z2 each, independently, consist of about 1 to about 400, about 1 to about 200, about 1 to about 100, about 1 to about 50, about 1 to about 40, about 1 to about 30, about 1 to about 20, about 1 to about 15, about 1 to about 10, about 1 to about 6, about 15, about 14, about 13, about 12, about 11, about 10, about 9, about 8, about 7, about 6, about 5, about 4, about 3, about 2 or 1 amino acid. Similarly, the size of the hybrid domain can vary, and is depend on the size of the domains that contain Y in the parental proteins. The overall size of the hybrid domain can be about 75 to about 400, about 75 to about 350, about 75 to about 300, about 75 to about 250, about 75 to about 150, about 75 to about 125, about 75 to about 100 or about 75 amino acids. In particular embodiments, the hybrid domain is about the size of an immunoglobulin variable domain or immunoglobulin constant domain. In some embodiments, the hybrid domain is about 1 kDa to about 25 kDa, about 5 kDa to about 25 kDa, about 5 kDa to about 20 kDa, about 5 kDa to about 15 kDa, about 6 kDa, about 7 kDa, about 8 kDa, about 9 kDa, about 10 kDa, about 11 kDa, about 12 kDa, about 13 kDa or about 14 kDa.

The conserved amino acid motif Y can consist of one to about 50 amino acid residues. In certain embodiments, Y consists of about 3 to about 50 amino acids, about 3 to about 40 amino acids, about 3 to about 30 amino acids, about 3 to about 20 amino acids, about 3 to about 15 amino acids, about 3 to about 14 amino acids, about 3 to about 13 amino acids, about 3 to about 12 amino acids, about 3 to about 11 amino acids, about 3 to about 10 amino acids, about 3 to about 9 amino acids, about 3 to about 8 amino acids, about 3 to about 7 amino acids, about 3 to about 6 amino acids, about 3 to about 5 amino acids, at least about 8 amino acids, up to about 11 amino acids, or about 8 to about 11 amino acids. In other embodiments, Y consists of about 1 to about 11 amino acids, about 15 amino acids, about 14 amino acids, about 13 amino acids, about 12 amino acids, about 11 amino acids, about 10 amino acids, about 9 amino acids, about 8 amino acids, about 7 amino acids, about 6 amino acids, about 5 amino acids, about 4 amino acids, about 3 amino acids, about 2 amino acids, or about 1 amino acid.

The conserved amino acid motif Y is found in two or more parental polypeptides, of which at least a portion is incorporated into a fusion protein of the invention. The fusion protein of the invention, and the hybrid domain in the fusion protein, can contain portions from any desired parental polypeptides provided that each parental protein contains a conserved amino acid motif. For example, the parental polypeptides can be unrelated (e.g., from different protein superfamilies) or related (e.g., from the same protein superfamily). In certain embodiments, the fusion protein and hybrid domain contains portions derived from parental polypeptides from the same protein superfamily, such as the immunoglobulin superfamily, the tumor necrosis factor (TNF) superfamily or the TNF receptor superfamily.

The parental proteins can be from the same species or from different species. For example, the parental polypeptides can independently be from a human (Homo sapiens), or from a non-human species such as mouse, chicken, pig, torafugu, frog, cow (e.g., Bos taurus), rat, shark (e.g., bull shark, sandbar shark, nurse shark, horned shark, spotted wobbegong shark), skate (e.g., clearnose skate, little skate), fish (e.g., atlantic salmon, channel catfish, lady fish, spotted ratfish, atlantic cod, chinese perch, rainbow trout, spotted wolf fish, zebrafish), possum, sheep, Camelid (e.g., llama, guanaco, alpaca, vicunas, dromedary camel, bactrian camel), rabbit, non-human primate (e.g., new world monkey, old world monkey, cynomolgus monkey (Macaca fascicularis), Callithricidae (e.g., marmosets)), or any other desired non-human species. In particular embodiments, both parental proteins are human, or one parental protein is human and the other is from a non-human species.

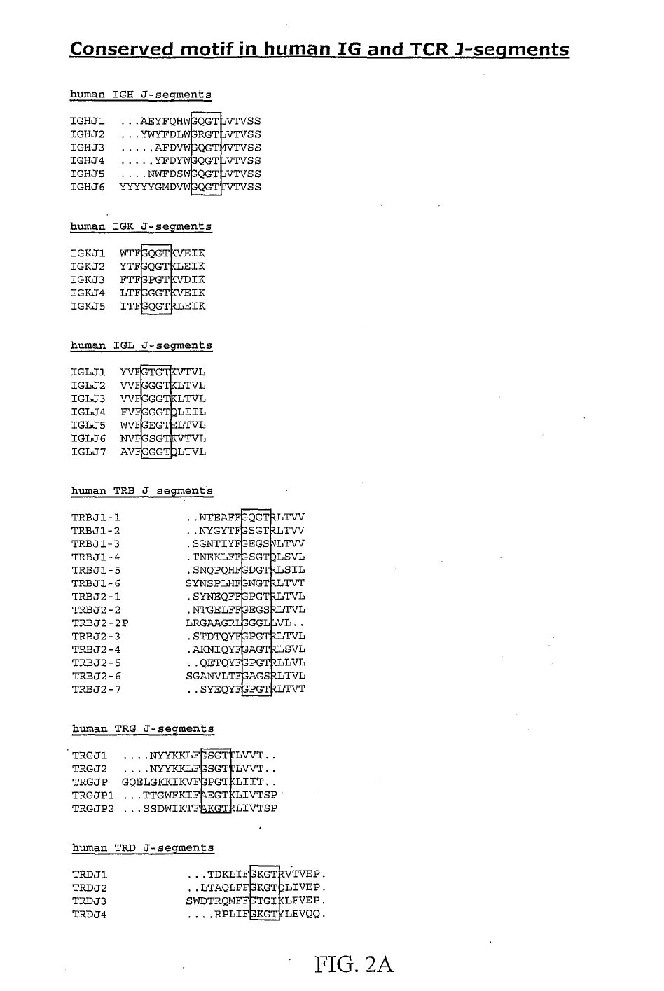

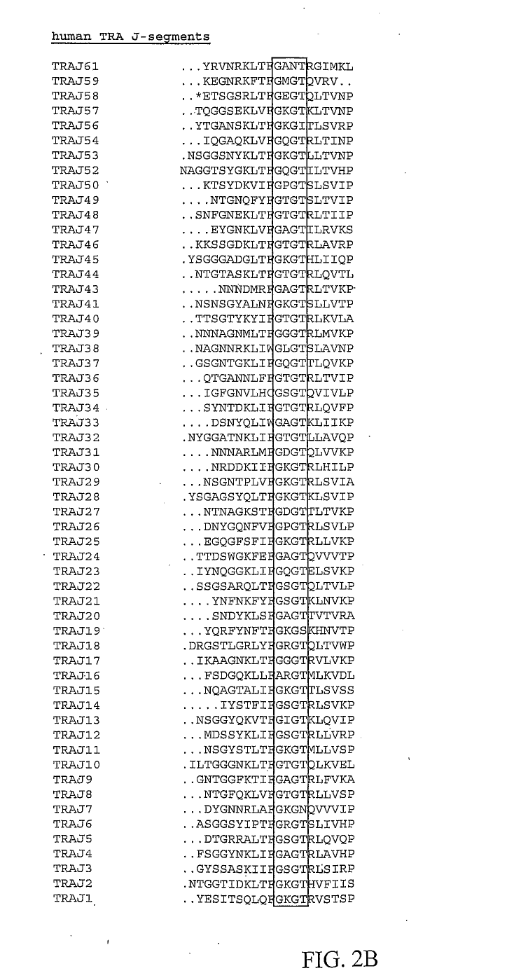

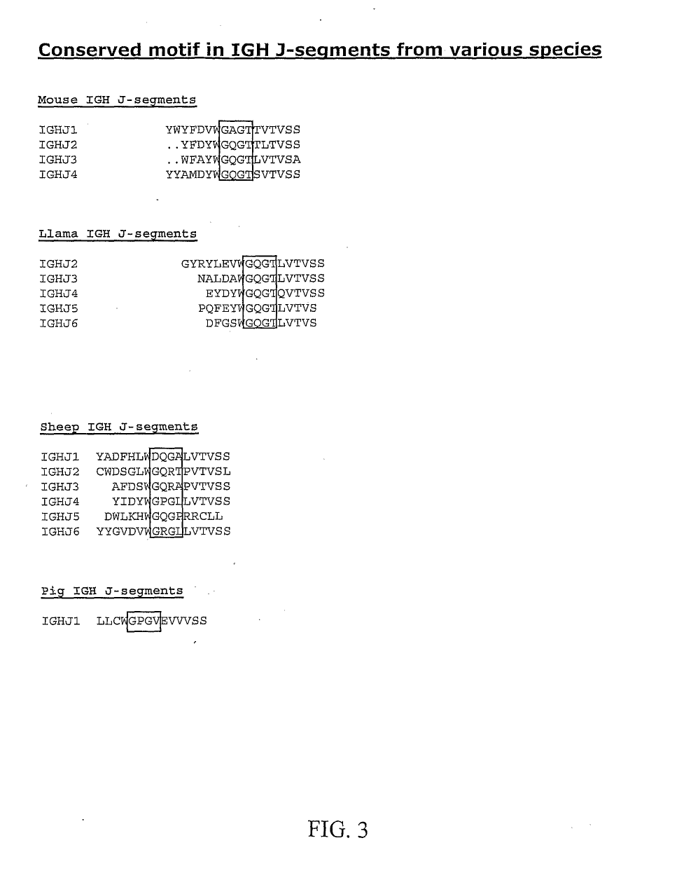

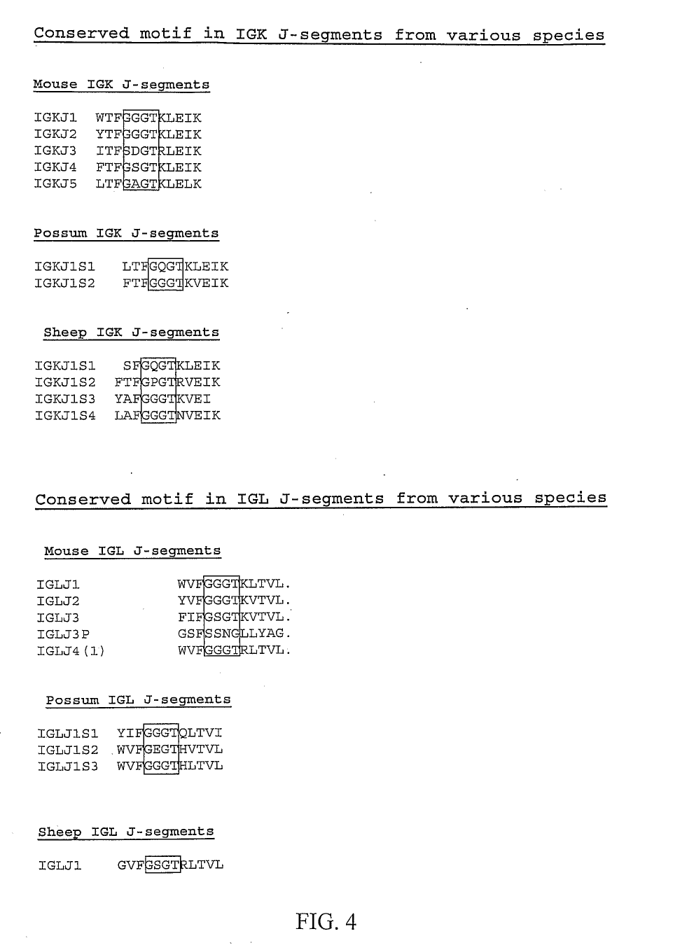

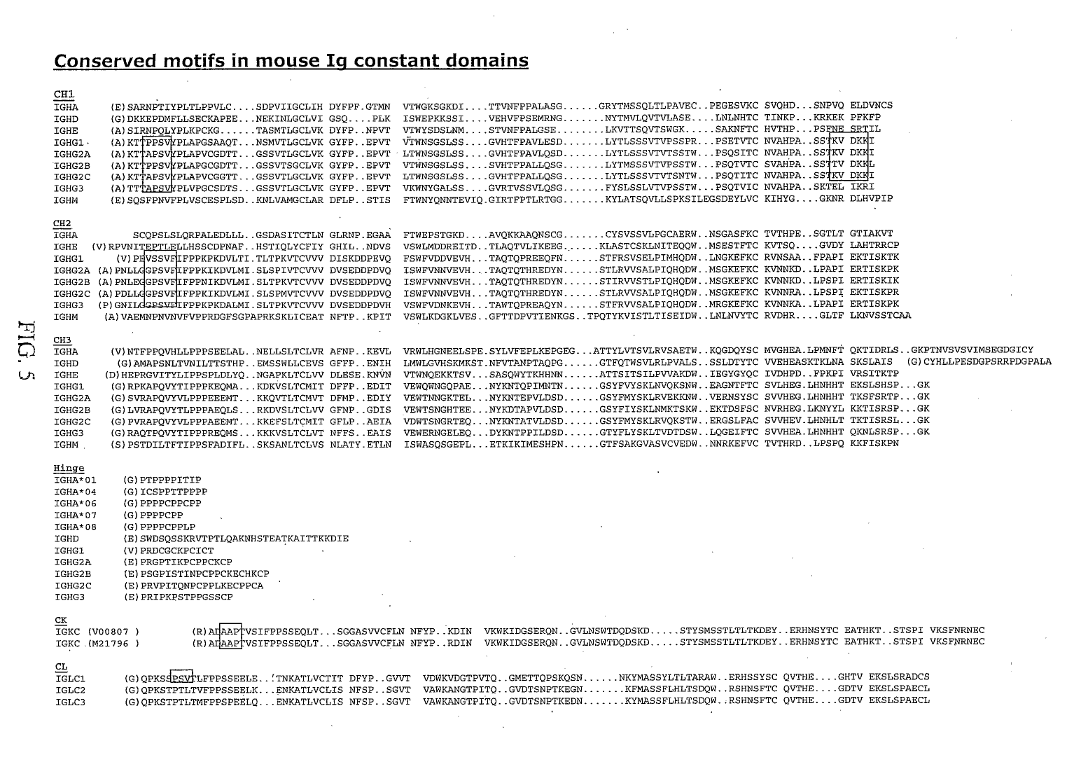

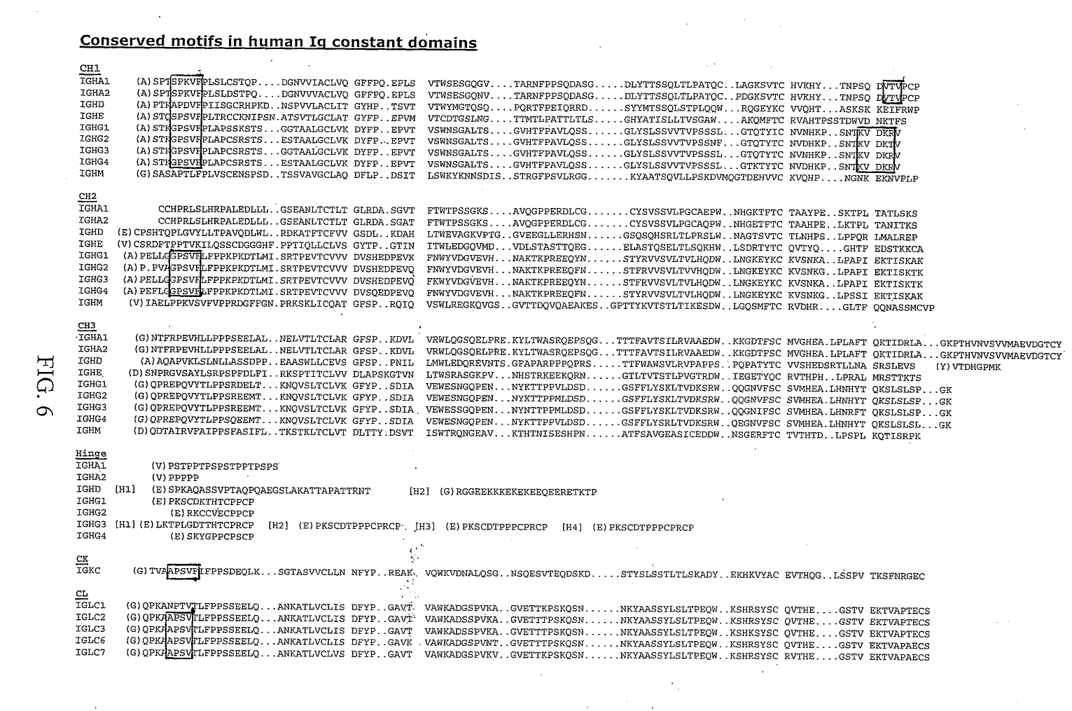

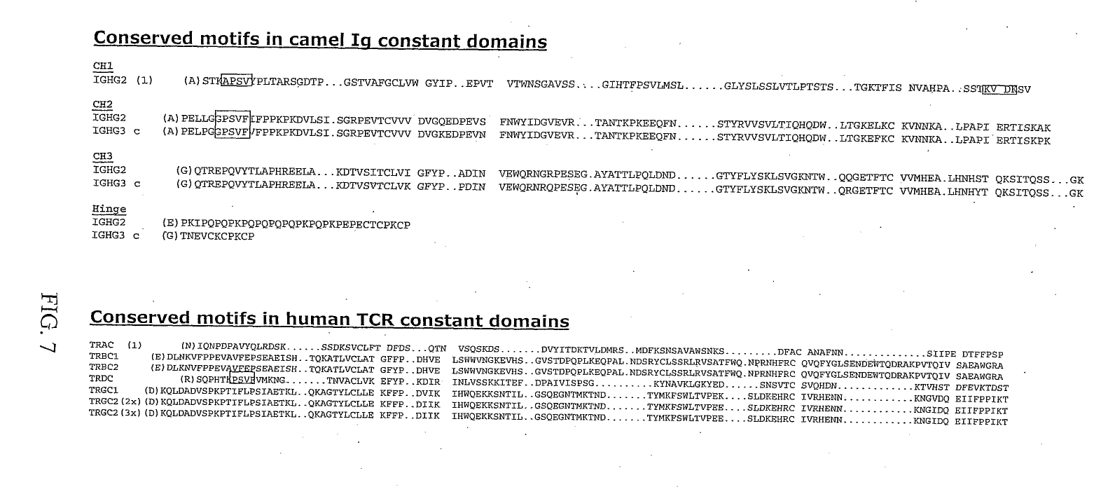

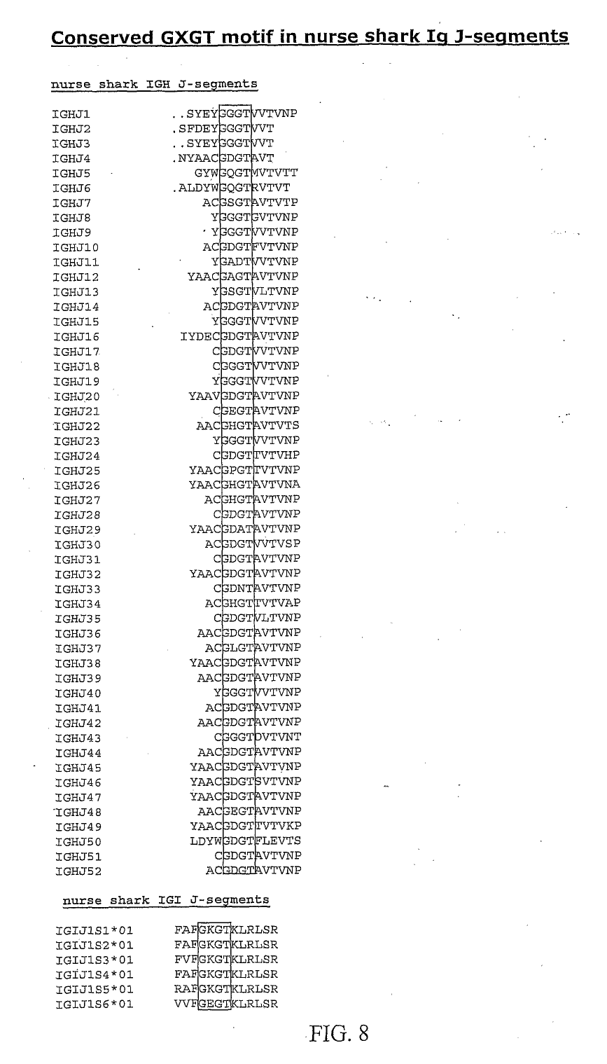

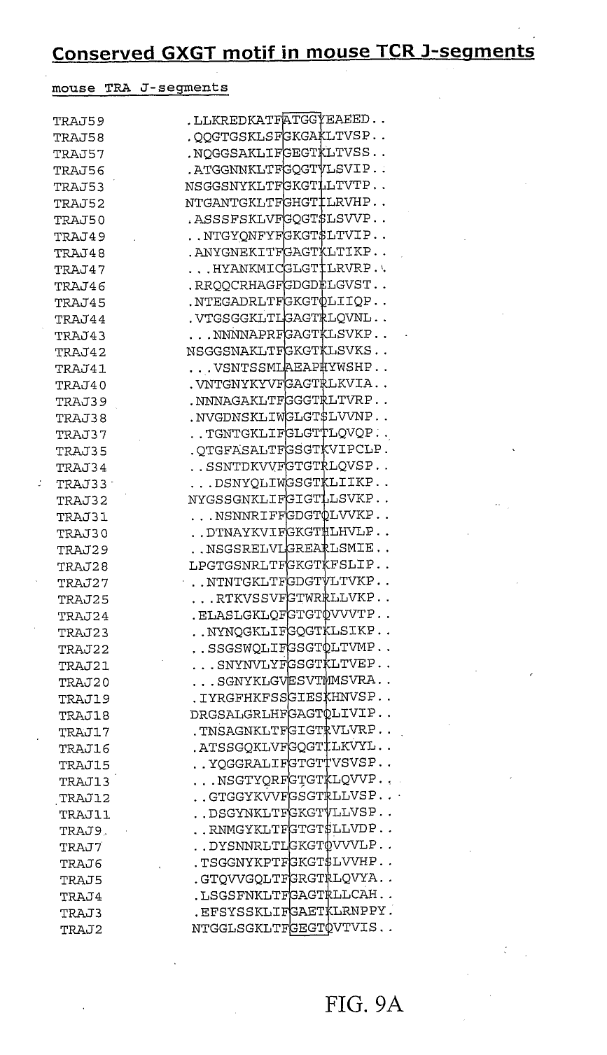

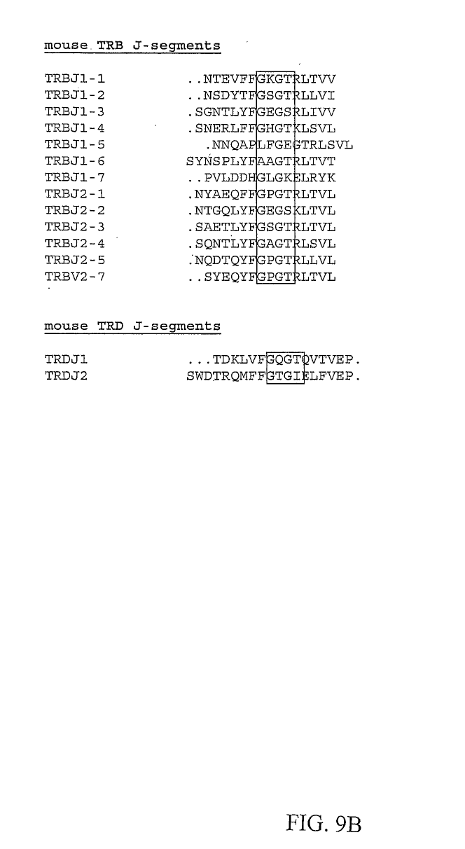

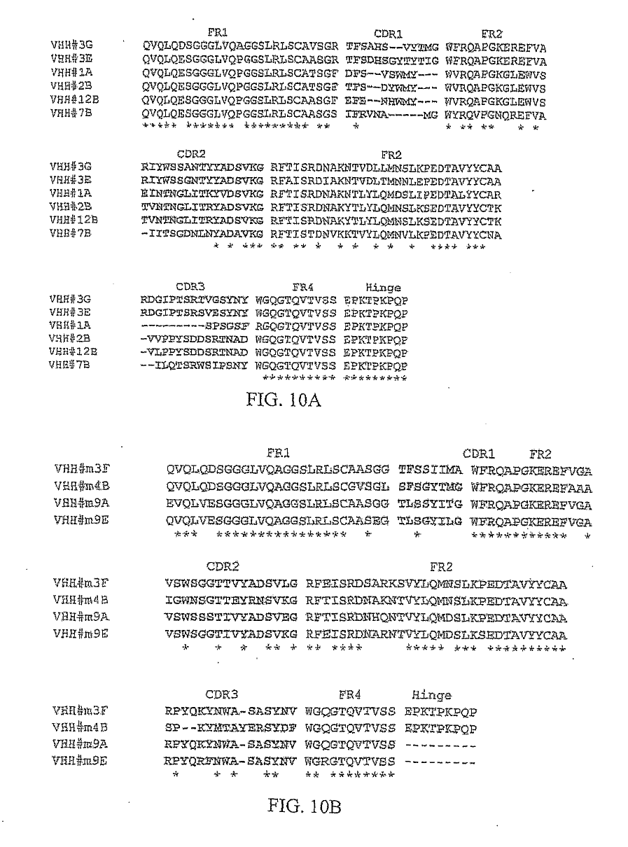

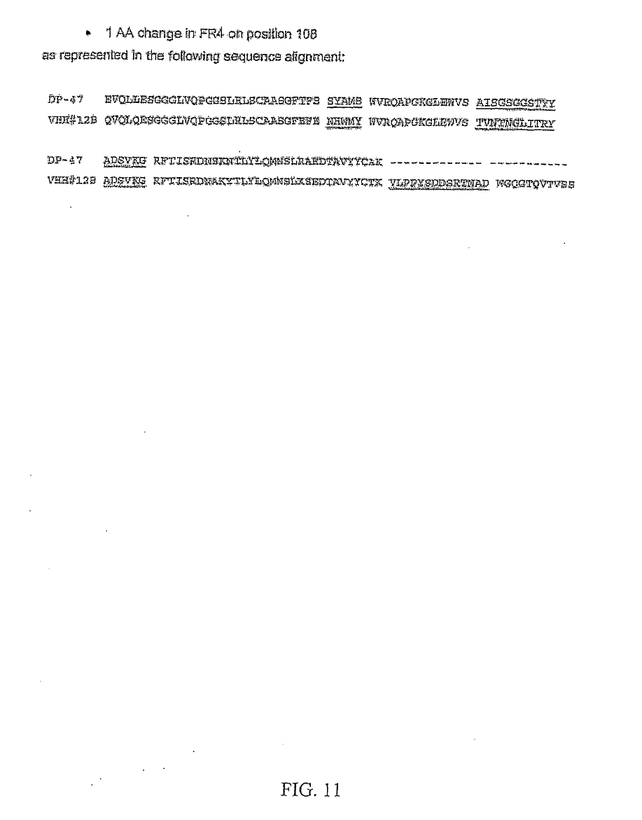

Conserved amino acid motifs can be readily identified using any suitable method, such as by aligning two or more amino acid sequences and identifying regions of conserved amino acid sequence. (See, e.g., FIGS. 2A and 2B) For example, as described herein, conserved amino acid motifs that are present in immunoglobulin proteins have been identified by alignment of immunoglobulin amino acid sequences. Particular examples of conserved amino acid motifs include: GlyXaaGlyThr (SEQ ID NO:386) or GlyXaaGlyThrXaa(Val/Leu) (SEQ ID NO:387) in framework region (FR) 4 of antibody variable domains; GluAspThrAla (SEQ ID NO:388), ValTyrTyrCys (SEQ ID NO:389), or GluAspThrAlaValTyrTyrCys (SEQ ID NO:390) in FR3 of antibody variable domains; (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393), or ValThrVal (SEQ ID NO:394) in antibody constant regions.

The hybrid domain in the fusion protein of the invention can be a hybrid immunoglobulin domain, such as a hybrid immunoglobulin variable domain or a hybrid immunoglobulin constant domain. For example, the fusion protein of the invention can comprise a hybrid T cell receptor variable domain or a hybrid antibody variable domain.

In some embodiments, the hybrid domain is a hybrid immunoglobulin variable domain (e.g., a hybrid antibody variable domain), and Y is located in a framework region (FR), such as FR1, FR2, FR3 or FR 4. In particular examples, Y is in FR4 and is GlyXaaGlyThr (SEQ ID NO:386) or GlyXaaGlyThrXaa(Val/Leu) (SEQ ID NO:387). For example, Y can be GlyXaaGlyThrXaaVal (SEQ ID NO:395) or GlyXaaGlyThrXaaLeu (SEQ ID NO:396). In these embodiments, X1 can be a portion of an antibody variable domain comprising FR1, complementarity determining region (CDR) 1, FR2, CDR2, FR3, and CDR3.

In other particular examples, the hybrid domain is a hybrid immunoglobulin variable domain (e.g., a hybrid antibody variable domain), Y is located in FR3 and is GluAspThrAla (SEQ ID NO:388), ValTyrTyrCys (SEQ ID NO:389), or GluAspThrAlaValTyrTyrCys (SEQ ID NO:390). In these embodiments, X1 can be a portion of an antibody variable domain comprising FR1, CDR1, FR2, and CDR2.

The hybrid domain in the fusion protein of the invention can be a hybrid a immunoglobulin constant domain, such as a hybrid T cell receptor constant domain or a hybrid antibody constant domain. In some embodiments, the hybrid domain is a hybrid immunoglobulin constant domain (e.g., a hybrid antibody constant domain), and Y is located in a constant domain, such as an antibody light chain constant domain (e.g., Cκ, Cλ), or an antibody heavy chain constant domain (e.g., CH1, hinge, CH2, CH3). For example, the hybrid domain can be a hybrid immunoglobulin CH1, CH2, Cκ or Cλ wherein Y is (Ser/Ala/Gly)Pro(Lys/Asp/Ser)Val(SEQ ID NO:391); a hybrid CH1, CH2, or Cκ wherein Y is (Ser/Ala/Gly)Pro(Lys/Asp/Ser)ValPhe (SEQ ID NO:392); a hybrid CH1 wherein Y is LysValAspLys(Ser/Arg/Thr) (SEQ ID NO:393) or ValThrVal (SEQ ID NO:394); or a hybrid TCR constant domain wherein Y is ProSerValPhe (SEQ ID NO:397). In particular embodiments of these examples, Y can be SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), or ValThrVal (SEQ ID NO:394).

The hybrid domain in the fusion protein of the invention can be bonded to an adjacent amino-terminal amino acid sequence, D, and/or be bonded to an adjacent carboxy-terminal amino acid sequence E, such that the recombinant fusion protein comprises a partial structure that has the formula

D-(X1-Y-Z2)-E,

wherein D is absent or is an amino acid sequence that is adjacent to the amino-terminus of (X1-Y-X2) in the first polypeptide, and E is absent or is an amino acid sequence that adjacent to the carboxy-terminus of (Z1-Y-Z2) in the second polypeptide.

For example, the fusion protein of the invention can comprise D-(X1-Y-Z2), wherein D is an immunoglobulin variable domain and (X1-Y-Z2) is a hybrid immunoglobulin constant domain. If desired, the fusion proteins can further comprise E and have the formula D-(X1-Y-Z2)-B, wherein D is an immunoglobulin variable domain, (X1-Y-Z2) is a hybrid immunoglobulin constant domain, and E is an immunoglobulin constant domain. As described above, the components of the fusion protein can be derived from parental proteins from any desired species. In this example of the fusion proteins of the invention, D can be an antibody variable region of non-human origin (e.g., from shark, mouse, Camelid), E can comprise a human immunoglobulin constant domain, and the hybrid constant domain (X1-Y-Z2) contains a portion (X1) of a non-human constant domain, a portion (Z2) of a human constant domain, and a conserved amino acid motif (Y) that is present in the non-human constant domain and the human constant domain. In other embodiments, D is absent and the fusion protein comprises a further domain that is amino terminal to (X1-Y-Z2). The further amino terminal domain can be bonded to (X1-Y-Z2) directly or indirectly through a natural junction or a non-natural junction.

In another example, the fusion protein of the invention comprises D-(X1-Y-Z2), wherein D is an immunoglobulin constant domain, and (X1-Y-Z2) is a hybrid immunoglobulin constant domain. If desired, the fusion protein of this example can contain additional components that are amino terminal to (X1-Y-Z2). For example, in one embodiment the fusion protein comprises an immunoglobulin variable domain, such as a V_{L}, VH or VHH, that is amino terminal to D. Thus, the fusion protein can have the structure: antibody variable domain-D-(X1-Y-Z2), wherein D is an immunoglobulin constant domain (e.g., an antibody constant domain), and (X1-Y-Z2) is a hybrid immunoglobulin constant domain (e.g., a hybrid antibody constant domain).

In another example, the fusion protein of the invention comprises (X1-Y-Z2)-E, wherein (X1-Y-Z2) is a hybrid immunoglobulin variable domain, and E is an immunoglobulin constant domain. If desired, the fusion protein of this example can contain additional components that are amino terminal to (X1-Y-Z2). For example, in one embodiment the fusion protein comprises another immunoglobulin variable domain, such as a V_{L}, VH or VHH, that is amino terminal to (X1-Y-Z2). Thus, the fusion protein can have the structure: antibody variable domain-(X1-Y-Z2)-E, wherein (X1-Y-Z2) is a hybrid immunoglobulin variable domain (e.g., a hybrid antibody variable domain) and E is an immunoglobulin constant domain (e.g., an antibody constant domain).

In another example, the fusion protein of the invention comprises (X1-Y-Z2)-E, wherein (X1-Y-Z2) is a hybrid immunoglobulin constant domain, and B is an immunoglobulin constant domain. If desired, the fusion proteins can contain additional components that are amino terminal to (X1-Y-Z2). For example, in one embodiment the fusion protein comprises an immunoglobulin variable domain, such as a V_{L}, VH or VHH, that is amino terminal to (X1-Y-Z2). Thus, the fusion protein can have the structure: antibody variable domain-(X1-Y-Z2)-E, wherein (X1-Y-Z2) is a hybrid immunoglobulin constant domain (e.g., a hybrid antibody CH1 domain) and E comprises an immunoglobulin constant domain (e.g., hinge, hinge-CH2, hinge-CH2-CH3).

Some of the fusion proteins of the invention comprises a hybrid immunoglobulin variable domain that is fused to an immunoglobulin constant domain, wherein said hybrid immunoglobulin variable domain comprises a hybrid framework region (FR) that comprises a portion from a first immunoglobulin FR from a first immunoglobulin and a portion from a second immunoglobulin FR from a second immunoglobulin, the first and second immunoglobulins each comprising a conserved amino acid motif. The hybrid FR has the formula

(F¹-Y-F²)

wherein Y is a conserved amino acid motif;

F¹ is the amino acid motif located adjacent to the amino-terminus of Y in the first immunoglobulin FR; and

F² is the amino acid motif located adjacent to the carboxy-terminus of Y in the second immunoglobulin FR.

The hybrid FR can be a hybrid FR1, hybrid FR2, hybrid FR3 or hybrid FR4. In one example, the first immunoglobulin is an antibody heavy chain, the second immunoglobulin is an antibody light chain, F¹ is derived from FR1, FR2, FR3 or FR4 of the antibody heavy chain variable domain, and F² is derived from the corresponding FR of the antibody light chain variable domain. Thus, the hybrid immunoglobulin domain can comprise FR1, CDR1, FR2, CDR2, FR3, CDR3 and a portion of FR4 (F¹) of an antibody heavy chain variable domain, a portion of FR4 (F²) of an antibody light chain variable domain, and a conserved amino acid motif (Y) that is present in FR4 of both the heavy chain and light chain variable domains. In other embodiments, the hybrid immunoglobulin domain can comprise FR1, CDR1, FR2, CDR2, and a portion of FR3 (F¹) of an antibody heavy chain variable domain, a portion of FR3, CDR3 and FR4 (F²) of an antibody light chain variable domain, and a conserved amino acid motif (Y) that is present in FR3 of both the heavy chain and light chain variable domains. Similarly, the hybrid immunoglobulin domain can comprise FR1, CDR1, and a portion of FR2 (F¹) of an antibody heavy chain variable domain, a portion of FR2 (F²), CDR2, FR3, CDR3 and FR4) of an antibody light chain variable domain, and a conserved amino acid motif (Y) that is present in FR2 both the heavy chain and light chain variable domains. The hybrid immunoglobulin domain can comprise a portion of FR1 (F¹) of an antibody heavy chain variable domain, a portion of FR1 (F²), CDR1, FR2, CDR2, FR3, CDR3 and FR4 of an antibody light chain variable domain, and a conserved amino acid motif (Y) that is present in FR1 both the heavy chain and light chain variable domains.

In another example, the first immunoglobulin is an antibody light chain, the second immunoglobulin is an antibody heavy chain, F¹ is derived from FR1, FR2, FR3 or FR4 of the antibody light chain variable region, and F² is derived from the corresponding FR of the antibody heavy chain variable region. Thus, the hybrid immunoglobulin domain can comprise FR1, CDR1, FR2, CDR2, FR3, CDR3 and a portion of FR4 (F¹) of an antibody light chain variable domain, a portion of FR4 (F²) of an antibody heavy chain variable domain, and a conserved amino acid motif (Y) that is present in FR4 both the light chain and heavy chain variable domains. In other embodiments, the hybrid immunoglobulin domain can comprise FR1, CDR1, FR2, CDR2, and a portion of FR3 (F¹) of an antibody light chain variable domain, a portion of FR3 (F²), CDR3 and FR4 of an antibody heavy chain variable domain, and a conserved amino acid motif (Y) that is present in FR3 both the light chain and heavy chain variable domains. Similarly, the hybrid immunoglobulin domain can comprise FR1, CDR1, and a portion of FR2 (F¹) of an antibody light chain variable domain, a portion of FR2 (F²), CDR2, FR3, CDR3 and FR4 of an antibody heavy chain variable domain, and a conserved amino acid motif (Y) that is present in FR2 both the light chain and heavy chain variable domains. The hybrid immunoglobulin domain can comprise a portion of FR1 (F¹) of an antibody light chain variable domain, a portion of FR1 (F²), CDR1, FR2, CDR2, FR3, CDR3 and FR4 of an antibody heavy chain variable domain, and a conserved amino acid motif (Y) that is present in FR1 both the light chain and heavy chain variable domains.

The hybrid immunoglobulin variable domain can be fused to any desired immunoglobulin constant domain. Generally, the carboxy-terminus of the hybrid immunoglobulin variable domain is fused directly to the amino terminus of an immunoglobulin constant domain. The fusion protein can comprise additional immunoglobulin constant domains and/or variable domains if desired. For example, a hybrid immunoglobulin variable domain can be fused to Cλ, Cκ, CH1, CH2, CH3, CH1-hinge-CH2-CH3, hinge-CH2-CH3, CH2-CH3, or a T cell receptor constant domain.

In preferred embodiments, the amino acid sequence F² is adjacent to the amino-terminus of the immunoglobulin constant domain to which the hybrid immunoglobulin variable domain is fused in a naturally occurring protein comprising said immunoglobulin constant domain. For example, when the second polypeptide is a TCR chain and F² is derived from a TCR FR4, the hybrid immunoglobulin domain is peptide bonded to the amino-terminus of a TCR constant domain. Similarly, when the second polypeptide is an antibody light chain and F² is derived from an antibody light chain variable region FR4, the hybrid immunoglobulin domain can be peptide bonded to the amino-terminus of an antibody light chain constant domain. In particular embodiments, the second polypeptide is a κ or λ light chain, F² is derived from a Vκ or Vλ FR4, and the hybrid immunoglobulin domain is bonded to the amino-terminus of Cκ or Cλ, respectively. When the second polypeptide is an antibody heavy chain and F² is derived from an antibody heavy chain variable domain FR4, the hybrid immunoglobulin domain can be bonded to the amino-terminus of an antibody heavy chain constant domain. In particular embodiments, the second polypeptide is an antibody heavy chain, F² is derived from an antibody heavy chain variable domain FR4 (e.g., V_{H} FR4, VHH FR4), and the hybrid immunoglobulin domain is bonded to the amino-terminus of CH1.

In particular embodiments, the hybrid immunoglobulin variable domain is a hybrid antibody variable domain and Y is GlyXaaGlyThr (SEQ ID NO:386) or GlyXaaGlyThrXaa(Val/Leu) (SEQ ID NO:387). For example, the fusion protein can comprise a hybrid antibody variable domain in which F¹ is Phe, Y is GlyXaaGlyThr (SEQ ID NO:386), and F² is (Leu/Met/Thr)ValThrValSerSer (SEQ ID NO:420). In particular embodiments, F² is LeuValThrValSerSer (SEQ ID NO:421), MetValThrValSerSer (SEQ ID NO:422), or ThrValThrValSerSer (SEQ ID NO:423). In other examples, the fusion protein can comprise a hybrid antibody variable domain, in which F¹ is Phe, Y is GlyXaaGlyThrXaa(Val/Leu) (SEQ ID NO:387), and F² is ThrValSerSer (SEQ ID NO:419). In particular embodiments, Y is GlyXaaGlyThrXaaVal (SEQ ID NO:395) or GlyXaaGlyThrXaaLeu (SEQ ID NO:396). Preferably the carboxy-terminus of these types of hybrid antibody variable domains is bonded directly to an antibody heavy chain constant domain, such as an IgG (e.g., IgG1, IgG2, IgG3, IgG4) constant domain. Preferably, the antibody heavy chain constant domain is a human antibody heavy chain constant domain. In particular embodiments, the carboxy-terminus of the hybrid antibody variable domain is bonded directly to IgG CH1 or IgG CH2 (e.g., IgG1 CH1, IgG4 CH1, IgG1 CH2, IgG4 CH2).

In other embodiments, the fusion protein comprises a hybrid variable domain in which F¹ is Trp, Y is GlyXaaGlyThr (SEQ ID NO:386), and F² is (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425). In particular embodiments, F² is LysValGluIleLys (SEQ ID NO:426), LysValAspIleLys (SEQ ID NO:427), LysLeuGluIleLys (SEQ ID NO:428), LysLeuAspIleLys (SEQ ID NO:429), ArgValGluIleLys (SEQ ID NO:430), ArgValAspIleLys (SEQ ID NO:431), ArgLeuGluIleLys (SEQ ID NO:432), ArgLeuAspIleLys (SEQ ID NO:433), LysValThrValLeu (SEQ ID NO:434), LysValThrIleLeu (SEQ ID NO:435), LysValIleValLeu (SEQ ID NO:436), LysValIleIleLeu (SEQ ID NO:437), LysLeuThrValLeu (SEQ ID NO:438), LysLeuThrIleLeu (SEQ ID NO:439), LysLeuIleValLeu (SEQ ID NO:440), LysLeuIleIleLeu (SEQ ID NO:441), GlnValThrValLeu (SEQ ID NO:442), GlnValThrIleLeu (SEQ ID NO:443), GlnValIleValLeu (SEQ ID NO:444), GlnValIleIleLeu (SEQ ID NO:445), GlnLeuThrValLeu (SEQ ID NO:446), GlnLeuThrIleLeu (SEQ ID NO:447), GlnLeuIleValLeu (SEQ ID NO:448), GlnLeuIleIleLeu (SEQ ID NO:449), GluValThrValLeu (SEQ ID NO:450), GluValThrIleLeu (SEQ ID NO:451), GluValIleValLeu (SEQ ID NO:452), GluValIleIleLeu (SEQ ID NO:453), GluLeuThrValLeu (SEQ ID NO:454), GluLeuThrIleLeu (SEQ ID NO:455), GluLeuIleValLeu (SEQ ID NO:456), or GluLeuIleIleLeu (SEQ ID NO:457).

In other examples, the fusion protein can comprise a hybrid antibody variable domain, in which F¹ is Trp, Y is GlyXaaGlyThrXaaVal (SEQ ID NO:395), and F² is (Glu/Asp)IleLys (SEQ ID NO:458) or (Thr/Ile)(Val/Ile)Leu (SEQ ID NO:459). In particular embodiments, F² is GluIleLys (SEQ ID NO:460), AspIleLys (SEQ ID NO:461), ThrValLeu (SEQ ID NO:462), ThrIleLeu (SEQ ID NO:463), IleValLeu (SEQ ID NO:464), or IleIleLeu (SEQ ID NO:465). Preferably the carboxy-terminus of these types of hybrid antibody variable domains is bonded directly to an antibody light chain constant domain, such as Cκ or Cλ. Preferably, the antibody light chain constant domain is a human antibody light chain constant domain.

In certain embodiments, the fusion protein that comprises a hybrid immunoglobulin variable domain that is fused to an immunoglobulin constant domain comprises a partial structure that has the formula (F¹-Y-F²)-Cκ, (F¹-Y-F²)-Cλ, (F¹-Y-F²)-CH1, (F¹-Y-²)-CH2 or (F¹-Y-F²)-Fc (e.g., F¹-Y-F²)-Fc-V, wherein the hybrid domain is a heavy chain V domain (e.g., human VH, VHH or camelized VH) and V is a heavy chain V domain (e.g., human VH, VHH or camelized VH), preferably both the hybrid domain and V are both human, both VHH or both camelized VH). The invention also provides dimers of such structures.

In certain embodiments, the fusion protein that comprises a hybrid immunoglobulin variable domain that is fused to an immunoglobulin constant domain further comprises a second immunoglobulin variable domain (e.g., antibody variable domain). The second immunoglobulin domain can be amino terminal or carboxy terminal to the hybrid immunoglobulin variable domain. Preferably, the second immunoglobulin variable domain is amino-terminal to the hybrid immunoglobulin variable domain in the fusion protein.

In some embodiments, the fusion protein of the invention comprises a non-human antibody variable region that is fused to a human antibody constant domain, wherein the non-human antibody variable region contains a hybrid FR4. The fusion protein contains a natural junction between the non-human antibody variable domain and the human antibody constant domain because the fusion site is in FR4 and not at the boundary between the variable domain and human constant domain. The hybrid FR4 has the formula (F¹-Y-F²).

In some embodiments, F¹ is Phe or Trp; Y is GlyXaaGlyThr (SEQ ID NO:386), and F² is (Leu/Met/Thr)ValThrSerSer (SEQ ID NO:420), (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425).

In other embodiments, F¹ is Phe or Trp, Y is GlyXaaGlyThrXaa(Val/Leu) (SEQ ID NO:387), and F² is ThrValSerSer (SEQ ID NO:419), (Glu/Asp)IleLys (SEQ ID NO:458) or (Thr/Ile)(Val/Ile)Leu (SEQ ID NO:459).

In some embodiments, the human antibody constant domain is a CH1 domain, Y is GlyXaaGlyThr (SEQ ID NO:386), and F² is (Leu/Met/Thr)ValThrValSerSer (SEQ ID NO:420). For example, in particular embodiments, F² is LeuValThrValSerSer (SEQ ID NO:421), MetValThrValSerSer (SEQ ID NO:422), or ThrValThrValSerSer (SEQ ID NO:423). In other embodiments, the human antibody constant domain is a CH1 domain, Y is GlyXaaGlyThrXaa(Val/Leu) (SEQ ID NO:387), and F² is ThrValSerSer (SEQ ID NO:418).

In some embodiments, the human antibody constant domain is a light chain constant domain, Y is GlyXaaGlyThr (SEQ ID NO:386), and F² is (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425). For example, in particular embodiments, F² is LysValGluIleLys (SEQ ID NO:426), LysValAspIleLys (SEQ ID NO:427), LysLeuGluIleLys (SEQ ID NO:428), LysLeuAspIleLys (SEQ ID NO:429), ArgValGluIleLys (SEQ ID NO:430), ArgValAspIleLys (SEQ ID NO:431), ArgLeuGluIleLys (SEQ ID NO:432), ArgLeuAspIleLys (SEQ ID NO:433), LysValThrValLeu (SEQ ID NO:434), LysValThrIleLeu (SEQ ID NO:435), LysValIleValLeu (SEQ ID NO:436), LysValIleIleLeu (SEQ ID NO:437), LysLeuThrValLeu (SEQ ID NO:438), LysLeuThrIleLeu (SEQ ID NO:439), LysLeuIleValLeu (SEQ ID NO:440), LysLeuIleIleLeu (SEQ ID NO:441), GlnValThrValLeu (SEQ ID NO:442), GlnValThrIleLeu (SEQ ID NO:443), GlnValIleValLeu (SEQ ID NO:444), GlnValIleIleLeu (SEQ ID NO:445), GlnLeuThrValLeu (SEQ ID NO:446), GlnLeuThrIleLeu (SEQ ID NO:447), GlnLeuIleValLeu (SEQ ID NO:448), GlnLeuIleIleLeu (SEQ ID NO:449), GluValThrValLeu (SEQ ID NO:450), GluValThrIleLeu (SEQ ID NO:451), GluValIleValLeu (SEQ ID NO:452), GluValIleIleLeu (SEQ ID NO:453), GluLeuThrValLeu (SEQ ID NO:454), GluLeuThrIleLeu (SEQ ID NO:455), GluLeuIleValLeu (SEQ ID NO:456), or GluLeuIleIleLeu (SEQ ID NO:457).

In other embodiments, the human antibody constant domain is a light chain constant domain, Y is GlyXaaGlyThrXaa(Val/Leu) (SEQ ID NO:387), and F² is (Glu/Asp)IleLys (SEQ ID NO:458) or (Thr/Ile)(Val/Ile)Leu (SEQ ID NO:459). For example, in particular embodiments, Y is GlyXaaGlyThrXaaVal (SEQ ID NO:395) or GlyXaaGlyThrXaaLeu (SEQ ID NO:396); and F² is GluIleLys (SEQ ID NO:460), AspIleLys (SEQ ID NO:461), ThrValLeu (SEQ ID NO:462), ThrIleLeu (SEQ ID NO:463), IleValLeu (SEQ ID NO:464), or IleIleLeu (SEQ ID NO:465).

Some of the fusion proteins of the invention comprise an immunoglobulin variable domain that is fused to a hybrid immunoglobulin constant domain, wherein said hybrid immunoglobulin constant domain comprises a portion from a first immunoglobulin constant domain and a portion from a second immunoglobulin constant domain, the first and second immunoglobulin constant domains each comprising a conserved amino acid motif. The hybrid immunoglobulin constant domain has the formula

(C¹-Y-C²)

wherein Y is a conserved amino acid motif;

C¹ is the amino acid motif located adjacent to the amino-terminus of Y in the first immunoglobulin constant domain; and

C² is the amino acid motif located adjacent to the carboxy-terminus of Y in the second immunoglobulin constant domain.

The hybrid immunoglobulin constant domain can comprise portions from any two immunoglobulin constant domains that contain a conserved amino acid motif. In certain embodiments, the hybrid immunoglobulin constant domain is a hybrid antibody constant domain that comprises a portion from a first antibody constant domain and a portion from a second antibody constant domain. For example, the hybrid antibody constant domain can be a hybrid CH1, hybrid hinge, hybrid CH2 or hybrid CH3, wherein portions of the hybrid domain are derived from antibody constant domains from different species (e.g., human and non-human, such as Camelid or nurse shark) or different isotypes (e.g., IgA, IgD, IgM, IgE, IgG (IgG1, IgG2, IgG3, IgG4)). The hybrid immunoglobulin constant domain can also comprise portions from two different constant domains, such as a portion from a CH1 domain and a portion from a CH2 domain.

In some embodiments, the hybrid antibody constant domain comprises portions that are derived from antibody constant domains of different species. For example, the first antibody constant domain can be a non-human antibody constant domain and the second antibody constant domain can be a human antibody constant domain. Suitable non-human antibody constant domains include those from mouse, chicken, pig, torafugu, frog, cow (e.g., Bos taurus), rat, shark (e.g., bull shark, sandbar shark, nurse shark, homed shark, spotted wobbegong shark), skate (e.g., clearnose skate, little skate), fish (e.g., atlantic salmon, channel catfish, lady fish, spotted ratfish, atlantic cod, chinese perch, rainbow trout, spotted wolf fish, zebrafish), possum, sheep, Camelid (e.g., llama, guanaco, alpaca, vicunas, dromedary camel, bactrian camel), rabbit, non-human primate (e.g., new world monkey, old world monkey, cynomolgus monkey (Macaca fascicularis), Callithricidae (e.g., marmosets)), or any other desired non-human species. Preferably, the amino terminus of a hybrid antibody constant domain is directly fused to the carboxy-terminus of an antibody variable domain that is from the same species as the amino terminal C¹ of the hybrid antibody constant domain. Preferably, the carboxy-terminal C² of the hybrid antibody constant domain is derived from a human antibody constant domain. For example, the fusion protein can comprise a partial structure having the formula: non-human V domain-(C¹-Y-C²), wherein C¹ is derived from a non-human constant domain (e.g., Cκ, Cλ, CH1) from the same species as the non-human V domain, Y is a conserved amino acid motif, and C² is derived from a human antibody constant domain.

In some embodiments, the hybrid antibody constant domain comprises a portion from a first antibody constant domain and a portion from a second antibody constant domain that are from antibodies of different isotypes. For example, in this type of the hybrid antibody constant domain, C¹ is a portion from an IgA, IgD, IgM, IgE, or IgG (e.g., IgG1, IgG2, IgG3, IgG4), and C² is a portion from an antibody constant domain of a different isotype than C¹. Preferably, C² is a portion from an IgG (e.g., IgG1, IgG2, IgG3, IgG4) constant domain. In a particular embodiment, the hybrid antibody constant domain comprises a portion from an IgG1 constant domain and a portion from an IgG4 constant domain. In such embodiments, C¹ is from an IgG1 constant domain and C² is from and IgG4 constant domain, or C² is from and IgG4 constant domain and C² is from an IgG1 constant domain.

In some embodiments, the hybrid immunoglobulin constant domain comprises a portion from a first antibody constant domain that is a light chain constant domain, and a portion from a second antibody constant domain that is a heavy chain constant domain. For example, the fusion protein can comprise a light chain antibody variable domain that is fused directly to a hybrid antibody constant domain, wherein the first antibody constant domain is a light chain constant domain and C¹ is derived from said light chain constant domain, the second antibody constant domain is a heavy chain constant domain and C² is derived from said heavy chain constant domain. For example, C² can be derived from an IgG (e.g., IgG1, IgG2, IgG3, IgG4) constant domain, such as an IgG CH1 (e.g., IgG1 CH1, IgG4 CH1), IgG hinge (e.g., IgG1 hinge, IgG4 hinge), IgG CH2 (e.g., IgG1 CH2, IgG4 CH2), IgG CH3 (e.g., IgG1 CH3 or IgG4 CH3).

In other embodiments, the hybrid immunoglobulin constant domain comprises a portion from a first antibody constant domain that is a heavy chain constant domain, and a portion from a second antibody constant domain that is a light chain constant domain. For example, the fusion protein can comprise a heavy chain antibody variable domain that is fused directly to a hybrid antibody constant domain, wherein the first antibody constant domain is a heavy chain constant domain and C¹ is derived from said heavy chain constant domain, and the second antibody constant domain is a light chain constant domain and C² is derived from said light chain constant domain. In particular embodiments, the first antibody constant domain is a CH1 domain and C¹ is derived from said CH1 domain.

In particular embodiments, the hybrid immunoglobulin constant domain comprises a portion from a first antibody constant domain that is a Camelid heavy chain constant domain, and a portion from a second antibody constant domain that is a heavy chain constant domain. For example, in some embodiments, the carboxy-terminal (C²) of the hybrid antibody constant domain is derived from a human heavy chain constant domain. If desired, the fusion protein can comprise a Camelid V_{HH} that is amino-terminal to the hybrid antibody constant domain. For example, in some embodiments, the fusion protein comprises a partial structure having the formula: Camelid V_{HH}-(C¹-Y-C²), wherein C¹ is derived from a Camelid heavy chain constant domain (e.g., Camelid CH1), Y is a conserved amino acid motif, and C² is derived from an antibody heavy chain constant domain (e.g., a human antibody constant domain, such as human CH1).

Some of fusion proteins of the invention comprise an immunoglobulin variable domain (e.g., antibody variable domain) that is fused directly to a hybrid antibody constant domain, wherein said hybrid antibody constant domain comprises a portion from a first antibody constant domain and a portion from a second antibody constant domain, the first and second antibody constant domains each comprising a conserved amino acid motif. The hybrid antibody constant domain has the formula

(C¹-Y-C²)

wherein Y is a conserved amino acid motif;

C¹ is the amino acid motif located adjacent to the amino-terminus of Y in the first antibody constant domain; and

C² is the amino acid motif located adjacent to the carboxy-terminus of Y in the second antibody constant domain. Preferably, the immunoglobulin variable domain is located amino-terminally to the hybrid antibody constant domain such that the fusion protein comprises a partial structure having the formula: antibody variable domain-(C¹-Y-C²).

In some embodiments, Y is (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393), or ValThrVal (SEQ ID NO:394). For example, in particular embodiments, Y is SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), or ValThrVal(SEQ ID NO:394). Preferably, the second antibody constant domain is a human antibody constant domain, and C² is derived from said human antibody constant domain. For example, the human antibody constant domain can be a human Cκ, a human Cλ or a human heavy chain constant domain, such as a human CH1, a human hinge, a human CH2 or a human CH3. In particular preferred embodiments, the human antibody constant domain is an IgG CH1 (e.g., IgG1 CH1, IgG4 CH1), IgG hinge (e.g., IgG1 hinge, IgG4 hinge), IgG CH2 (e.g., IgG1 CH2, IgG4 CH2), or IgG CH3 (e.g., IgG1 CH3 or IgG4 CH3), and C² is derived from said human antibody constant domain.

In particular embodiments, the fusion protein comprises an antibody light chain variable domain, such as a human light chain variable domain, that is fused to a hybrid antibody CH1 domain, wherein C¹ is GlnProLysAla (SEQ ID NO:466) or ThrValAla (SEQ ID NO:467), and Y is (Ala/Gly)ProSerVal (SEQ ID NO:468). In these embodiments, C² is the amino acid sequence that is adjacent to carboxy-terminus of Y in IgG CH1, such as human IgG CH1 (e.g., IgG1 CH1, IgG4 CH1).

In particular embodiments, the fusion protein comprises an antibody light chain variable domain, such as a human light chain variable domain, that is fused to a hybrid antibody CH2 domain, wherein C¹ is GlnProLysAla (SEQ ID NO:466) or ThrValAla (SEQ ID NO:467), and Y is (Ala/Gly)ProSerVal (SEQ ID NO:468). In these embodiments, C² is the amino acid sequence that is adjacent to carboxy-terminus of Y in IgG CH2, such as human IgG CH2 (e.g., IgG1 CH2, IgG4 CH2).

In particular embodiments, the fusion protein comprises an antibody heavy chain variable domain, such as a human heavy chain variable domain, that is fused to a hybrid antibody CH2 domain, wherein C¹ is SerThrLys (SEQ ID NO:469), and Y is (Ala/Gly)ProSerValPhe (SEQ ID NO:470). In these embodiments, C² is the amino acid sequence that is adjacent to the carboxy-terminus of Y in IgG CH2, such as human IgG CH2 (e.g., IgG1 CH2, IgG4 CH2).

In particular embodiments, the fusion protein comprises an antibody light chain variable domain, such as a human λ chain variable domain, that is fused to a hybrid antibody Cκdomain, wherein C¹ is GlnProLysAla (SEQ ID NO:466), and Y is (Ala/Gly)ProSerValPhe (SEQ ID NO:470). In these embodiments, C² is the amino acid sequence that is adjacent to the carboxy-terminus of Y in Cκ, such as human Cκ.

In particular embodiments, the fusion protein comprises an antibody heavy chain variable domain, such as a human heavy chain variable domain, that is fused to a hybrid antibody Cκ domain, wherein C¹ is SerThrLys (SEQ ID NO:469), and Y is (Ala/Gly)ProSerValPhe (SEQ ID NO:470). In these embodiments, C² is the amino acid sequence that is adjacent to the carboxy-terminus of Y in Cκ, such as human Cκ.

In particular embodiments, the fusion protein comprises an antibody light chain variable domain, such as a human κ chain variable domain, that is fused to a hybrid antibody Cλdomain, wherein C¹ is ThrValAla (SEQ ID NO:467), and Y is (Ala/Gly)ProSerVal (SEQ ID NO:468). In these embodiments, C² is the amino acid sequence that is adjacent to the carboxy-terminus of Y in Cλ, such as human Cλ.

In particular embodiments, the fusion protein comprises an antibody heavy chain variable domain, such as a human heavy chain variable domain, that is fused to a hybrid antibody Cλdomain, wherein C¹ is SerThrLys (SEQ ID NO:469), and Y is (Ala/Gly)ProSerVal (SEQ ID NO:468). In these embodiments, C² is the amino acid sequence that is adjacent to the carboxy-terminus of Y in Cλ, such as human Cλ.

In another aspect, the first portion and the second portion of the recombinant fusion protein of the invention are fused through a linker. The linker can be selected or designed to provide a natural junction between the first portion and the linker, the second portion and the linker or both the first and second portions and the linker. For example, when it is desired that a fusion protein of the invention contain portion (A) from a first polypeptide and portion (B) from a second polypeptide, the fusion protein can comprise a partial structure having the formula (A)-linker-(B), wherein a natural junction exists between (A) and the linker, between the linker and (B), or between (A) and the linker and the linker and (B). When a portion of a polypeptide that is to be included in a fusion protein of the invention is a domain, the linker used in the fusion protein can consist of the one to about 50 contiguous amino acids that are adjacent to the domain in a naturally occurring polypeptide that contains the domain. For example, the linker can consist of 1 to about 40, 1 to about 30, 1 to about 20, 1 to about 15, 1 to about 10, 1 to about 5, about 20, about 19, about 18, about 17, about 16, about 15, about 14, about 13, about 12, about 11, about 10, about 9, about 8, about 7, about 6, about 5, about 4, about 3, about 2 or about 1 amino acids that are adjacent to the domain in a naturally occurring polypeptide that contains the domain. This approach results in improved preservation of domain interactions in the fusion protein, thereby improving stability of the fusion protein.

In this aspect, the fusion protein generally comprises a first portion derived from a first polypeptide and a second portion derived from a second polypeptide, wherein said first polypeptide comprises a structure having the formula (A)-L1, wherein (A) is an amino acid sequence present in said first polypeptide; and L1 is an amino acid motif comprising 1 to about 50 amino acids that are adjacent to the carboxy-terminus of (A) in said first polypeptide. The fusion protein has the formula

(A)-L1-(B);

wherein (A) is the portion derived from the first polypeptide; L1 is an amino acid motif comprising 1 to about 50 contiguous amino acids that are adjacent to the carboxy-terminus of (A) in said first polypeptide and provides a linker that connects (A) and (B), and (B) is the portion derived from the second polypeptide. Preferably, (A) is a domain derived from the first polypeptide.

In some embodiments, the first polypeptide comprises (A) and the second polypeptide comprises a structure having the formula L1-(B) wherein L1 is an amino acid motif comprising 1 to about 50 amino acids that are adjacent to the amino-terminus of (B) in the second polypeptide. The fusion protein has the formula

(A)-L1-(B);

wherein (A) is the portion derived from the first polypeptide; L1 is an amino acid motif comprising 1 to about 50 contiguous amino acids that are adjacent to the amino-terminus of (B) in said second polypeptide and provides a linker that connects (A) and (B), and (B) is the portion derived from the second polypeptide. Preferably (B) is a domain derived from the second polypeptide.

In preferred embodiments, this aspect includes the proviso that at least one of (A) and (B) is a domain (e.g., (A) is a domain, (B) is a domain, (A) and (B) are both a domain). In other preferred embodiments, this aspect includes the further proviso that when (A) and (B) are both antibody variable domains 1) (A) and (B) are each human antibody variable domains; 2) (A) and (B) are each antibody heavy chain variable domains; 3) (A) and (B) are each antibody light chain variable domains; 4) (A) is an antibody light chain variable domain and (B) is an antibody heavy chain variable domain (e.g, VHH or VH); or 5) (A) is a VHH and (B) is an antibody light chain variable domain. Additionally or alternatively, preferred embodiments of this aspect include the proviso that when (A) is a V_{H} and (B) is a V_{L}, L1 does not consist of one to five or one to six contiguous amino acids from the amino-terminus of CH1. Additionally or alternatively, when (A) and (B) are both antibody variable domains the following is excluded from the invention, (A)-L1-(B) where (A) is a mouse VH, (B) is a mouse VL and L1 is SerAlaLysThrThrPro (SEQ ID NO:537), SerAlaLysThrThrProLysLeuGlyGly (SEQ ID NO:538), AlaLysThrThrProLysLeuGluGluGlyGluPheSerGluAlaArgVal (SEQ ID NO:539), or AlaLysThrThrProLysLeuGluGlu (SEQ ID NO:540). Additionally or alternatively, (A)-L1-(B) is not a fusion protein wherein (A) is a mouse VH, (B) is a mouse VL and L1 is a linker as disclosed in Le Gall et al., Protein Engineering, Design & Selection, 17:357-366 (2004), Kipriyanov et al., Int. J. Cancer, 77:763-772 (1998); Le Gall et al., J. Immunol. Methods, 285:111-127 (2004); Le Gall et al., FEBS Letters, 453:164-168 (1995); or Kipriyanov et al., Protein Engineering, 10:445-453 (1997).

In particular embodiments, the first polypeptide comprises (A)-L1, and the fusion protein comprises (A)-L1-(B), wherein (A) consists of complementarity determining region (CDR) 3, and L1 consists of framework 4. In other embodiments (A) comprises CDR1 and L1 comprises FR2; (A) comprises CDR2 and L1 comprises FR3; (A) comprises CDR1 and CDR2 (e.g., CDR1-FR2-CDR2) and L1 comprises FR3; (A) comprises CDR2 and CDR3 and L1 comprises FR4; or (A) comprises CDR1, CDR2 and CDR3 (e.g., CDR1-FR2-CDR2-FR3-CDR3) and L1 comprises FR4.

In other embodiments, the first polypeptide comprises (A), the second polypeptide comprises L1-(B) and the fusion protein comprises (A)-L1-(B), wherein (B) consists of CDR 3, and L1 consists of framework 3. In other embodiments (B) comprises CDR1 and L1 comprises FR1; (B) comprises CDR2 and L1 comprises FR2; (B) comprises CDR1 and CDR2 (e.g., CDR1-FR2-CDR2) and L1 comprises FR1; (B) comprises CDR2 and CDR3 and L1 comprises FR2; or (B) comprises CDR1, CDR2 and CDR3 (e.g., CDR1-FR2-CDR2-FR3-CDR3) and L1-comprises FR1.

In some embodiments, (A) is an immunoglobulin variable domain, such as an antibody variable domain. For example, (A) can be an antibody light chain variable domain (e.g., Cκ, Cλ) or an antibody heavy chain variable domain (e.g., V_{H}, V_{HH}). In such embodiments, L1 is 1 to about 50 contiguous amino acids that are adjacent to the carboxy-terminus of (A) in a naturally occurring polypeptide that comprises the variable domain A. For example, when (A) is Vκ (e.g., human Vκ), L1 is 1 to about 50 contiguous N-terminal amino acids of Cκ (e.g., human Cκ); when (A) is Vλ (e.g., human Vλ), L1 is 1 to about 50 contiguous N-terminal amino acids of Cλ (e.g., human Cλ), and when (A) is a heavy chain variable domain (e.g., human V_{H}, Camelid V_{HH}), L1 is 1 to about 50 contiguous N-terminal amino acids of CH1 (e.g., human CH1, Camelid V_{HH}). In some embodiments, (A) is a VH and L1 comprises the first 3 to about 12 N-terminal amino acids of CH1; (A) is a Vκ and l1 comprises the first 3 to about 12 N-terminal amino acids of Cκ; or (A) is a Vλ and L1 comprises the first 3 to about 12 N-terminal amino acids of Cλ.

In some embodiments, the second polypeptide comprises an immunoglobulin constant region, and (B) is derived from the immunoglobulin constant region. For example, (B) can comprise at least a portion of an antibody CH1, at least a portion of an antibody hinge, at least a portion of an antibody CH2, or at least a portion of an antibody CH3.

In some embodiments, (A) is an antibody variable domain, and (B) is an antibody variable domain. In these embodiments, the antibody variable domains (A) and (B) can be the same or different. For example, (A) can be an antibody heavy chain variable domain and (B) can be the same or a different antibody heavy chain variable domain; A) can be an antibody light chain variable domain and (B) can be the same or a different antibody light chain variable domain; A) can be an antibody heavy chain variable domain and (B) can be an antibody light chain variable domain, or A) can be an antibody light chain variable domain and (B) can be an antibody heavy chain variable domain. In exemplary embodiments (A) is a Vκ and (B) is a Vκ; (A) is a Vκ and (B) is a Vλ; (A) is a Vκ and (B) is a VH or a VHH; (A) is a Vλ and (B) is a Vκ; (A) is a Vλ and (B) is a Vλ; or (A) is a Vλ and (B) is a VH or a VHH. In preferred embodiments, this aspect additional or alternatively includes the proviso that when (A) and (B) are both antibody variable domains 1) (A) and (B) are each human antibody variable domains; 2) (A) and (B) are each antibody heavy chain variable domains; 3) (A) and (B) are each antibody light chain variable domains; 4) (A) is an antibody light chain variable domain and (B) is an antibody heavy chain variable domain; or 5) (A) is a VHH and (B) is an antibody light chain variable domain. Additionally or alternatively, preferred embodiments of this aspect include the proviso that when (A) is a V_{H} and (B) is a V_{L}, L1 does not consist of one to five or one to six contiguous amino acids from the amino-terminus of CH1.

In some embodiments, (A) is an antibody variable domain comprising FR1, CDR1, FR2, CDR3, FR3 and CDR3 of a antibody light chain variable domain and FR4 comprising the amino acid sequence GlyGlnGlyThrLysValThrValSerSer (SEQ ID NO:472); and L1 comprises the first 3 to about 12 amino acids of CH1. In particular embodiments, L1 is AlaSerThr (SEQ ID NO:473), AlaSerThrLysGlyProSer (SEQ ID NO:474), or AlaSerThrLysGlyProSerGly (SEQ ID NO:475).

In other embodiments, (A) is an antibody variable domain comprising FR1, CDR1, FR2, CDR3, FR3 and CDR3 of a V_{H} or Vκ domain and FR4 comprising the amino acid sequence GlyXaaGlyThr(Lys/Gln/Glu)(Val/Leu)(Thr/Ile)ValLeu (SEQ ID NO:476); and L1 comprises the first 3 to about 12 amino acids of Cλ.

In other embodiments, (A) is an antibody variable domain comprising FR1, CDR1, FR2, CDR3, FR3 and CDR3 of a V_{H} or Vλ domain and FR4 comprising the amino acid sequence GlyGlnGlyThrLysValGluIleLysArg (SEQ ID NO:477); and L1 comprises the first 3 to about 12 amino acids of Cκ.

In some embodiments, (A) is an immunoglobulin constant domain, such as an antibody constant domain or a TCR constant domain. In particular embodiments, (A) is an antibody heavy chain constant domain, such as CH1, hinge, CH2, or CH3. In some embodiments (A) is a non-human antibody heavy chain constant domain, such as an antibody constant domain from mouse, chicken, pig, torafugu, frog, cow (e.g., Bos taurus), rat, shark (e.g., bull shark, sandbar shark, nurse shark, homed shark, spotted wobbegong shark), skate (e.g., clearnose skate, little skate), fish (e.g., atlantic salmon, channel catfish, lady fish, spotted ratfish, atlantic cod, chinese perch, rainbow trout, spotted wolf fish, zebrafish), possum, sheep, Camelid (e.g., llama, guanaco, alpaca, vicunas, dromedary camel, bactrian camel), rabbit, non-human primate (e.g., new world monkey, old world monkey, cynomolgus monkey (Macaca fascicularis), Callithricidae (e.g., marmosets)), or any other desired non-human species. In more particular embodiments, (A) is a non-human constant domain and (B) is derived from a human polypeptide.

In particular embodiments, (B) is derived from the second polypeptide, wherein the second polypeptide is selected from, for example, a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing. For example, in some fusion proteins (A) is an immuno globulin variable domain (e.g. antibody variable domain), L1 is 1 to about 50 contiguous amino acids that are adjacent to the carboxy-terminus of (A) in a naturally occurring polypeptide that comprises the variable domain A, and (B) is derived from the second polypeptide, wherein the second polypeptide is selected from, for example, a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing.

In other fusion proteins, (A) is derived from the first polypeptide, wherein the first polypeptide is selected from, for example, a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing, L1 is 1 to about 50 contiguous amino acids that are adjacent to the carboxy-terminus of (A) in a naturally occurring polypeptide that comprises (A), and B is an immunoglobulin constant domain. Alternatively, L1 is 1 to about 50 contiguous amino acids that are adjacent to the amino-terminus of (B) in a naturally occurring polypeptide that comprises (B), and (B) is an immunoglobulin constant domain. If desired, the recombinant fusion protein can comprise one or more additional immunoglobulin constant domain that are carboxyl to (B). For example, the fusion protein can comprise an antibody Fc (e.g., optional hinge-CH2-CH3). In further examples, the fusion protein has the structure (A)-L1-CH1-hinge-CH2-CH3; (A)-L1-hinge-CH2-CH3; (A)-L1-CH2-CH3; or (A)-L1-CH3. The constant domains are preferably IgG constant domains, such as IgG1 or IgG4 constant domains.

In particular embodiments, the recombinant fusion protein comprises a first portion derived from an immunoglobulin and a second portion, wherein said first portion is bonded to said second portion through a linker, and the recombinant fusion protein has the formula

(A′)-L2-(B)

wherein (A′) is an immunoglobulin variable domain and (A′) comprises framework (FR) 4 of said immunoglobulin variable domain; L2 is said linker, wherein L2 comprises one to about 50 contiguous amino acids that are adjacent to the carboxy-terminus of said FR4 in a naturally occurring immunoglobulin that comprises said FR4; and (B) is said second portion.

In preferred embodiments, this aspect includes the proviso that (A′) is an antibody variable domain, and L2-B is not a C_{L} or CH1 domain that is peptide bonded to the FR4 of the variable domain (A′) in a naturally occurring antibody that contains the FR4, and when (A′) and (B) are both antibody variable domains 1) (A′) and (B) are each human antibody variable domains; 2) (A′) and (B) are each antibody heavy chain variable domains; 3) (A1) and (B) are each antibody light chain variable domains; 4) (A′) is an antibody light chain variable domain and (B) is an antibody heavy chain variable domain (e.g, VH, VHH); or 5) (A′) is a VHH and (B) is an antibody light chain variable domain. Additionally or alternatively, preferred embodiments of this aspect include the proviso that when (A′) is a V_{H} and (B) is a V_{L}, L2 does not consist of one to five or one to six contiguous amino acids from the amino-terminus of CH1. Additionally or alternatively, preferred embodiments of this aspect include the proviso that (B) is a domain but is not an antibody variable domain. Additionally or alternatively, preferred embodiments of this aspect include the proviso that (B) is, or is derived from, a polypeptide selected from, for example, a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2. EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, or a functional portion of any one of the foregoing. Additionally or alternatively, when (A) and (B) are both antibody variable domains the following is excluded from the invention, (A)-L2-(B) where (A) is a mouse VH, (B) is a mouse VL and L2 is SerAlaLysThrThrPro (SEQ ID NO:537), SerAlaLysThrThrProLysLeuGlyGly (SEQ ID NO:538), AlaLysThrThrProLysLeuGluGluGlyGluPheSerGluAlaArgVal (SEQ ID NO:539), or AlaLysThrThrProLysLeuGluGlu (SEQ ID NO:540). Additionally or alternatively, (A)-L2-(B) is not a fusion protein wherein (A) is a mouse VH, (B) is a mouse VL and L1 is a linker as disclosed in Le Gall et al., Protein Engineering, Design & Selection, 17:357-366 (2004), Kipriyanov et al., Int. J. Cancer, 77:763-772 (1998); Le Gall et al., J. Immunol. Methods, 285:111-127 (2004); Le Gall et al., FEBS Letters, 453:164-168 (1995); or Kipriyanov et al., Protein Engineering, 10:445-453 (1997).

In some embodiments, (A′) is an antibody heavy chain variable domain or a hybrid antibody variable domain, for example, an antibody heavy chain variable domain or a hybrid antibody variable domain that comprises a FR4 that comprises the amino acid sequence GlyXaaGlyThr(Leu/Met/Thr)ValThrValSerSer (SEQ ID NO:478). In particular embodiments, the FR4 comprises GlyXaaGlyThrLeuValThrValSerSer (SEQ ID NO:479), GlyXaaGlyThrMetValThrValSerSer (SEQ ID NO:480), or GlyXaaGlyThrThrValThrValSerSer (SEQ ID NO:481). In such embodiments, L2 comprises one to about 50 contiguous amino acids from the amino-terminus of CH1. For example, L2 can comprise AlaSerThr (SEQ ID NO:473), AlaSerThrLysGlyProSer (SEQ ID NO:474), or AlaSerThrLysGlyProSerGly (SEQ ID NO:475).

In other embodiments, (A′) is a hybrid antibody heavy chain variable domain or a Vk that comprises a FR4 that comprises the amino acid sequence GlyXaaGlyThr(Lys/Arg)(Val/Leu)(Glu/Asp)IleLysArg (SEQ ID NO:485). For example, FR4 can comprise GlyXaaGlyThrLysValGluIleLysArg (SEQ ID NO:486), GlyXaaGlyThrLysLeuGluIleLysArg (SEQ ID NO:487), GlyXaaGlyThrLysValAspIleLysArg (SEQ ID NO:488), or GlyXaaGlyThrArgLysGluIleLysArg (SEQ ID NO:489). In such embodiments, L2 comprises one to about 50 contiguous amino acids from the amino-terminus of Cκ. For example, L2 can comprise ThrValAla (SEQ ID NO:467), ThrValAlaAlaProSer (SEQ ID NO:490), or ThrValAlaAlaProSerGly (SEQ ID NO:491).

In other embodiments, (A′) is a hybrid antibody variable domain or a Vλ that comprises a FR4 that comprises the amino acid sequence GlyXaaGlyThr(Lys/Gln/Glu)(Val/Leu)(Thr/Ile)(Val/Ile)Leu (SEQ ID NO:492). For example FR4 can comprise GlyXaaGlyThrLysValThrValLeu(SEQ ID NO:493), GlyXaaGlyThrLysValThrIleLeu(SEQ ID NO:494), GlyXaaGlyThrLysValIleValLeu(SEQ ID NO:495), GlyXaaGlyThrLysValIleIleLeu(SEQ ID NO:496), GlyXaaGlyThrLysLeuThrValLeu(SEQ ID NO:497), GlyXaaGlyThrLysLeuThrIleLeu(SEQ ID NO:498), GlyXaaGlyThrLysLeuIleValLeu(SEQ ID NO:499), GlyXaaGlyThrLysLeuIleIleLeu(SEQ ID NO:500), GlyXaaGlyThrGlnValThrValLeu(SEQ ID NO:501), GlyXaaGlyThrGlnValThrIleLeu(SEQ ID NO:502), GlyXaaGlyThrGlnValIleValLeu(SEQ ID NO:503), GlyXaaGlyThrGlnValIleIleLeu(SEQ ID NO:504), GlyXaaGlyThrGlnLeuThrValLeu(SEQ ID NO:505), GlyXaaGlyThrGlnLeuThrIleLeu(SEQ ID NO:506), GlyXaaGlyThrGlnLeuIleValLeu(SEQ ID NO:507), GlyXaaGlyThrGlnLeuIleIleLeu(SEQ ID NO:508), GlyXaaGlyThrGluValThrValLeu(SEQ ID NO:509), GlyXaaGlyThrGluValThrIleLeu(SEQ ID NO: 510), GlyXaaGlyThrGluValIleValLeu(SEQ ID NO:511), GlyXaaGlyThrGluValIleIleLeu(SEQ ID NO:512), GlyXaaGlyThrGluLeuThrValLeu(SEQ ID NO:513), GlyXaaGlyThrGluLeuThrIleLeu(SEQ ID NO:514), GlyXaaGlyThrGluLeuIleValLeu(SEQ ID NO:515), and GlyXaaGlyThrGluLeuIleIleLeu(SEQ ID NO:516). Preferably, FR4 comprises GlyXaaGlyThrLysValThrValLeu(SEQ ID NO:493), GlyXaaGlyThrLysLeuThrValLeu(SEQ ID NO:497), GlyXaaGlyThrGlnLeuIleIleLeu(SEQ ID NO:508), GlyXaaGlyThrGluLeuThrValLeu(SEQ ID NO:513), or GlyXaaGlyThrGlnLeuThrValLeu(SEQ ID NO:505). In such embodiments, L2 comprises one to about 50 contiguous amino acids from the amino-terminus of Cλ.

In some embodiments, (B) comprises an immunoglobulin variable domain. Preferably, the immunoglobulin variable domain (e.g., antibody variable domain) is at the amino terminus of (B) and is directly bonded to the carboxy-terminus of L2. In particular examples, the immunoglobulin variable domain is an antibody light chain variable domain or an antibody heavy chain variable domain (e.g., V_{H}, V_{HH}).

In some embodiments, (B) comprises at least a portion of an immunoglobulin constant region. Preferably, said at least a portion immunoglobulin constant region is at the amino terminus of (B) and is directly bonded to the carboxy-terminus of L2. In particular examples, (B) comprises at least a portion of an IgG constant region, such as an IgG1 constant region, an IgG2 constant region, an IgG3 constant region, or an IgG4 constant region. For example, (B) can comprise at least a portion of CH1, at least a portion of hinge, at least a portion of CH2 or at least a portion of CH3. In particular embodiments, (B) comprises at least a portion of hinge, such as a portion of hinge that comprises ThrHisThrCysProProCysPro (SEQ ID NO:520). In other embodiments, (B) comprises at least a portion of hinge and further comprises CH2-CH3. In other embodiments, (′) comprises a portion of CH1-hinge-CH2-CH3, hinge-CH2-CH3, CH2-CH3, or CH3.

In another aspect, the recombinant fusion protein comprises a first portion derived from a first polypeptide and a second portion derived from an immunoglobulin constant region, wherein said first portion is bonded to said second portion through a linker, and the recombinant fusion protein has the formula

(A)-L3-(C³)

wherein (A) is said first portion; (C³) is said second portion derived from an immunoglobulin constant region; and L3 is said linker, wherein L3 comprises one to about 50 contiguous amino acids that are adjacent to the amino-terminus of (C³) in a naturally occurring immunoglobulin that comprises (C³). In certain embodiments of this aspect, the invention includes the proviso that (A) is not a variable domain peptide bonded to L3 in a naturally occurring immunoglobulin comprising L3-(C³).

In preferred embodiments, the first polypeptide is a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing. Thus, in preferred embodiments, (A) is derived from or is a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing.

In some embodiments, (C³) comprises at least one antibody constant domain, such as a human antibody constant domain. Preferably, the antibody constant domain is a human IgG constant domain (e.g., IgG1 constant domain, IgG2 constant domain, IgG3 constant domain, IgG4 constant domain). In some embodiments, (C³) comprises CH3. In these example, 3 can comprise one to about 50 contiguous amino acids from the carboxy-terminus of CH2.

In other embodiments, (C³) comprises CH2 or CH2-CH3, e.g., IgG1 or IgG4 CH2 or CH2-CH3. In these embodiments, L3 can comprise one to about 34 contiguous amino acids from the carboxy-terminus of hinge. For example, L3 can comprise ThrHisThrCysProProCysPro (SEQ ID NO:520) or GlyThrHisThrCysProProCysPro (SEQ ID NO:521). In other embodiments, (C³) comprises hinge. In these embodiments, L3 can comprise one to about 50 contiguous amino acids from the carboxy-terminus of CH1.

In other embodiments, (C³) comprises CH1. In these embodiments, L3 comprises one to about 50 contiguous amino acids from the carboxy-terminus of an antibody heavy chain V domain. For example, L3 can comprise GlyXaaGlyThr(Leu/Met/Thr)ValThrValSerSer (SEQ ID NO:478). In particular embodiments, L3 comprises GlyXaaGlyThrLeuValThrValSerSer (SEQ ID NO:479), GlyXaaGlyThrMetValThrValSerSer (SEQ ID NO:480), or GlyXaaGlyThrThrValThrValSerSer (SEQ ID NO:481).

In some embodiments, (C³) comprises at least a portion of an antibody light chain constant domain. In particular embodiments, (C³) is a Cκ. In such embodiments, L3 comprises one to about 50 contiguous amino acids from the carboxy-terminus of an antibody light chain V domain. For example, L3 can comprise GlyXaaGlyThr(Lys/Arg)(Val/Leu)(Glu/Asp)IleLysArg (SEQ ID NO:485). In particular embodiments, L3 comprises GlyXaaGlyThrLysValGluIleLysArg (SEQ ID NO:486), GlyXaaGlyThrLysLeuGluIleLysArg (SEQ ID NO:487), GlyXaaGlyThrLysValAspIleLysArg (SEQ ID NO:488), or GlyXaaGlyThrArgLysGluIleLysArg (SEQ ID NO:489).

In other embodiments, (C³) is a Cλ. In such embodiments, L3 comprises one to about 50 contiguous amino acids from the carboxy-terminus of an antibody lights chain V domain. For example, L3 can comprise GlyXaaGlyThr(Lys/Gln/Glu)(Val/Leu)(Thr/Ile)(Val/Ile)Leu (SEQ ID NO:492). In particular embodiments, L3 comprises GlyXaaGlyThrLysValThrValLeu(SEQ ID NO:493), GlyXaaGlyThrLysValThrIleLeu(SEQ ID NO:494), GlyXaaGlyThrLysValIleValLeu(SEQ ID NO:495), GlyXaaGlyThrLysValIleIleLeu(SEQ ID NO:496), GlyXaaGlyThrLysLeuThrValLeu(SEQ ID NO:497), GlyXaaGlyThrLysLeuThrIleLeu(SEQ ID NO:498), GlyXaaGlyThrLysLeuIleValLeu(SEQ ID NO:499), GlyXaaGlyThrLysLeuIleIleLeu(SEQ ID NO:500), GlyXaaGlyThrGlnValThrValLeu(SEQ ID NO:501), GlyXaaGlyThrGlnValThrIleLeu(SEQ ID NO:502), GlyXaaGlyThrGlnValIleValLeu(SEQ ID NO:503), GlyXaaGlyThrGlnValIleIleLeu(SEQ ID NO:504), GlyXaaGlyThrGlnLeuThrValLeu(SEQ ID NO:505), GlyXaaGlyThrGlnLeuThrIleLeu(SEQ ID NO:506), GlyXaaGlyThrGlnLeuIleValLeu(SEQ ID NO:507), GlyXaaGlyThrGlnLeuIleIleLeu(SEQ ID NO:508), GlyXaaGlyThrGluValThrValLeu(SEQ ID NO:509), GlyXaaGlyThrGluValThrIleLeu(SEQ ID NO:510), GlyXaaGlyThrGluValIleValLeu(SEQ ID NO:511), GlyXaaGlyThrGluValIleIleLeu(SEQ ID NO:512), GlyXaaGlyThrGluLeuThrValLeu(SEQ ID NO:513), GlyXaaGlyThrGluLeuThrIleLeu(SEQ ID NO:514), GlyXaaGlyThrGluLeuIleValLeu(SEQ ID NO:515), and GlyXaaGlyThrGluLeuIleIleLeu(SEQ ID NO:516). Preferably, L3 comprises GlyXaaGlyThrLysValThrValLeu(SEQ ID NO:493), GlyXaaGlyThrLysLeuThrValLeu(SEQ ID NO:497), GlyXaaGlyThrGlnLeuIleIleLeu(SEQ ID NO:508), GlyXaaGlyThrGluLeuThrValLeu(SEQ ID NO:513), or GlyXaaGlyThrGlnLeuThrValLeu(SEQ ID NO:505).

### Methods for Producing Fusion Proteins

The invention relates to methods for producing fusion proteins that contain one or more natural junctions. The method generally comprises identifying a conserved amino acid sequence motif that is present in two polypeptides or portions thereof that are to be fused. A fusion protein is then prepared that contains the conserved amino acid motif, and in which the amino acid sequence that is adjacent to the amino-terminus of the conserved motif is the same as the amino sequence that is adjacent to the amino-terminus of the conserved motif in one of the original polypeptides, and the amino acid sequence that is adjacent to the carboxy-terminus of the conserved motif is the same as the amino acid sequence that is adjacent to the carboxy-terminus of the conserved motif in the other original polypeptide. Generally, the amino acid sequences of two polypeptides or portions of polypeptides are analyzed to identify a conserved amino acid sequence motif that is present in both of the polypeptides of portions. The analysis can be performed using any suitable method. In one example, the amino acid sequences of a first polypeptide and of a second polypeptide are provided (e.g., from a database) and a conserved amino acid sequence motif present in each polypeptide is identified (e.g., manually or using a suitable sequence analysis software package).

The invention provides a method for producing a fusion protein that comprises at least two portions derived from two different polypeptides, and at least one natural junction between the two portions. If desired, the fusion protein can contain three or more portions, and some of the junctions between portions can be non-natural.

In a general aspect, the invention provides a method of producing a fusion protein comprising a first portion and a second portion that are fused at a natural junction, wherein said first portion is derived from a first polypeptide and said second portion is derived from a second polypeptide. The method comprise analyzing the amino acid sequence of a first polypeptide or a portion thereof and the amino acid sequence of a second polypeptide or a portion thereof to identify a conserved amino acid motif present in the analyzed sequences (the first polypeptide or portion thereof and the second polypeptide or portion thereof); and preparing a fusion protein which has the formula

A-Y-B;

wherein, A is said first portion; Y is said conserved amino acid motif; B is said second portion; and wherein said first polypeptide comprises A-Y, and said second polypeptide comprises Y-B.

The invention also relates to an improved method for making a fusion protein, such as a fusion protein described herein. For example, in some embodiments, the invention relates to an improved method of producing a fusion protein comprising a first portion and a second portion that linked by at least one natural junction, wherein said first portion is derived from a first polypeptide and said second portion is derived from a second polypeptide, the improvement comprising, analyzing the amino acid sequence of said first polypeptide or a portion thereof and the amino acid sequence of said second polypeptide or a portion thereof to identify a conserved amino acid motif present in both of the analyzed sequences; and preparing a fusion protein which has the formula

A-Y-B;

wherein, A is said first portion, Y is said conserved amino acid motif; B is said second portion; and wherein said first polypeptide comprises A-Y, and said second polypeptide comprises Y-B.

The conserved amino acid motif Y can consist of one to about 50 amino acid residues. In certain embodiments, Y consists of about 3 to about 50 amino acids, about 3 to about 40 amino acids, about 3 to about 30 amino acids, about 3 to about 20 amino acids, about 3 to about 15 amino acids, about 3 to about 14 amino acids, about 3 to about 13 amino acids, about 3 to about 12 amino acids, about 3 to about 11 amino acids, about 3 to about 10 amino acids, about 3 to about 9 amino acids, about 3 to about 8 amino acids, about 3 to about 7 amino acids, about 3 to about 6 amino acids, about 3 to about 5 amino acids, at least 8 amino acids, up to about 11 amino acids, or about 8 to about 11 amino acids. In other embodiments, Y consists of about 15 amino acids, about 14 amino acids, about 13 amino acids, about 12 amino acids, about 11 amino acids, about 10 amino acids, about 9 amino acids, about 8 amino acids, about 7 amino acids, about 6 amino acids, about 5 amino acids, about 4 amino acids, about 3 amino acids, about 2 amino acids, or about 1 amino acid.

The conserved amino acid motif Y is found in the first and second polypeptides (parental polypeptides) of which at least a portion is incorporated into a fusion protein of the invention. The fusion protein of the invention, and the hybrid domain in the fusion protein, can contain portions from any desired parental polypeptides provided that each parental protein contains a conserved amino acid motif. For example, the first and second polypeptides (parental polypeptides) can be unrelated (e.g., from different protein superfamilies) or related (e.g., from the same protein superfamily). In certain embodiments, the fusion protein and hybrid domain contains portions derived from first and second polypeptides (parental polypeptides) from the same protein superfamily, such as the immunoglobulin superfamily, the tumor necrosis factor (TNF) superfamily or the TNF receptor superfamily.

The first and second polypeptides (parental polypeptides) can be from the same species or from different species. For example, the first and second polypeptides can independently be from a human (Homo sapiens), or from a non-human species such as mouse, chicken, pig, torafugu, frog, cow (e.g., Bos taurus), rat, shark (e.g., bull shark, sandbar shark, nurse shark, horned shark, spotted wobbegong shark), skate (e.g., clearnose skate, little skate), fish (e.g., atlantic salmon, channel catfish, lady fish, spotted ratfish, atlantic cod, chinese perch, rainbow trout, spotted wolf fish, zebrafish), possum, sheep, Camelid (e.g., llama, guanaco, alpaca, vicunas, dromedary camel, bactrian camel), rabbit, nonhuman primate (e.g., new world monkey, old world monkey, cynomolgus monkey (Macaca fascicularis), Callithricidae (e.g., marmosets)), or any other desired non-human species. In particular embodiments, the first and second polypeptides are both human, or one is human and the other is from a nonhuman species.

The first and second polypeptides (parental polypeptides) can be any desired polypeptides. Suitable examples of first and second polypeptides include a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing.

Conserved amino acid motifs can be readily identified using any suitable method, such as by aligning two or more amino acid sequences and identifying regions of conserved amino acid sequence. This can be accomplished manually or by using any other suitable method, such as using a suitable sequence analysis algorithm or software package (e.g., CLUSTAL (Thompson et al. Nucleic Acids Research, 25:4876-4882 (1997); Chenna R, et al., Nucleic Acids Res, 31:3497-3500. (2003)), BLAST (Altschul, et al., J. Mol. Biol., 215:403-410 (1990), Gish, W. & States, D. J., Nature Genet., 3:266-272 (1993), Madden, et al., Meth. Enzymol., 266:131-141 (1996), Altschul, et al., Nucleic Acids Res., 25:3389-3402 (1997), Zhang et al., J Comput Biol; 7(1-2):203-14 (2000), Zhang, J. & Madden, T. L., Genome Res., 7:649-656 (1997), MOTIF available online from Genomenet, Bioinformatics Center Institute for Chemical Research, Kyoto University (www.genome.jp). For example, as described herein, conserved amino acid motifs that are present in immunoglobulin proteins have been identified by alignment of immunoglobulin amino acid sequences. Particular examples of conserved amino acid motifs include: GlyXaaGlyThr (SEQ ID NO:386) or GlyXaaGlyThrXaa(Val/Leu) (SEQ ID NO:387) in framework region (FR) 4 of antibody variable domains; GluAspThrAla (SEQ ID NO:388), ValTyrTyrCys (SEQ ID NO:389), or GluAspThrAlaValTyrTyrCys (SEQ ID NO:390) in FR3 of antibody variable domains; (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393), or ValThrVal (SEQ ID NO:394) in antibody constant regions.

In some embodiments, the second polypeptide comprises an immunoglobulin constant domain, such as a TCR constant domain or an antibody constant domain. The immunoglobulin constant domain can be a human immunoglobulin constant domain or a nonhuman immunoglobulin constant domain. In one example, the second polypeptide comprises a T cell receptor constant domain.

In certain embodiments, the second polypeptide comprises an antibody light chain constant domain or an antibody heavy chain constant domain, preferably, a human light chain constant domain or a human heavy chain constant domain. In particular embodiments, B comprises an antibody hinge region, a portion of CH1-hinge-CH2-CH3, Fc (hinge-CH2-CH3 or CH2-CH3), or CH3. Preferably, the human antibody heavy chain constant domain is an IgG (IgG1, IgG2, IgG3, IgG4) constant domain. For example, in some embodiments, the IgG constant domain is an IgG1 constant domain or an IgG4 constant domain.

In particular embodiments, the first polypeptide is a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing, and the second polypeptide and B comprise an immunoglobulin constant domain.

In some embodiments, the first polypeptide and A comprise an immunoglobulin variable domain, such as a TCR constant domain or an antibody constant domain. The immunoglobulin variable domain can be a human immunoglobulin variable domain or a nonhuman immunoglobulin variable domain. In one example, the first polypeptide comprises a T cell receptor variable domain.

In certain embodiments, the first polypeptide comprises an antibody light chain variable domain (e.g., Vκ, Vλ) or an antibody heavy chain variable domain (e.g., V_{H}, V_{HH}). In some embodiment, the antibody variable domain is a non-human light chain variable domain or a non-human heavy chain variable domain. For example, the non-human antibody variable domain can be a Camelid antibody variable domain or a nurse shark antibody variable domain. In other embodiments, the antibody variable domain is a human antibody variable domain, such as a human Vk, human Vλ.or human V_{H}.

In particular embodiments, the first polypeptide and A comprise an immunoglobulin variable domain (e.g., antibody variable domain) and said second polypeptide is a cytokine, a cytokine receptor (e.g., an interleukin receptor, such as IL-1R, IL1R Type, a tumor necrosis factor receptor, such as TNFR1, TNFR2), a growth factor (e.g., VEGF, EGF, CSF-1), a growth factor receptor (e.g., VEGF-R1, VEGF-R2, EGFR, CSF-1R), a hormone (e.g., insulin), a hormone receptor (e.g., insulin receptor), an adhesion molecule, a haemostatic factor, a T cell receptor, a T cell receptor chain, a T cell receptor variable domain, an enzyme, a polypeptide comprising or consisting of an antibody variable domain, or a functional portion of any one of the foregoing.

In other embodiments, the first polypeptide is a first antibody chain, and the second polypeptide is a second antibody chain. In such embodiments, Y can be in the variable domain of the first and second antibody chains, or in a constant domain of said first and second antibody chains. For example, Y can be in a framework region of the variable domain of the first and second antibody chains. In a particular embodiment, Y is in FR 4. For example, Y can be GlyXaaGlyThr (SEQ ID NO:386) or GlyXaaGlyThrXaa(Val/Leu) (SEQ ID NO:387). In such embodiments, A comprises a portion of an antibody variable domain comprising FR1, complementarity determining region (CDR) 1, FR2, CDR2, FR3, and CDR3.

In other particular embodiments, Y is in FR3. For example, Y can be GluAspThrAla (SEQ ID NO:388), ValTyrTyrCys (SEQ ID NO:389), or GluAspThrAlaValTyrTyrCys (SEQ ID NO:390). In such embodiments, A comprises a portion of an antibody variable domain comprising FR1, CDR1, FR2, and CDR2.

In other embodiments, Y is in a constant domain (e.g., CH1, hinge, CH2, CH3) of said first antibody chain and a constant domain of said second antibody chain. For example, Y can be (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393) or ValThrVal (SEQ ID NO:394). In particular embodiments, Y is SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), or ValThrVal (SEQ ID NO:394).

When the first polypeptide is a first antibody chain, and the second polypeptide is a second antibody chain, the antibody chains can be from the same or different species. For example, in some embodiments, the first antibody chain and said second antibody chain are both human. In other embodiments, the first antibody chain is human and the second antibody chain is non-human, or the first antibody chain is non-human and the second antibody chain is human.

The recombinant fusion proteins prepared by the methods described herein comprise a partial structure depicted in the formulae presented herein. As described herein, the fusion proteins can comprise additional portions or components that are directly or indirectly fused to the portions specified in the formulae through a natural junction or non-natural junction. For example, if desired the fusion protein of the invention can further comprises a third portion located amino terminally to A. The third portion can be derived from any desired polypeptide. In certain embodiments, the third portion located amino terminally to A is an immunoglobulin variable domain (e.g., antibody variable domain).

The recombinant fusion protein can comprise a hybrid domain, wherein said hybrid domain comprises a first portion derived from a first polypeptide and a second portion derived from a second polypeptide, and a conserved motif that is present in said first polypeptide and in said second polypeptide. This type of recombinant fusion protein can be prepared by a method that comprises analyzing the amino acid sequence of a first domain from a first polypeptide and the amino acid sequence of a second domain from a second polypeptide to identify a conserved amino acid motif present in said first domain and in said second domain, wherein said first domain has the formula (X1-Y-Z1) and said second domain has the formula (X2-Y-Z2), and preparing a fusion protein comprising a hybrid domain that has the formula (X1-Y-Z2), wherein Y is said conserved amino acid motif;

X1 and Z1 are the amino acid motifs that are located adjacent to the amino-terminus of Y in said first polypeptide and said second polypeptide, respectively.

X2 and Z2 are the amino acid motifs that are located adjacent to the carboxy-terminus of Y in said first polypeptide and said second polypeptide, respectively.

In some embodiments, the first polypeptide and the second polypeptide are both members of the same protein superfamily, such as the immunoglobulin superfamily, the TNF superfamily and the TNF receptor superfamily. The first and second polypeptides can both be human polypeptides, or one can be a human polypeptide and the other a non-human polypeptide.

The number of amino acids represented by X1, X2, Z1 and Z2 is dependent on the size of the hybrid domain, and the size of the domains in the parental polypeptides. Generally, X1, X2, Z1 and Z2 each, independently, consist of about 1 to about 400, about 1 to about 200, about 1 to about 100, or about 1 to about 50 amino acids. Similarly, the size of the hybrid domain can vary, and is depend on the size of the domains that contain Y in the parental proteins. In particular embodiments, the hybrid domain is about the size of an immunoglobulin variable domain or immunoglobulin constant domain. In some embodiments, the hybrid domain is about 1 kDa to about 25 kDa, about 5 kDa to about 25 kDa, about 5 kDa to about 20 kDa, about 5 kDa to about 15 kDa, about 6 kDa, about 7 kDa, about 8 kDa, about 9 kDa, about 10 kDa, about 11 kDa, about 12 kDa, about 13 kDa or about 14 kDa.

In some embodiments, the first polypeptide comprises an immunoglobulin variable domain that contains Y, the second polypeptide comprises an immunoglobulin variable domain that contains Y, and (X1-Y-Z2) is a hybrid immunoglobulin variable domain. For example, the first polypeptide can comprises an antibody variable domain, the second polypeptide can comprises an antibody variable domain and Y can be in a framework region (FR), such as FR1, FR2, FR3 or FR 4. In particular examples, Y is in FR4 and is GlyXaaGlyThr (SEQ ID NO:386) or GlyXaaGlyThrXaa(Val/Leu) (SEQ ID NO:387). For example, Y can be GlyXaaGlyThrXaaVal (SEQ ID NO:395) or GlyXaaGlyThrXaaLeu (SEQ ID NO:396). In these embodiments, X1 can be a portion of the antibody variable domain of the first polypeptide that comprises FR1, CDR 1, FR2, CDR2, FR3, and CDR3. In other examples, Y is in FR3 and is GluAspThrAla (SEQ ID NO:388), ValTyrTyrCys (SEQ ID NO:389), or GluAspThrAlaValTyrTyrCys (SEQ ID NO:390). In these embodiments; X1 can be a portion of the antibody variable domain of the first polypeptide that comprises FR1, CDR1, FR2, and CDR2.

In other embodiments, the first polypeptide comprises an immunoglobulin constant domain that contains Y, the second polypeptide comprises an immunoglobulin constant domain, that contains Y and (X1-Y-Z2) is a hybrid immunoglobulin constant domain. For example, Y can be located in an antibody light chain constant domain (e.g., Ck, Cl), or an antibody heavy chain constant domain (e.g., CH1, hinge, CH2, CH3). For example, in an antibody constant domain Y can be (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393) or ValThrVal (SEQ ID NO:394), and in a TCR constant domain Y can be ProSerValPhe (SEQ ID NO:397). In particular embodiments, Y is in an antibody constant domain and is SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), or ValThrVal (SEQ ID NO:394)

In some embodiments, (X1-Y-Z2) is a hybrid immunoglobulin constant domain, and A is an immunoglobulin variable domain. In other embodiments, (X1-Y-Z2) is a hybrid immunoglobulin constant domain, and B is an immunoglobulin constant domain.

In some embodiments the recombinant fusion protein comprises a hybrid immunoglobulin variable domain that is fused to an immunoglobulin constant domain, wherein said hybrid immunoglobulin variable domain comprises a hybrid framework region (FR) that comprises a portion from a first immunoglobulin FR from a first immunoglobulin and a portion from a second immunoglobulin FR from a second immunoglobulin. This type of recombinant fusion protein can be prepared by a method that comprises analyzing the amino acid sequence of a first immunoglobulin FR from a first immunoglobulin and the amino acid sequence of a second immunoglobulin FR from a second immunoglobulin to identify a conserved amino acid motif present in said first immunoglobulin FR and in said second immunoglobulin FR; and preparing a fusion protein comprising a hybrid immunoglobulin FR that has the formula

(F¹-Y-F²),

wherein Y is said conserved amino acid motif; F¹ is the amino acid sequence located adjacent to the amino-terminus of Y in said first immunoglobulin FR; and F² is the amino acid sequence located adjacent to the carboxy-terminus of Y in said second immunoglobulin FR.

The hybrid FR can be a hybrid FR1, hybrid FR2, hybrid FR3 or hybrid FR4. In one example, the first immunoglobulin is an antibody heavy chain, the second immunoglobulin is an antibody light chain, F¹ is derived from FR1, FR2, FR3 or FR4 of the antibody heavy chain variable region, and F² is derived from the corresponding FR of the antibody light chain variable region. In another example, the first immunoglobulin is an antibody light chain, the second immunoglobulin is an antibody heavy chain, F¹ is derived from FR1, FR2, FR3 or FR4 of the antibody light chain variable region, and F² is derived from the corresponding FR of the antibody heavy chain variable region.

In some embodiments, the second immunoglobulin comprises a variable domain containing Y and F² in FR4, and a constant domain. For example, the second polypeptide can be a TCR chain in which Y and F² are in TCR FR4. In this example, the recombinant fusion protein contains a hybrid immunoglobulin domain that is bonded to the amino-terminus of the TCR constant domain. Similarly, the second polypeptide can be an antibody light chain in which Y and F² are in FR4, and the recombinant fusion protein contains a hybrid immunoglobulin domain that is bonded to the amino-terminus of an antibody light chain constant domain. In particular embodiments, the second polypeptide is a κ or λ light chain, F² is derived from a Vκ or Vλ FR4, and the hybrid immunoglobulin domain is bonded to the amino-terminus of Cκ or Cλ, respectively. When the second polypeptide is an antibody heavy chain and F² is derived from an antibody heavy chain variable domain FR4, the hybrid immunoglobulin domain can be bonded to the amino-terminus of an antibody heavy chain constant domain. In particular embodiments, the second polypeptide is an antibody heavy chain, F² is derived from an antibody heavy chain variable domain FR4 (e.g., V_{H} FR4, V_{HH} FR4), and the hybrid immunoglobulin domain is bonded to the amino-terminus of CH1.

In particular embodiments, Y is in FR4 and is GlyXaaGlyThr (SEQ ID NO:386) or GlyXaaGlyThrXaa(Val/Leu) (SEQ ID NO:387). For example, the first immunoglobulin can comprise antibody light chain variable domain comprising an FR4 in which F¹ is Phe and Y is GlyXaaGlyThr (SEQ ID NO:386), and the second immunoglobulin can comprise an antibody heavy chain variable comprising an FR4 domain in which Y is GlyXaaGlyThr (SEQ ID NO:386), and F² is (Leu/Met/Thr)ValThrValSerSer (SEQ ID NO:420). In particular embodiments, F² can be LeuValThrValSerSer (SEQ ID NO:421), MetValThrValSerSer (SEQ ID NO:422), or ThrValThrValSerSer (SEQ ID NO:423).

In other examples, the first immunoglobulin comprises antibody light chain variable domain comprising an FR4 in which F¹ is Phe and Y is GlyXaaGlyThrXaa(Val/Leu) (SEQ ID NO:387), and the second immunoglobulin comprises an antibody heavy chain variable domain comprising an FR4 in which Y is GlyXaaGlyThrXaa(Val/Leu) (SEQ ID NO:387), and F² is ThrValSerSer (SEQ ID NO:419). In particular embodiments, Y is GlyXaaGlyThrXaaVal (SEQ ID NO:395) or GlyXaaGlyThrXaaLeu (SEQ ID NO:396). Preferably the carboxy-terminus of these types of hybrid antibody variable domains is bonded directly to an antibody heavy chain constant domain, such as an IgG (e.g., IgG1, IgG2, IgG3, IgG4) constant domain. Preferably, the antibody heavy chain constant domain is a human antibody heavy chain constant domain. In particular embodiments, the carboxy-terminus of the hybrid antibody variable domain is bonded directly to IgG CH1 or IgG CH2 (e.g., IgG1 CH1, IgG4 CH1, IgG1 CH2, IgG4 CH2).

In other embodiments, the first immunoglobulin comprises antibody heavy chain variable domain comprising an FR4 in which X is Trp, Y is GlyXaaGlyThr (SEQ ID NO:386), and the second immunoglobulin comprises an antibody light chain variable domain comprising an FR4 in which Y is GlyXaaGlyThr (SEQ ID NO:386) and F² is (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425). In particular embodiments, F² is LysValGluIleLys (SEQ ID NO:426), LysValAspIleLys (SEQ ID NO:427), LysLeuGluIleLys (SEQ ID NO:428), LysLeuAspIleLys (SEQ ID NO:429), ArgValGluIleLys (SEQ ID NO:430), ArgValAspIleLys (SEQ ID NO:431), ArgLeuGluIleLys (SEQ ID NO:432), ArgLeuAspIleLys (SEQ ID NO:433), LysValThrValLeu (SEQ ID NO:434), LysValThrIleLeu (SEQ ID NO:435), LysValIleValLeu (SEQ ID NO:436), LysValIleIleLeu (SEQ ID NO:437), LysLeuThrValLeu (SEQ ID NO:438), LysLeuThrIleLeu (SEQ ID NO:439), LysLeuIleValLeu (SEQ ID NO:440), LysLeuIleIleLeu (SEQ ID NO:441), GlnValThrValLeu (SEQ ID NO:442), GlnValThrIleLeu (SEQ ID NO:443), GlnValIleValLeu (SEQ ID NO:444), GlnValIleIleLeu (SEQ ID NO:445), GlnLeuThrValLeu (SEQ ID NO:446), GlnLeuThrIleLeu (SEQ ID NO:447), GlnLeuIleValLeu (SEQ ID NO:448), GlnLeuIleIleLeu (SEQ ID NO:449), GluValThrValLeu (SEQ ID NO:450), GluValThrIleLeu (SEQ ID NO:451), GluValIleValLeu (SEQ ID NO:452), GluValIleIleLeu (SEQ ID NO:453), GluLeuThrValLeu (SEQ ID NO:454), GluLeuThrIleLeu (SEQ ID NO:455), GluLeuIleValLeu (SEQ ID NO:456), or GluLeuIleIleLeu (SEQ ID NO:457).

In other examples, the first immunoglobulin comprises antibody heavy chain variable domain comprising a FR4 in which F¹ is Trp and Y is GlyXaaGlyThrXaaVal (SEQ ID NO:395), and the second immunoglobulin comprises an antibody light chain variable domain comprising an FR4 in which Y is GlyXaaGlyThrXaaVal (SEQ ID NO:395) and F² is (Glu/Asp)IleLys (SEQ ID NO:458) or (Thr/Ile)(Val/Ile)Leu (SEQ ID NO:459). In particular embodiments, F² is GluIleLys (SEQ ID NO:460), AspIleLys (SEQ ID NO:461), ThrValLeu (SEQ ID NO:462), ThrIleLeu (SEQ ID NO:463), IleValLeu (SEQ ID NO:464), or IleIleLeu (SEQ ID NO:465). Preferably the carboxy-terminus of these types of hybrid antibody variable domains is bonded directly to an antibody light chain constant domain, such as Cκor Cλ. Preferably, the antibody light chain constant domain is a human antibody light chain constant domain.

In certain embodiments, the fusion protein produced by this method comprises a hybrid immunoglobulin variable domain that is fused to an immunoglobulin constant domain comprises a partial structure that has the formula (F¹-Y-F 2)-Cκ, (F¹-Y-F²)-Cλ, (F¹-Y-F²)-CH1, (F¹-Y-F²)-CH2 or (F¹-Y-F²)-Fc. In certain embodiments, the fusion protein produced by this method comprises a hybrid immunoglobulin variable domain that is fused to an immunoglobulin constant domain further comprises a second immunoglobulin variable domain (e.g., antibody variable domain). Preferably, the second immunoglobulin variable domain is amino-terminal to the hybrid immunoglobulin variable domain in the fusion protein.

In particular embodiments, the recombinant fusion protein comprises a non-human antibody variable region directly fused to a human antibody constant domain, wherein the non-human antibody variable region comprises a hybrid FR4 having the formula

(F¹-Y-F²)

wherein F¹ is Phe or Trp;

Y is GlyXaaGlyThr (SEQ ID NO:386), and F² is (Leu/Met/Thr)ValThrValSerSer (SEQ ID NO:420), (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425); or

Y is GlyXaaGlyThrXaa(Val/Leu) (SEQ ID NO:387), and F² is ThrValSerSer (SEQ ID NO:419), (Glu/Asp)IleLys (SEQ ID NO:458) or (Thr/Ile)(Val/Ile)Leu (SEQ ID NO:459).

This type of recombinant fusion protein can be prepared by a method that comprises analyzing the amino acid sequence of a first polypeptide that comprises a non-human antibody variable region and the amino acid sequence of and a second polypeptide comprising a human antibody variable domain to identify a conserved amino acid motif Y in FR4 of said non-human antibody variable domain and in FR4 of said human antibody variable domain, and preparing a fusion protein comprising a hybrid FR4 having the formula

(F¹-Y-F²)

wherein F¹ is Phe or Trp;

Y is GlyXaaGlyThr (SEQ ID NO:386), and F² is (Leu/Met/Thr)ValThrValSerSer (SEQ ID NO:420), (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425); or

Y is GlyXaaGlyThrXaa(Val/Leu) (SEQ ID NO:387), and F² is ThrValSerSer (SEQ ID NO:419), (Glu/Asp)IleLys (SEQ ID NO:458) or (Thr/Ile)(Val/Ile)Leu (SEQ ID NO:459).

The non-human antibody variable region can be from any desired species, such as mouse, chicken, pig, torafugu, frog, cow (e.g., Bos taurus), rat, shark (e.g., bull shark, sandbar shark, nurse shark, horned shark, spotted wobbegong shark), skate (e.g., clearnose skate, little skate), fish (e.g., atlantic salmon, channel catfish, lady fish, spotted ratfish, atlantic cod, chinese perch, rainbow trout, spotted wolf fish, zebrafish), possum, sheep, Camelid (e.g., llama, guanaco, alpaca, vicunas, dromedary camel, bactrian camel), rabbit, non-human primate (e.g., new world monkey, old world monkey, cynomolgus monkey (Macaca fascicularis), Callithricidae (e.g., marmosets)), or any other desired non-human species. In certain embodiments, the non-human variable region is a mouse variable region, Camelid variable region, or nurse shark variable region) The second polypeptide can comprise a human heavy chain or light chain variable domain.

In particular examples, the non-human antibody variable domain is a light chain variable domain or a heavy chain variable domain comprising FR4 in which F¹ is Phe or Trp and Y is GlyXaaGlyThr (SEQ ID NO:368), and the second polypeptide comprises a human antibody light chain variable domain comprising FR4 in which F² is (Lys/Arg)(Val/Leu)(Glu/Asp)IleLys (SEQ ID NO:424) or (Lys/Gln/Glu)(Val/Leu)(Thr/Ile)(Val/Ile)Leu (SEQ ID NO:425). Preferably the carboxy-terminus of this type of non-human variable domains that contain a hybrid FR4 is bonded directly to a human antibody light chain constant domain, such as Cκ or Cλ. In other examples, the non-human antibody variable domain is a light chain variable domain or a heavy chain variable domain comprising FR4 in which F¹ is Phe or Trp and Y is GlyXaaGlyThr (SEQ ID NO:386), and the second polypeptide comprises a human antibody light chain variable domain comprising FR4 in which F² is (Leu/Met/Thr)ValThrValSerSer (SEQ ID NO:420). Preferably the carboxy-terminus of this type of non-human variable domains that contain a hybrid FR4 is bonded directly to a human antibody heavy chain constant domain. Preferably, the antibody heavy chain constant domain is a human antibody heavy chain constant domain, such as an IgG (e.g., IgG1, IgG2, IgG3, IgG4) constant domain. In particular embodiments, the human antibody heavy chain constant domain is IgG CH1 or IgG CH2 (e.g., IgG1 CH1, IgG4 CH1, IgG1 CH2, IgG4 CH2).

In particular examples, the non-human antibody variable domain is a light chain variable domain or a heavy chain variable domain comprising FR4 in which F¹ is Phe or Trp and Y is GlyXaaGlyThrXaa(Val/Leu) (SEQ ID NO:387), and the second polypeptide comprises a human antibody light chain variable domain comprising FR4 in which F² is ((Glu/Asp)IleLys (SEQ ID NO:458) or (Thr/Ile)(Val/Ile)Leu (SEQ ID NO:459). Preferably the carboxy-terminus of this type of non-human variable domains that contain a hybrid FR4 is bonded directly to a human antibody light chain constant domain, such as Cκ or Cλ. In other examples, the non-human antibody variable domain is a light chain variable domain or a heavy chain variable domain comprising FR4 in which F¹ is Phe or Trp and Y is GlyXaaGlyThrXaa(Val/Leu) (SEQ ID NO:387), and the second polypeptide comprises a human antibody light chain variable domain comprising FR4 in which F² is ThrValSerSer (SEQ ID NO:419). Preferably the carboxy-terminus of this type of non-human variable domains that contain a hybrid FR4 is bonded directly to a human antibody heavy chain constant domain. Preferably, the antibody heavy chain constant domain is a human antibody heavy chain constant domain, such as an IgG (e.g., IgG1, IgG2, IgG3, IgG4) constant domain. In particular embodiments, the human antibody heavy chain constant domain is IgG CH1 or IgG CH2 (e.g., IgG1 CH1, IgG4 CH1, IgG1 CH2, IgG4 CH2).

In certain embodiments, the fusion protein produced by this method comprises a hybrid immunoglobulin variable domain that is fused to an immunoglobulin constant domain comprises a partial structure that has the formula (F¹-Y-F²)-Cκ, (F¹-Y-F²)-Cλ, (F¹-Y-F²)-CH1, (F¹-Y-F²)-CH2 or (F¹-Y-F²)-Fc. In certain embodiments, the fusion protein produced by this method comprises a hybrid immunoglobulin variable domain that is fused to an immunoglobulin constant domain further comprises a second immunoglobulin variable domain (e.g., antibody variable domain). Preferably, the second immunoglobulin variable domain is amino-terminal to the hybrid immunoglobulin variable domain in the fusion protein.

In some embodiments the recombinant fusion protein an immunoglobulin variable domain fused to a hybrid immunoglobulin constant domain, wherein said hybrid immunoglobulin constant domain comprises a portion from a first immunoglobulin constant domain and a portion from a second immunoglobulin constant domain. This type of recombinant fusion protein can be prepared by a method that comprises analyzing the amino acid sequences of a first immunoglobulin constant domain and a second immunoglobulin constant domain to identify a conserved amino acid motif present in said first immunoglobulin constant domain and in said second immunoglobulin constant domain; and preparing a fusion protein comprising a hybrid immunoglobulin constant domain having the formula

C¹-Y-C²

wherein Y is said conserved amino acid motif;

C¹ is the amino acid sequence adjacent to the amino-terminus of Y in said first immunoglobulin constant domain, and C² is the amino acid sequence adjacent to the carboxy-terminus of Y in said second immunoglobulin constant domain. The hybrid immunoglobulin constant domain can comprise portions from any two immunoglobulin constant domains that contain a conserved amino acid motif. In certain embodiments, the hybrid immunoglobulin constant domain is a hybrid antibody constant domain that comprises a portion from a first antibody constant domain and a portion from a second antibody constant domain. For example, the hybrid antibody constant domain can be a hybrid CH1, hybrid hinge, hybrid CH2 or hybrid CH3, wherein portions of the hybrid domain are derived from antibody constant domains from different species (e.g., human and non-human, such as Camelid or nurse shark) or different isotypes (e.g., IgA, IgD, IgM, IgE, IgG (IgG1, IgG2, IgG3, IgG4)). The hybrid immunoglobulin constant domain can also comprise portions from two different constant domains, such as a portion from a CH1 domain and a portion from a CH2 domain, or from constant domains of different isotypes (e.g., IgG1 and IgG4).

In some embodiments, the method comprises analyzing the sequences of a first immunoglobulin constant domain and a second immunoglobulin constant domain that are from different species. For example, the first immunoglobulin domain can be a non-human antibody constant domain (e.g., Camelid or nurse shark constant domain) and the second immunoglobulin constant domain is a human antibody constant domain. In certain embodiments, the first immunoglobulin constant domain is a Camelid antibody constant domain (e.g., Camelid CH1). In such embodiments, a Camelid VHH can be located amino-terminally to the hybrid constant domain in the fusion protein. For example, the carboxy-terminus of the VHH can be bonded to C¹.

In other embodiments, the method comprises analyzing the sequences of a first immunoglobulin constant domain and a second immunoglobulin constant domain or antibody constant domains of different isotypes. Preferably, the second antibody constant domain is an IgG constant domain (IgG1, IgG2, IgG3, IgG4).

In certain embodiments, the fusion protein comprises an antibody variable domain that is directly bonded to C¹. In such embodiments, the first immunoglobulin constant domain can be the antibody constant domain that is bonded to the variable domain in a naturally occurring antibody. Such constant domains correspond to the variable domain. For example, if the variable domain is a Vκ or Vλ, the first immunoglobulin domain can be a corresponding Cκ or Cλ, respectively. Similarly, if the variable domain is an antibody heavy chain variable domain, the first immunoglobulin variable domain can be a corresponding CH1 domain.

In some embodiments, the method comprises analyzing the amino acid sequence of a first immunoglobulin constant domain that is an antibody light chain constant domain, and the amino acid sequence of a second immunoglobulin constant domain that is an antibody heavy chain constant domain, preferably a human antibody heavy chain constant domain. In some embodiments, the human antibody heavy chain constant domain is a CH1, hinge, CH2 or CH3 domain. Preferably, the human antibody heavy chain constant domain is an IgG (e.g., IgG1, IgG2, IgG3, IgG4) constant domain such as an IgG1 CH1, IgG4 CH1, IgG1 hinge, IgG4 hinge, IgG1 CH2, IgG4 CH2, IgG1 CH3, IgG4 CH3.

In other embodiments, the fusion protein comprises an antibody heavy chain variable domain and the method comprises analyzing the amino acid sequence of a first immunoglobulin constant domain that is a CH1 domain. In such embodiments, the second immunoglobulin constant domain can be an antibody CH1 domain from a different isotype or species, or a different antibody constant domain (e.g., CH2). In a particular embodiment, the second immunoglobulin constant domain is an antibody light chain constant domain.

In some embodiments, the method comprises analyzing the amino acid sequences of a first antibody constant domain and a second antibody constant domain that both contain a conserved amino acid motif (Y) selected (Ser/Ala/Gly)Pro(Lys/Asp/Ser)Val (SEQ ID NO:391), (Ser/Ala/Gly)Pro(Lys/Asp/Ser)ValPhe (SEQ ID NO:392), LysValAspLys(Ser/Arg/Thr) (SEQ ID NO:393), or ValThrVal (SEQ ID NO:394). For example, in particular embodiments, Y is SerProLysVal (SEQ ID NO:398), SerProAspVal (SEQ ID NO:399), SerProSerVal (SEQ ID NO:400), AlaProLysVal (SEQ ID NO:401), AlaProAspVal (SEQ ID NO:402), AlaProSerVal (SEQ ID NO:403), GlyProLysVal (SEQ ID NO:404), GlyProAspVal (SEQ ID NO:405), GlyProSerVal (SEQ ID NO:406), SerProLysValPhe (SEQ ID NO:407), SerProAspValPhe (SEQ ID NO:408), SerProSerValPhe (SEQ ID NO:409), AlaProLysValPhe (SEQ ID NO:410), AlaProAspValPhe (SEQ ID NO:411), AlaProSerValPhe (SEQ ID NO:412), GlyProLysValPhe (SEQ ID NO:413), GlyProAspValPhe (SEQ ID NO:414), GlyProSerValPhe (SEQ ID NO:415), LysValAspLysSer (SEQ ID NO:416), LysValAspLysArg (SEQ ID NO:417), LysValAspLysThr (SEQ ID NO:418), or ValThrVal(SEQ ID NO:394). Preferably, the second antibody constant domain is a human antibody constant domain, and C² is derived from said human antibody constant domain. For example, the human antibody constant domain can be a human Cκ, a human Cλor a human heavy chain constant domain, such as a human CH1, a human hinge, a human CH2 or a human CH3. In particular preferred embodiments, the human antibody constant domain is an IgG CH1 (e.g., IgG1 CH1, IgG4 CH1), IgG hinge (e.g., IgG1 hinge, IgG4 hinge), IgG CH2 (e.g., IgG1 CH2, IgG4 CH2), or IgG CH3 (e.g., IgG1 CH3 or IgG4 CH3), and Z′ is derived from said human antibody constant domain.

Some fusion proteins comprise an antibody light chain variable domain, such as a human light chain variable domain, that is fused to a hybrid antibody CH1 domain, wherein C¹ is GlnProLysAla (SEQ ID NO:466) or ThrValAla (SEQ ID NO:467), and Y is (Ala/Gly)ProSerVal (SEQ ID NO:468). In these embodiments, C² is the amino acid sequence that is adjacent to carboxy-terminus of Y in IgG CH1, such as human IgG CH1 (e.g., IgG1 CH1, IgG4 CH1). This type of fusion protein can be prepared using the methods described herein wherein the amino acid sequence of a Cκ or Cλ domain, and the amino acid sequence of a CH1 domain, are provided.

Some fusion protein comprise an antibody light chain variable domain, such as a human light chain variable domain, that is fused to a hybrid antibody CH2 domain, wherein C¹ is GlnProLysAla (SEQ ID NO:466) or ThrValAla (SEQ ID NO:467), and Y is (Ala/Gly)ProSerVal (SEQ ID NO:468). In such fusion proteins, C² is the amino acid sequence that is adjacent to carboxy-terminus of Y in CH2, such as human IgG CH2 (e.g., IgG1 CH2, IgG4 CH2). This type of fusion protein can be prepared using the methods described herein wherein the amino acid sequence of a Cκ or Cλ domain, and the amino acid sequence of a CH2 domain, are provided.

Some fusion protein comprise an antibody heavy chain variable domain, such as a human heavy chain variable domain, that is fused to a hybrid antibody CH2 domain, wherein C¹ is SerThrLys (SEQ ID NO:469), and Y is (Ala/Gly)ProSerValPhe (SEQ ID NO:470). In these embodiments, C² is the amino acid sequence that is adjacent to the carboxy-terminus of Y in IgG CH2, such as human IgG CH2 (e.g., IgG1 CH2, IgG4 CH2). This type of fusion protein can be prepared using the methods described herein wherein the amino acid sequence of a CH1 domain, and the amino acid sequence of a CH2 domain, are provided.

Some fusion protein comprise an antibody light chain variable domain, such as a human λ chain variable domain, that is fused to a hybrid antibody Cκ domain, wherein C¹ is GlnProLysAla (SEQ ID NO:466), and Y is (Ala/Gly)ProSerVal (SEQ ID NO:468). In these embodiments, Z′ is the amino acid sequence that is adjacent to the carboxy-terminus of Y in Cκ, suc C² as human Cκ. This type of fusion protein can be prepared using the methods described herein wherein the amino acid sequence of a Cλ domain, and the amino acid sequence of a Cκ domain, are provided.

Some fusion protein comprise an antibody heavy chain variable domain, such as a human heavy chain variable domain, that is fused to a hybrid antibody Cκ domain, wherein C¹ is SerThrLys (SEQ ID NO:469), and Y is (Ala/Gly)ProSerValPhe (SEQ ID NO:470). In these embodiments, C² is the amino acid sequence that is adjacent to the carboxy-terminus of Y in Cκ, such as human Cκ. This type of fusion protein can be prepared using the methods described herein wherein the amino acid sequence of a CH1 domain, and the amino acid sequence of a Cκ domain, are provided.

Some fusion protein comprise an antibody light chain variable domain, such as a human κ chain variable domain, that is fused to a hybrid antibody Cλ domain, wherein C¹ is ThrValAla (SEQ ID NO:467), and Y is (Ala/Gly)ProSerVal (SEQ ID NO:468). In these embodiments, C² is the amino acid sequence that is adjacent to the carboxy-terminus of Y in Cλ, such as human Cλ. This type of fusion protein can be prepared using the methods described herein wherein the amino acid sequence of a Cκ domain, and the amino acid sequence of a Cλ domain, are provided.

Some fusion protein comprise an antibody heavy chain variable domain, such as a human heavy chain variable domain, that is fused to a hybrid antibody Cλ domain, wherein C¹ is SerThrLys (SEQ ID NO:469), and Y is (Ala/Gly)ProSerVal (SEQ ID NO:468). In these embodiments, C² is the amino acid sequence that is adjacent to the carboxy-terminus of Y in Cλ, such as human Cλ. This type of fusion protein can be prepared using the methods described herein wherein the amino acid sequence of a CH1 domain, and the amino acid sequence of a Cλ domain, are provided.

The fusion proteins of the invention can be produced using any suitable method. For example, expression of a nucleic acid that encodes the fusion protein or by chemical synthesis. For expression, a nucleic acid encoding the fusion protein can be expressed using any suitable method, (e.g., in vitro expression, in vivo expression). For example, a nucleic acid that encodes a fusion protein of the invention can be inserted into a suitable expression vector. The resulting construct is then introduced into a suitable host cell for expression. Upon expression, fusion protein can be isolated or purified from a cell lysate or preferably from the culture media or periplasm using any suitable method. (See e.g., Current Protocols in Molecular Biology (Ausubel, F. M. et al., eds., Vol. 2, Suppl. 26, pp. 16.4.1-16.7.8 (1991)).

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Suitable expression vectors include, for example, pTT (National Research Council Canada), pcDNA3.1 (Invitrogen), pIRES (Clontech), pEAK8 (EdgeBioSystems), pCEP4 (invitrogen). Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding a fusion protein of the invention, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Recombinant host cells that express a fusion protein of the invention and a method of preparing a fusion protein as described herein are provided. The recombinant host cell comprises a recombinant nucleic acid encoding a recombinant fusion protein. Recombinant fusion proteins can be produced by the expression of a recombinant nucleic acid encoding the protein in a suitable host cell, or using other suitable methods. For example, the expression constructs described herein can be introduced into a suitable host cell, and the resulting cell can be maintained (e.g., in culture, in an animal) under conditions suitable for expression of the constructs. Suitable host cells can be prokaryotic, including bacterial cells such as E. coli, B. subtilis and or other suitable bacteria, eucaryotic, such as fungal or yeast cells (e.g., Pichia pastoris, Aspergillus species, Saccharomyces cerevisiae, Schizosaccharomyces pombe, Neurospora crassa), or other lower eucaryotic cells, and cells of higher eucaryotes such as those from insects (e.g., Sf9 insect cells (WO 94/26087 (O'Connor)) or mammals (e.g., COS cells, such as COS-1 (ATCC Accession No. CRL-1650) and COS-7 (ATCC Accession No. CRL-1651), CHO (e.g., ATCC Accession No. CRL-9096), 293 (ATCC Accession No. CRL-1573), HeLa (ATCC Accession No. CCL-2), CV1 (ATCC Accession No. CCL-70), WOP (Dailey et al., J. Virol. 54:739-749 (1985)), 3T3, 293T (Pear et al., Proc. Natl. Acad. Sci. U.S.A., 90:8392-8396 (1993)), 293-6E cells (National Research Council Canada), NSO cells, SP2/0, HuT 78 cells, and the like (see, e.g., Ausubel, F. M. et al., eds. Current Protocols in Molecular Biology, Greene Publishing Associates and John Wiley & Sons Inc., (1993)).

The invention also includes a method of producing a recombinant fusion protein, comprising maintaining a recombinant host cell of the invention under conditions appropriate for expression of a recombinant fusion protein. The method can further comprise the step of isolating or recovering the recombinant fusion protein, if desired. In another embodiment, the components of the recombinant fusion protein are chemically assembled to create a continuous polypeptide chain.

The invention also provides an isolated recombinant nucleic acid encoding the novel fusion proteins described herein, and a recombinant vector (e.g., expression vector) that contain a recombinant nucleic acid encoding the novel fusion proteins described herein. The invention also relates to an isolated host cell (e.g., non-human host cell) that contains such a nucleic acid or recombinant vector.

The invention also relates to a method for producing a recombinant fusion protein of the invention comprising maintaining host cell (e.g., non-human host cell) that contains a recombinant nucleic acid encoding the novel fusion proteins described herein, or a recombinant vector (e.g., expression vector) that contain a recombinant nucleic acid encoding the novel fusion proteins described herein, under conditions suitable for expression, whereby a recombinant fusion protein is produced. In some embodiments, the method further comprises isolating the recombinant fusion protein (e.g., from the host cell, or the culture medium in which the host cell is maintained.)

### Compositions and Therapeutic and Diagnostic Methods

Compositions comprising fusion proteins of the invention including pharmaceutical or physiological compositions (e.g., for human and/or veterinary administration) are provided. Pharmaceutical or physiological compositions comprise one or more fusion protein and a pharmaceutically or physiologically acceptable carrier. Typically, these carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates. Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compositions can comprise a desired amount of fusion protein. For example the compositions can comprise about 5% to about 99% fusion protein by weight. In particular embodiments, the composition can comprise about 10% to about 99%, or about 20% to about 99%, or about 30% to about 99% or about 40% to about 99%, or about 50% to about 99%, or about 60% to about 99%, or about 70% to about 99%, or about 80% to about 99%, or about 90% to about 99%, or about 95% to about 99% fusion protein, by weight. In one example, the composition is freeze dried (lyophilized).

The drug compositions described herein will typically find use in preventing, suppressing or treating disease states, such as inflammatory states, cancer, pain, and the like. The drug compositions (e.g., drug conjugates, noncovalent drug conjugates, drug fusions), described herein can also be administered for diagnostic purposes.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of drug compositions in protecting against or treating the disease are available. Methods for the testing of systemic lupus erythematosus (SLE) in susceptible mice are known in the art (Knight et al. (1978) J. Exp. Med., 147: 1653; Reinersten et al. (1978) New Eng. J. Med., 299: 515). Myasthenia Gravis (MG) is tested in SJL/J female mice by inducing the disease with soluble AchR protein from another species (Lindstrom et al. (1988) Adv. Immunol., 42: 233). Arthritis is induced in a susceptible strain of mice by injection of Type II collagen (Stuart et al. (1984) Ann. Rev. Immunol., 42: 233). A model by which adjuvant arthritis is induced in susceptible rats by injection of mycobacterial heat shock protein has been described (Van Eden et al. (1988) Nature, 331: 171). Effectiveness for treating osteoarthritis can be assessed in a murine model in which arthritis is induced by intra-articular injection of collagenase (Blom, A. B. et al., Osteoarthritis Cartilage 12:627-635 (2004). Thyroiditis is induced in mice by administration of thyroglobulin as described (Maron et al. (1980) J. Exp. Med., 152: 1115). Insulin dependent diabetes mellitus (IDDM) occurs naturally or can be induced in certain strains of mice such as those described by Kanasawa et al. (1984) Diabetologia, 27: 113. EAE in mouse and rat serves as a model for MS in human. In this model, the demyelinating disease is induced by administration of myelin basic protein (see Paterson (1986) Textbook of Immunopathology, Mischer et al., eds., Grune and Stratton, New York, pp. 179-213; McFarlin et al. (1973) Science, 179: 478: and Satoh et al. (1987) J. Immunol., 138: 179).

The drug compositions of the present invention may be used as separately administered compositions or in conjunction with other agents. Pharmaceutical compositions can include “cocktails” of various cytotoxic or other agents in conjunction with the drug composition of the present invention, or combinations of drug compositions (e.g., fusion proteins) according to the present invention comprising different drugs.

The drug compositions can be administered to any individual or subject in accordance with any suitable techniques. A variety of routes of administration are possible including, for example, oral, dietary, topical, transdermal, rectal, parenteral (e.g., intravenous, intraarterial, intramuscular, subcutaneous, intradermal, intraperitoneal, intrathecal, intraarticular injection), and inhalation (e.g., intrabronchial, intranasal or oral inhalation, intranasal drops) routes of administration, depending on the drug composition and disease or condition to be treated. Administration can be local or systemic as indicated. The preferred mode of administration can vary depending upon the fusion protein chosen, and the condition (e.g., disease) being treated. The dosage and frequency of administration will depend on the age, sex and condition of the patient, concurrent administration of other drugs, counter-indications and other parameters to be taken into account by the clinician. A therapeutically effective amount of a drug composition (e.g., fusion protein) is administered. A therapeutically effective amount is an amount sufficient to achieve the desired therapeutic effect, under the conditions of administration.

The term “subject” or “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species.

The drug composition (e.g., fusion protein) can be administered as a neutral compound or as a salt. Salts of compounds (e.g., fusion proteins) containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

The invention also provides a kit for use in administering a drug composition (e.g., fusion protein) to a subject (e.g., patient), comprising a drug composition (e.g., fusion protein), a drug delivery device and, optionally, instructions for use. The drug composition (e.g., fusion protein) can be provided as a formulation, such as a freeze dried formulation. In certain embodiments, the drug delivery device is selected from the group consisting of a syringe, an inhaler, an intranasal or ocular administration device (e.g., a mister, eye or nose dropper), and a needleless injection device.

The drug composition (e.g., fusion protein) of this invention can be lyophilized for storage and reconstituted in a suitable carrier prior to use. Any suitable lyophilization method (e.g., spray drying, cake drying) and/or reconstitution techniques can be employed. It will be appreciated by those skilled in the art that lyophilisation and reconstitution can lead to varying degrees of antibody activity loss (e.g., with conventional immunoglobulins, IgM antibodies tend to have greater activity loss than IgG antibodies) and that use levels may have to be adjusted to compensate. In a particular embodiment, the invention provides a composition comprising a lyophilized (freeze dried) drug composition (e.g., fusion protein) as described herein. Preferably, the lyophilized (freeze dried) drug composition (e.g., fusion protein) loses no more than about 20%, or no more than about 25%, or no more than about 30%, or no more than about 35%, or no more than about 40%, or no more than about 45%, or no more than about 50% of its activity when rehydrated. Activity is the amount of drug composition (e.g., fusion protein) required to produce the effect of the drug composition before it was lyophilized. For example, the amount of fusion protein needed to achieve and maintain a desired serum concentration for a desired period of time. The activity of the drug composition (e.g., fusion protein) can be determined using any suitable method before lyophilization, and the activity can be determined using the same method after rehydration to determine amount of lost activity.

Compositions containing the drug composition (e.g., fusion protein) or a cocktail thereof can be administered for prophylactic and/or therapeutic treatments. In certain therapeutic applications, an amount sufficient to achieve the desired therapeutic or prophylactic effect, under the conditions of administration, such as at least partial inhibition, suppression, modulation, killing, or some other measurable parameter, of a population of selected cells is defined as a “therapeutically-effective amount or dose.” Amounts needed to achieve this dosage will depend upon the severity of the disease and the general state of the patient's own immune system and general health, but generally range from about 0.005 to 10.0 mg of fusion protein per kilogram of body weight, with doses of 0.05 to 2.0 mg/kg/dose being more commonly used. For prophylactic applications, compositions containing the drug composition (e.g., fusion protein) or cocktails thereof may also be administered in similar or slightly lower dosages. A composition containing a drug composition (e.g., fusion protein) according to the present invention may be utilized in prophylactic and therapeutic settings to aid in the alteration, inactivation, killing or removal of a select target cell population in a mammal.

The invention also relates to a drug delivery device comprising the composition (e.g., pharmaceutical composition) or fusion protein of the invention. In some embodiments, the drug delivery device is selected from the group consisting of parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

It is expected that the conservation of structural features and avoidance of exposure of charged residues, that could be achieved by natural junctions in some circumstances, could be demonstrated in a proteolysis assay. The assay can be carried out as follows. A solution of the recombinant protein (1 mg/mL in phosphate buffered saline) is supplemented with 0.04 mg/mL of sequencing grade trypsin (available from Promega) and incubated at 30° C. At intervals, aliquots of the protein solution are withdrawn, mixed with a stop solution (containing SDS loading buffer and protease inhibitors) and snap frozen. Aliquots are withdrawn after times ranging from, for example, 5 minutes to 24 hours. After completion of the time course, the extent of proteolysis is assessed, for example by separation of samples on SDFS-PAGE gels and visualization with a protein stain such as Coomassie Blue. It is expected that fusion proteins with natural junctions would be more resistant to fragmentation that corresponding fusion proteins that contain non-natural junctions.

### EXAMPLES

### Example 1

### General Methods

### Construction of Expression Vectors

IgGs were expressed using a vector based on the Invitrogen pBudCE4.1 backbone. The backbone was modified by deleting a unique NheI restriction site, which was achieved by NheI restriction digestion, fill-in using Klenow enzyme, and self-ligation, using standard protocols. IgG heavy and light chain expression cassettes comprising a Kozak sequence, murine V-J2-C signal peptide cDNA and constant region cDNA were prepared. The heavy chain expression cassette encoding a human IgG heavy chain constant domain was digested using HindIII and BglII restriction enzymes and sub-cloned into the modified vector backbone that was digested using HindIII and BamHI restriction enzymes, thereby deleting an internal BamHI restriction site in the vector backbone. Light chain expression cassettes encoding human kappa or lambda constant region genes were sub-cloned into the vector backbone using NotI and Mlul restriction enzymes.

### Sub-Cloning of Variable Domain Genes

IgG variable domain genes were sub-cloned into the expression vectors described above using standard molecular biology protocols. IgG variable domain genes used for expression as part of the heavy chain were sub-cloned using a BamHI restriction site in the heavy chain signal peptide cDNA and a XhoI restriction site or NheI restriction site in the cDNA encoding the mature heavy chain protein. IgG variable domain genes used for expression as part of the light chain were subcloned in one of two ways. The IgG variable domain genes were either joined to light chain cDNA using PCR overlap extension, and subsequently sub-cloned using a SalI restriction site in the light chain signal peptide cDNA and the MluI restriction site located downstream of the light chain expression cassette, or they were sub-cloned directly using a SalI restriction site in the cDNA encoding the light chain signal peptide and a BsiWI restriction site in cDNA encoding the mature light chain peptide.

### Expression, Purification and Quantification of IgGs

Following DNA sequence verification, vector DNAs were produced using the Qiagen EndoFree Plasmid Mega kit, according to manufacturer's instructions. The vector DNAs were then used to transfect HEK293T cells (ATCC®). For each construct, cells were typically cultured in 5 or 10 cell culture flasks with a 175 cm² surface area (T175, Nunc) until they reached approximately 70%-80% confluency. Cells were then transfected using 34 microgram of DNA per flask, using FuGENEO® 6 Transfection Reagent (lipid-based transfection reagent, Roche), according to manufacturer's instructions. Transfected cells were grown in DMEM with glutamine and high glucose (Invitrogen) supplemented with 1% non-essential amino acids and 4% foetal bovine serum (FBS). The FBS was prepared from Invitrogen ultra-low IgG FBS by removing residual bovine IgG, using PROSEP®-G resin (recombinant protein G resin, Millipore), followed by sterile filtration. Culture supernatants were harvested by centrifugation after 4 or 5 days of expression. Secreted IgG was affinity purified using protein A resin (Streamline A, GE Healthcare) in the case of IgG molecules comprising 2 VH and 2 Vκ domains or 4 Vκ domains, or using protein G resin engineered without Fab binding sites (protein G agarose, Sigma Aldrich) in the case of IgG molecules comprising 4 VH domains. Resins were typically washed using 20-50 bed volumes of 2×PBS followed by 10-20 bed volumes of 150 mM NaCl, 10 mM Tris HCl, pH 7.4. IgGs were typically eluted using either 100 mM glycine pH 2.0 and neutralized to pH 8.0 using Tris, or they were eluted using 10 mM citrate, 50% ethylene glycol, pH 3.5. Eluted proteins were quantified by absorbance reading at 280 nm, using a spectrophotometer.

### Size Exclusion Chromatography

IgGs were analyzed by HPLC size exclusion chromatography, using CHROMELEON® software (chromatography management software, Dionex Corporation). Analysis parameters most typically included using a Tosoh G3000 SWXL column, with 1×PBS supplemented with 10% ethanol as running buffer at a 1 mL/min flow rate, and an acquisition period of 20 minutes following injection. Absorbance was recorded at 225, 280 and 300 nm wavelengths.

### Example 2

### Protein Expression and Formation of Soluble Oligomers and Aggregates

Two Vκ variable domains, designated DOM9-155-25 and DOM10-176-535 were paired into IgGs containing a total of 4 Vκ variable domains per molecule.

The Vκ domain DOM10-176-535 was expressed as part of a native light chain while the Vκ domain DOM9-155-25 was fused to CH1 on the heavy chain, using three different junctions.

| | |
|---|---|
| Kabat number: | 97 114 |
| Unnatural junction 1: | TFGQGTKVEIK ASTKGPS |
| Unnatural junction 2: | TFGQGTKVEIKR ASTKGPS |
| Natural junction: | TFGQGTLVTVSS ASTKGPS |

For each junction the fusion is underlined

Unnatural junction 1 (SEQ ID NO:522) represents the direct fusion of a Vκ domain comprising Kabat residues 1-112 with CH1, while unnatural junction 2 (SEQ ID NO:523) represents the direct fusion of a Vκ domain also comprising Kabat residue 113 (partially encoded by the Jκ exon and partially by the Cκ exon in humans) with CH1. In IgGs with the natural junction (SEQ ID NO:524) the conserved GlyXaaGlyThr motif(SEQ ID NO:386) (residues H104-H107 in VH domains and L99-L102 in Vκ domains) was used as the fusion site.

Expression yields were compared using absorbance reading at 280 nm wavelength and confirmed by size exclusion HPLC and SDS-PAGE. The yields are summarized in Table 1. The expression yield was significantly higher with the natural junction (SEQ ID NO:524) than with either unnatural junction 1 (SEQ ID NO:522) or unnatural junction 2 (SEQ ID NO:523).

The use of natural junctions reduced the proportion of soluble oligomers and aggregates compared to using unnatural junctions for some antibodies. For example, three IgGs were expressed which comprised the same Vκ domain, designated VκDUM-1, as part of a native light chain and fused to CH1 on the heavy chain using different junctions. The three IgGs were analyzed by size exclusion HPLC (Table 2). The fraction of oligomers and aggregates was 9% for the IgG with unnatural junction 1 (SEQ ID NO:522) and 10% for the IgG with unnatural junction 2 (SEQ ID NO:523), but only 7% for the IgG with the natural junction (SEQ ID NO:524), indicating that fewer oligomers and aggregates were expressed and purified when the natural junction was used. A reduction in oligomers and aggregates by a few percent provides advantages and reduces the costs and time required to produce the fusion proteins, especially for industrial scale production.

TABLE 1

| | | | Expression |
|---|---|---|---|
| Variable domain | Variable domain | | yield |
| fused to Cκ | fused to CH1 | Junction | (mg/L) |
| DOM10-176-535 | DOM9-155-25 | Natural junction | 1.4 |
| DOM10-176-535 | DOM9-155-25 | Unnatural junction 1 | 1.0 |
| DOM10-176-535 | DOM9-155-25 | Unnatural junction 2 | 0.4 |

TABLE 2

| | | | Percentage |
|---|---|---|---|
| | | | of oligomers |
| Variable | Variable | | and aggregates |
| domain fused | domain fused | | purified |
| to Cκ | to CH1 | Junction | on protein A |
| VHDUM-1 | VκDUM-1 | Natural junction | 7 |
| VHDUM-1 | VκDUM-1 | Unnatural junction 1 | 9 |
| VHDUM-1 | VκDUM-1 | Unnatural junction 2 | 10 |

### Example 3

### Protein Solubility

A VH variable domain, designated VHDUM-1, was expressed in IgG molecules containing 4 copies of this variable domain. The solubility of three molecules was compared, two of the molecules had an unnatural junction between VHDUM-1 and Cκ and one domain had a natural junction between VHDUM-1 and Cκ.

| | |
|---|---|
| Kabat number: | 100 109 |
| Unnatural junction 1: | FDYWGQGTLVTVSS TVAAPS |
| Unnatural junction 2: | FDYWGQGTLVTVSS RTVAAPS |
| Natural junction: | FDYWGQGTKVEIK R TVAAPS |

For each junction the fusion site is underlined

Following elution from protein G resin and neutralization, strong precipitation was observed for the IgG with unnatural junction 1 (SEQ ID NO:525), while only traces of precipitation were observed for the IgG with unnatural junction 2 (SEQ ID NO:526) and for the IgG with the natural junction (SEQ ID NO:527). The concentration of soluble protein remaining in solution after neutralization (100 mM glycine, 130 mM Tris pH8) was 0.16 mg/mL for the IgG with unnatural junction 1, 1.37 mg/mL for the IgG with unnatural junction 2, and 1.20 mg/mL for the IgG with the natural junction. The data demonstrated that the IgG with unnatural junction 1 had significantly lower solubility in 100 mM glycine, 130 mM Tris pH 8 than the other IgGs. This result suggested that residue L108 (Arg) that is part of the natural junction between Vκ and Cκ domains played an important role in the structure and solubility of the tested IgGs. This residue is partially encoded by the Jκ exon and partially by the Cκ exon in humans and was absent in the poorly soluble IgG with unnatural junction 1. The observed differences in solubility demonstrated the benefit of moving the domain fusion site to the GlyXaaGlyThr (SEQ ID NO:386) motif that is conserved between Vκ (residues L99-L102) and VH (residues H104-H107), thus preserving the remaining structurally important Vκ residues encoded by the Jκ exon downstream of the conserved motif.

### Example 4

### Cloning, Expression and Characterization of DOM15/16 Inline Fusions

Domain antibodies that bind VEGF or EGFR were incorporated into fusion polypeptides that contained an anti-VEGFR dAb and an anti-EGFR dAb in a single polypeptide chain. Some of the fusion polypeptides also included an antibody Fc region (—CH2-CH₃ of human IgG1). Specific examples of the fusion polypeptides that were cloned and expressed include TAR15-10 fused to DOM16-39-206 and to Fc; DOM16-39-206 fused to TAR15-10 and to Fc; DOM16-39-206 fused to TAR15-26-501 and to Fc; TAR15-26-501 fused to DOM16-39-206 and to Fc; TAR15-10 fused to DOM16-39-206; DOM16-39-206 fused to TAR15-10; DOM16-39-206 fused to TAR15-26-501; and TAR15-26-501 fused to DOM16-39-206. The positions of the foregoing fusions are listed as they appear in the fusion proteins from amino terminus to carboxy terminus. Polypeptides that are referred to using the prefix TAR or DOM are antibody variable domains.

DNA encoding dAbs was PCR amplified and cloned into expression vectors using standard methods. Inline fusion polypeptides were produced by expressing the expression vectors in Pichia (fusion that did not contain an Fc region) or in HEK 293T cells (Fc region containing fusions). Inline fusions were batch bound and affinity purified on streamline protein A and streamline protein L resins for HEK 293T cells (Fc-tagged) and Pichia expressed constructs respectively.

The portions of several fusions that contain Fc are listed in Table 3 as they appear in the fusion proteins, from amino terminus to carboxy terminus. Accordingly, the structure of the fusion proteins can be appreciated by reading the table from left to right. The first fusion protein presented in Table 3 has the structure, from amino terminus to carboxy terminus, DOM15-10-Linker 1-DOM16-39-206-Linker 2-Fc.

General robustness and resistance to degradation were tested by subjecting the inline fusions to proteolysis with trypsin. A solution of dual specific ligand and trypsin (1/25 (w/w) trypsin to ligand) was prepared and incubated at 30° C. Samples were taken at 0 minutes (i.e., before addition of trypsin), 60 minutes, 180 minutes, and 24 hours. At the given time points, the reaction was stopped by the addition of complete protease inhibitor cocktail at 2× final concentration (Roche code: 11 836 145 001) with PAGE loading dye, followed by flash freezing the samples in liquid nitrogen. Samples were analyzed by SDS-PAGE, and protein bands were visualized to reveal a time course for the protease degradation of the fusions.

These experiments showed that inline fusions having a “natural” linker (KVEIKRTVAAPS (SEQ ID NO:528), which contains the carboxy-terminal amino acids of Vκ and amino-terminal amino acids of Cκ, were susceptible proteolysis, with degradation evident at the 10 minute time point. SDS-PAGE analysis revealed that degradation occurred at the linkers between dAbs and at the linkers between dAb and Fc.

New linkers were designed that contain fewer Lys and Arg residues, which are cleavage points for trypsin and are abundant in the natural linker. Fusions that contained the engineered linkers (LVTVSSAST (SEQ ID NO:529)) or (LVTVSSGGGGSGGGS (SEQ ID NO:530)) showed much improved resistance to trypsin proteolysis.

Additional binding assays were performed to assess the potency of the inline fusions that contained the engineered linkers. The results revealed engineered linkers did not have any substantial adverse effect on potency.

TABLE 3
Fusion polypeptides that contain Fc

| | | | | | |
|---|---|---|---|---|---|
| | | | | Assay | Assay |
| | | | | dAb1 | dAb2 |
| dAb1 | Linker 1 | dAb2 | Linker 2 | (nM) | (nM) |

| | | | | | |
|---|---|---|---|---|---|
| DOM15- | KVEIKRTVAAPS | DOM16- | KVEIKRTVAAPS | 0.45 | 23.8 |
| 10 (Vκ) | | 39-206 | | | |
| | | (Vκ) | | | |
| DOM16- | KVEIKRTVAAPS | DOM15- | KVEIKRTVAAPS | 3.7 | 0.88 |
| 39-206 | | 10 (Vκ) | | | |
| (Vκ) | | | | | |
| DOM16- | KVEIKRTVAAPS | DOM15- | LVTVSSASTKGPS | 20.7 | 21.3 |
| 39-206 | | 26-501 | | | |
| (Vκ) | | (V_{H}) | | | |
| DOM15- | LVTVSSASTKGPS | DOM16- | KVEIKRTVAAPS | 5.7 | 7.7 |
| 26-501 | | 39-206 | | | |
| (V_{H}) | | (Vκ) | | | |
| DOM16- | LVTVSSAST | DOM15- | LVTVSSAST | 0.68 | 10.8 |
| 39-601 | | 10 (Vκ) | | | |
| (Vκ) | | | | | |
| DOM16- | KVEIKRTVAAPS | DOM15- | KVEIKRTVAAPS | 0.77 | 2.9 |
| 39-601 | | 10 (Vκ) | | | |
| (Vκ) | | | | | |
| DOM15- | LVTVSSAST | DOM16- | LVTVSSAST | 1.2 | 4.2 |
| 10 (Vκ) | | 39-601 | | | |
| | | (Vκ) | | | |
| DOM16- | LVTVSSGGGGSGGGS | DOM15- | LVTVSSGGGGSGGGS | 5.7 | 0.2 |
| 39-601 | | 10 (Vκ) | | | |
| (Vκ) | | | | | |
| DOM15- | LVTVSSGGGGSGGGS | DOM16- | LVTVSSGGGGSGGGS | 0.8 | 3.1 |
| 10 (Vκ) | | 39-601 | | | |
| | | (Vκ) | | | |
| DOM15- | KVEIKRTVAAPS | DOM16- | KVEIKRTVAAPS | 0.2 | 2.9 |
| 10 (Vκ) | | 39-601 | | | |
| | | (Vκ) | | | |

### Example 5

### Additional Engineered Linkers

Several designed mutations were introduced to the C-terminal region of Vκ dAbs expressed on the light chain of IgG-like formats to reduce protease sensitivity. The “natural linker” was GQGTKVEIKRTVAAPS (SEQ ID NO:531) which contains the carboxy-terminal amino acids of Vκ and amino-terminal amino acids of Ck). Variant linkers 1-3 were designed with amino acid replacements that replaced some or all of the positively charged residues in the natural linker with the most conservative substitutions that are not positively charged at physiological pH. It is likely that the arginine residue in the natural linker is less amenable to alteration due to ionic interactions it forms within the CL domain.

Variant linker 1 (GQGTNVEINRTVAAPS (SEQ ID NO:532)) substitutes both lysines in the natural linker with asparagines. Variant linker 1, and variant linker 2 (GQGTNVEINQTVAAPS (SEQ ID NO:533)), which additionally changes the arginine in the natural linker to glutamine, introduce an N-glycosylation site (N×T) into the linker. SDS-PAGE analysis of IgG-like formats containing variant linker 1 or variant linker 2 showed that the light chain had a higher molecular weight, consistent with an N-glycosylation event. Variant linker 3 (GQGTNVEIQRTVAAPS (SEQ ID NO:534) removes the N-glycosylation site while leaving the arginine in the natural linker in place. Variant linker 4 (GQGTLVTVSSTVAAPS (SEQ ID NO:535)) replaces the six C-terminal amino acids of the Vκ domain with the corresponding residues from a VH domain, and is devoid of positive charges.

Protease resistance (trypsin resistance assessed as described in Example 4) of IgG-like formats that contain variant linkers 1-4 revealed that IgG-like formats that contained engineered variant linkers were more protease resistant than an IgG-like format that contained the natural linker.

### Example 6

### Cloning, Expression and Characterization of DOM9/10 Inline Fusions

### A. Fusion Proteins

### Cloning and Production of Anti-IL-4 and Anti-IL-13 Dual Specificity Dimer

Nucleic acids encoding the anti-IL-4 dAb DOM9-112 and anti-IL-13 dAb DOM10-53-343 were cloned into a construct that encoded an in-line fusion protein with a C-terminal cysteine. The amino acid sequence AST was present between the two dAbs, this sequence is the natural CH sequence present in natural antibodies. The construct was cloned in the Pichia pastoris vector pPICZα (Invitrogen). Electrocompetent cells (X-33 or KM71H) were transformed with the construct and transformants were selected on 100 μg/ml Zeocin. 500 ml cultures were grown on BMGY media at 30° C., 250 rpm for 24 hrs until the OD₆₀₀ had reached ˜15-20. The cells were then spun down and resuspended in BMMY media (containing 0.5% (v/v) methanol) to induce protein expression. The cultures were maintained at 30° C. with shaking at 250 rpm. At 24 hour intervals the cultures were fed with the following incremental increase in the methanol concentration; 1%, 1.5% and 2% (v/v) using a 50% methanol solution. The cultures were then harvested by centrifugation and the supernatant containing the expressed protein stored at 4° C. until required. The protein was purified from the supernatant using PrA streamline using the standard purification protocol.

The PrA purified protein was found to contain both dimer and monomer species. Therefore chromatofocusing was used to separate the two proteins. A Mono P 5/20 column was used (GE Healthcare) for the separation, using a pH gradient of 6 to 4. The poly-buffers used were as described by the manufacturer to make the 6 to 4 pH range. The sample was applied at pH6 and the pH gradient generated by using 100% buffer B over 35 column volumes run at 1 ml/min. Dimer containing fractions were identified using SDS-PAGE and pooled for PEGylation.

The protein was then PEGylated using 40K PEG2-MAL using the method outlined above. This material was purified using anion exchange chromatography up to a purity >95%. The potency of the resulting dual specific ligand (PEGylated DOM9-112 (AST) DOM10-53-344) was determined in an IL-4 RBA and an IL-13 RBA. The potency of the anti-IL-4 arm of the dual specific ligand (13 nM) was slightly reduced compared with the potency of the dAb DOM9-112 monomer (3.5 nM), whereas the potency of the anti-IL-13 arm was maintained (310 pM for the dual specific ligand vs 230 pM for the dAb monomer).

The anti-IL-4 and anti-IL-13 dAbs DOM9-112 and DOM10-53-344 were also cloned as an in-line fusion with the amino acid sequence ASTKGPS (SEQ ID NO:535) present between the two dAbs, this sequence is the start of the CH sequence present in natural antibodies. The potency of the resulting purified dual specific ligand (DOM9-112 (ASTKGPS) DOM10-53-344) was determined in an IL-4 RBA and an IL-13 sandwich ELISA. The potency of the anti-IL-4 arm was maintained (˜1 nM) whereas the potency of the anti-IL-13 arm was only slightly reduced compared with the dAb monomer (40 pM for the dAb monomer vs 120 pM for the dual specific ligand).

Additional Dual Targeting in-Line Fusions for IL-4 and IL-13.

To further understand the behaviour of dual targeting in-line fusions of IL4 and IL13 binding dAbs, a series of new in-line fusions and in-line fusion libraries were constructed. The DOM10-53 lineage was affinity matured using phage display using libraries diversifying triplet residues of FR1, CDR1, CDR2 and CDR3. The libraries were cloned in a phage vector and displayed as fusion protein to the gene3 protein as an (dAb1 linker dAb2) in-line fusion with dAb1 being DOM9-112-210, the linker being amino acid residues ASTKGPS (SEQ ID NO:535) and dAb2 being the DOM10-53 library. The selection method, subcloning and expression in E coli and screening method were essentially performed as described above, except that in-line fusion constructs were used instead of single dAbs. Outputs were cloned into vector pDOM5 and expression supernatants were screened for improved expression by binding to a protein A coated Biacore chip.

In-line fusions with improved expression levels were expressed, purified and tested in a IL-13 sandwich ELISA and cell assay. A number of variants were selected (including DOM9-112-210-ASTKGPS-DOM10-53-566). The most potent clones were DOM10-53-531 and DOM10-53-546 (see Table 4). Different protein preparations were made from these clones and these were tested in the IL-4 RBA and IL-13 sandwich assay as described above.

TABLE 4

| | Expression level | IL-13 Sanwich | IL-4 RBA |
|---|---|---|---|
| Clone name | (mg/l) | ELISA (EC50 nM) | (IC50 nM) |
| DOM9-112-210- | | | |
| DOM10-53-531 | | | |
| Prep 1 | 9.3 | 1.1/1.9 | 3.5/4.8 |
| Prep 2 | 11.5 | 4.9 | n.d. |
| Prep 3 | 4.5 | 2/2.8 | 13.9 |
| Prep 4 | 10 | 1 | 5.4 |
| DOM9-112-210- | | | |
| DOM10-53-546 | | | |
| Prep 1 | 2.2 | 0.62/0.77 | 4.3 |
| Prep 2 | 7.7 | 1 | 6 |

Further in-line fusions were constructed by SOE PCR of the DNA fragments encoding a dAb linker which is either ASTKGPS (SEQ ID NO:535), if the first dAb was a Vh, or TVAAPS (SEQ ID NO:536) if the first dAb was a Vκ. This PCR product was digested with SalI/NotI and ligated in the E. coli expression vector pDOM5. After transformation to MACH1 (Invitrogen) cells, the clones were sequence verified and the in-line fusions were expressed. Expression was done by growing E. coli in 2TY supplemented with Onex media (Novagen) for 2 nights at 30° C., the cells were centrifuged and the supernatant was incubated with either Protein-L or Protein-A resin. After elution from the resin, the quality and quantity of produced in-line fusion product was verified on SDS-PAGE. The vast majority of product formed had the molecular mass of an in-line fusion with only limited free monomer. Therefore, no additional purification steps were required and the material could be tested directly.

Using the above described method the following IL-4/IL-13 in-line fusions were expressed, purified and characterised:

DOM9-112-210 - ASTKGPS - DOM10-208
DOM9-112-210 - ASTKGPS - DOM10-212
DOM9-112-210 - ASTKGPS - DOM10-213
DOM9-112-210 - ASTKGPS - DOM10-215
DOM9-112-210 - ASTKGPS - DOM10-224
DOM9-112-210 - ASTKGPS - DOM10-270
DOM9-112-210 - ASTKGPS - DOM10-416
DOM9-112-210 - ASTKGPS - DOM10-236
DOM9-112-210 - ASTKGPS - DOM10-273
DOM9-112-210 - ASTKGPS - DOM10-275
DOM9-112-210 - ASTKGPS - DOM10-276
DOM9-112-210 - ASTKGPS - DOM10-277
DOM10-208 - TVAAPS - DOM9-155-78
DOM10-212 - TVAAPS - DOM9-155-78
DOM10-213 - TVAAPS - DOM9-155-78
DOM10-215 - TVAAPS - DOM9-155-78
DOM10-224 - TVAAPS - DOM9-155-78
DOM10-270 - TVAAPS - DOM9-155-78
DOM10-416 - ASTKGPS - DOM9-155-78
DOM10-236 - ASTKGPS - DOM9-155-78
DOM10-273 - ASTKGPS - DOM9-155-78
DOM10-275 - ASTKGPS - DOM9-155-78
DOM10-276 - ASTKGPS - DOM9-155-78
DOM10-277 - ASTKGPS - DOM9-155-78

Once purified, the expression levels were determined (mg/l) and the activities were tested in an RBA for IL-4 binding and in a sandwich ELISA for IL-13 binding. The amino acid sequences of the listed variable domains are disclosed in the International Patent Application by Domantis Limited, entitled Ligands that Bind IL-4 and/of IL-13, which was filed in the UK receiving office on Jan. 24, 2007, and are incorporated herein by reference for the purpose of providing examples of variable domains that can be used to make fusion proteins that contain natural junctions. The table below (Table 5) summarizes the data for these in-line fusions:

TABLE 5

| | | | | | Dom10 | DOM9 |
|---|---|---|---|---|---|---|
| | Expression | | | IL-13 | RBA | RBA (IC50 |
| clone name | mg/ml | | | Biacore | (EC50 nM) | nM) |
| DOM9-112-210-DOM10-208 | 0.3 | 19.2 | 37.4 | ✓ | — | 2.48 |
| DOM9-112-210-DOM10-212 | 3.7 | | 6.3 | ✓ | 999.9 | 3.21 |
| DOM9-112-210-DOM10-213 | 5.6 | 0.2 | 4.9 | ✓ | 1152 | 4.29 |
| DOM9-112-210-DOM10-215 | 0.1 | 8.8 | 2.2 | ✓ | — | 14.19 |
| DOM9-112-210-DOM10-224 | 4.6 | | 13.4 | ✓ | 4575 | 2.75 |
| DOM9-112-210-DOM10-270 | 3.7 | | 2.7 | ✓ | 397.5 | 2.79 |
| DOM9-112-210-DOM10-416 | 6.9 | | 0.0 | ✓ | 34420 | 7.27 |
| DOM9-112-210-DOM10-236 | 0.2 | 0.1 | 2.2 | ✓ | — | >20 |
| DOM9-112-210-DOM10-273 | 1.2 | | 0.3 | ✓ | 4553 | 10.51 |
| DOM9-112-210-DOM10-275 | 4.9 | 0.2 | 0.0 | ✓ | — | 10.89 |
| DOM9-112-210-DOM10-276 | 6.9 | | 0.1 | ✓ | — | 10.20 |
| DOM9-112-210-DOM10-277 | 1.3 | 3.7 | 0.2 | ✓ | 4385 | 11.74 |
| DOM10-208-DOM9-155-78 | | | 41.0 | ✓ | 4243 | 8.18 |
| DOM10-212-DOM9-155-78 | | | 0.5 | ✓ | — | >20 |
| DOM10-213-DOM9-155-78 | | | 16.9 | ✓ | 62.04 | 6.91 |
| DOM10-215-DOM9-155-78 | | | 22.6 | ✓ | 10.82 | 6.65 |
| DOM10-224-DOM9-155-78 | | | 3.6 | ✓ | — | 12.49 |
| DOM10-270-DOM9-155-78 | | | 2.9 | ✓ | 37.23 | 8.60 |
| DOM10-416-DOM9-155-78 | | 1.1 | 26.3 | ✓ | 443.7 | 5.88 |
| DOM10-236-DOM9-155-78 | | 3.6 | 10.8 | ✓ | 372 | 2.54 |
| DOM10-273-DOM9-155-78 | | 6.4 | 16.2 | ✓ | 185.2 | 2.25 |
| DOM10-275-DOM9-155-78 | | 0.2 | 0.0 | — | — | — |
| DOM10-276-DOM9-155-78 | | 0.2 | 20.0 | ✓ | — | 5.02 |
| DOM10-277-DOM9-155-78 | | 1.1 | 1.3 | ✓ | 648 | 9.45 |
| | | | | | DOM9-112 | 3.60 |
| | | | | | DOM9- | 0.41 |
| | | | | | 155-78 | |

Furthermore, an affinity matured variant of DOM10-275, i.e. DOM10-275-1, was specifically chosen to be paired with both DOM9-112-210 and DOM9-155-78. These in-line fusions were constructed and expressed as described above using a natural linker. In addition to testing in the mentioned IL-4 RBA and IL-13 sandwich ELISA, these in-line fusions were also tested for functionality in a TF-1 cell proliferation assay. In these assays the dAb was preincubated with a fixed amount of either IL-4 or IL-13, this mixture was added to the TF-1 cells and the cells were incubated for 72 hours. After this incubation, the level of cell proliferation was determined. The results of this assay are summarized below (Table 6) and demonstrate that both arms of the in-line fusion were active in the cell assay.

TABLE 6

| | | | Il-4 cell | IL-13 cell |
|---|---|---|---|---|
| | DOM9 RBA | DOM10 RBA | assay | assay |
| Sample | IC50 (nM) | IC50 (nM) | IC50 (nM) | IC50 (nM) |
| DOM9-112-210 | 0.391 | — | | |
| DOM9-155-78 | 0.456 | — | | |
| DOM10-275-1- | 6.238 | 39.17 | 5.1-7.6 | 31-46 |
| DOM9-155-78 | | | | |
| DOM9-112-210- | 4.189 | 44.88 | 6.8-10.2 | 27-40 |
| DOM10-275-1 | | | | |
| DOM10-275-1 | — | 31.30 | | |

TABLE 7
IgGs including 4 VH variable domains expressed with natural junctions

| | | | | |
|---|---|---|---|---|
| | | | Junction | |
| | | | between GQGT | |
| | | | in JH-segment | Non-native |
| | Heavy chain | Light chain variable | and non-native | constant |
| Number | variable domain | domain | constant domain | domain |

| | | | | |
|---|---|---|---|---|
| 1. | VHDUM-1 | VHDUM-1 | KVEIKR (SEQ | CK |
| | | | ID NO: 471) | |
| 2. | VHDUM-1 | VHDUM-1 | KVTVL (SEQ | CL2 |
| | | | ID NO: 482) | |
| 3. | VHDUM-1 | VHDUM-1 | LVTVL (SEQ | CL2 |
| | | | ID NO: 483) | |
| 4. | VHDUM-1 | DOM10-53-345 | KVEIKR (SEQ | CK |
| | | | ID NO: 471) | |
| 5. | VHDUM-1 | DOM10-53-345 | KVTVL (SEQ | CL2 |
| | | | ID NO: 482) | |
| 6. | HEL-4 | HEL-4 | KVEIKR (SEQ | CK |
| | | | ID NO: 471) | |
| 7. | DOM9-112 | DOM10-53-285 | KVEIKR (SEQ | CK |
| | | | ID NO: 471) | |
| 8. | DOM9-112 | DOM10-53-347 | KVEIKR (SEQ | CK |
| | | | ID NO: 471) | |
| 9. | DOM9-112 | DOM10-53-337 | LVTVL (SEQ | CL2 |
| | | | ID NO: 483) | |
| 10. | DOM9-112 | DOM10-53-343 | KVTVL (SEQ | CL2 |
| | | | ID NO: 482) | |
| 11. | DOM9-112 | DOM10-53-343 | LVTVL | CL2 |
| | | | LVTVL (SEQ | |
| | | | ID NO: 483) | |
| 12. | DOM10-53-285 | DOM9-112 | KVEIKR (SEQ | CK |
| | | | ID NO: 471) | |
| 13. | DOM10-53-338 | DOM9-112 | KVTVL (SEQ | CL2 |
| | | | ID NO: 482) | |
| 14. | DOM10-53-338 | DOM9-112 | LVTVL | CL2 |
| 15. | DOM10-53-345 | VHDUM-1 | KVEIKR (SEQ | CK |
| | | | ID NO: 471) | |
| 16. | DOM10-53-345 | VHDUM-1 | KVTVL (SEQ | CL2 |
| | | | ID NO: 482) | |
| 17. | DOM10-53-347 | DOM9-112 | KVEIKR (SEQ | CK |
| | | | ID NO: 471) | |
| 18. | DOM10-53-347 | DOM9-112 | KVTVL (SEQ | CL2 |
| | | | ID NO: 482) | |
| 19. | DOM15-26 | DOM16-201 | KVEIKR (SEQ | CK |
| | | | ID NO: 471) | |
| 20. | DOM15-26 | DOM15-26 | KVEIKR (SEQ | CK |
| | | | ID NO: 471) | |

TABLE 8
IgGs including 4 VK variable domains expressed with natural junctions

| | | | Junction | |
|---|---|---|---|---|
| | | | between GQGT | |
| | | | in J-segment | Non-native |
| | Heavy chain | Light chain variable | and non-native | constant |
| Number | variable domain | domain | constant domain | domain |
| 21. | VKDUM-1 | VKDUM-1 | LVTVSS (SEQ | CH (IgG1) |
| | | | ID NO: 484) | |
| 22. | DOM2-100-206 | DOM15-10 | LVTVSS (SEQ | CH (IgG1) |
| | | | ID NO: 484) | |
| 23. | DOM4-122-24 | DOM4-130-54 | LVTVSS (SEQ | CH (IgG1) |
| | | | ID NO: 484) | |
| 24. | DOM4-130-54 | DOM4-122-24 | LVTVSS (SEQ | CH (IgG1) |
| | | | ID NO: 484) | |
| 25. | DOM4-130-54 | DOM4-130-54 | LVTVSS (SEQ | CH (IgG1) |
| | | | ID NO: 484) | |
| 26. | DOM9-155-25 | DOM10-176-511 | LVTVSS (SEQ | CH (IgG1) |
| | | | ID NO: 484) | |
| 27. | DOM9-155-25 | DOM10-176-535 | LVTVSS (SEQ | CH (IgG1) |
| | | | ID NO: 484) | |
| 28. | DOM9-155-25 | DOM10-176 | LVTVSS (SEQ | CH (IgG1) |
| | | | ID NO: 484) | |
| 29. | DOM9-155-25 | DOM10-176-535 | LVTVSS (SEQ | CH (IgG1) |
| | | | ID NO: 484) | |
| 30. | DOM9-155-25 | DOM10-176-535 | LVTVSS (SEQ | CH (IgG1) |
| | | | ID NO: 484) | |
| 31. | DOM9-155-29 | DOM10-176 | LVTVSS (SEQ | CH (IgG1) |
| | | | ID NO: 484) | |
| 32. | DOM9-155-29 | DOM10-176-535 | LVTVSS (SEQ | CH (IgG1) |
| | | | ID NO: 484) | |
| 33. | DOM9-44-502 | DOM10-176-511 | LVTVSS (SEQ | CH (IgG1) |
| | | | ID NO: 484) | |
| 34. | DOM9-44-502 | DOM10-176 | LVTVSS (SEQ | CH (IgG1) |
| | | | ID NO: 484) | |
| 35. | DOM10-176- | DOM9-44-502 | LVTVSS (SEQ | CH (IgG1) |
| | 511 | | ID NO: 484) | |
| 36. | DOM10-176- | DOM9-155-25 | LVTVSS (SEQ | CH (IgG1) |
| | 535 | | ID NO: 484) | |
| 37. | DOM15-10 | DOM15-10 | LVTVSS (SEQ | CH (IgG1) |
| | | | ID NO: 484) | |
| 38. | DOM15-10 | DOM16-200 | LVTVSS (SEQ | CH (IgG1) |
| | | | ID NO: 484) | |
| 39. | DOM15-10 | DOM16-32 | LVTVSS (SEQ | CH (IgG1) |
| | | | ID NO: 484) | |
| 40. | DOM15-10 | DOM16-72 | LVTVSS (SEQ | CH (IgG1) |
| | | | ID NO: 484) | |
| 41. | DOM15-10 | DOM16-39 | LVTVSS (SEQ | CH (IgG1) |
| | | | ID NO: 484) | |
| 42. | DOM15-10 | DOM2-100-206 | LVTVSS (SEQ | CH (IgG1) |
| | | | ID NO: 484) | |
| 43. | DOM16-200 | DOM16-200 | LVTVSS (SEQ | CH (IgG1) |
| | | | ID NO: 484) | |
| 44. | DOM16-32 | DOM15-10 | LVTVSS (SEQ | CH (IgG1) |
| | | | ID NO: 484) | |
| 45. | DOM16-39 | DOM16-39 | LVTVSS (SEQ | CH (IgG1) |
| | | | ID NO: 484) | |
| 46. | DOM16-39 | DOM15-10 | LVTVSS (SEQ | CH (IgG1) |
| | | | ID NO: 484) | |
| 47. | DOM16-72 | DOM15-10 | LVTVSS (SEQ | CH (IgG1) |
| | | | ID NO: 484) | |

TABLE 9
“inside-out” IgGs expressed with natural junctions

| | | | Junction | |
|---|---|---|---|---|
| | | | between GQGT | |
| | | | in J-segment | Non-native |
| | Heavy chain | Light chain variable | and non-native | constant |
| Number | variable domain | domain | constant domain | domain |
| 48. | DOM15-10 | DOM15-26 | LVTVSS (SEQ | CH (IgG1) & |
| | | | ID NO: 484 & | CK |
| | | | KVEIKR (SEQ | |
| | | | ID NO: 471) | |

In Tables 7-9, the non-native constant domain referred to in the right column is CH (IgG1) for IgGs comprising 2 Vκ variable domains, and either Cκ or Cλ2 for IgGs comprising 2 VH variable domains. For IgG 50 both constant domain sequences are non-native as this was an inside-out IgG with a VH variable domain fused to Cκ via the sequence KVEIKR and a Vκ variable domain fused to CH (IgG1) via the sequence LVTVSS.
Sequences of non-native constant domains:

CH (IgG1) (SEQ ID NO: 517):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
CK (SEQ ID NO: 518):
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC
CL2 (SEQ ID NO: 519):
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A recombinant fusion protein comprising a hybrid domain, wherein said hybrid domain comprises a first portion derived from a first polypeptide and a second portion derived from a second polypeptide, said first polypeptide comprising a domain that has the formula (X1-Y-X2), and said second polypeptide comprising a domain that has the formula (Z1-Y-Z2), wherein
Y is a conserved amino acid motif;
X1 and Z1 are the amino acid motifs that are located adjacent to the amino-terminus of Y in said first polypeptide and said second polypeptide, respectively;
X2 and Z2 are the amino acid motifs that are located adjacent to the carboxy-terminus of Y in said first polypeptide and said second polypeptide, respectively;
with the proviso that when the amino acid sequences of X1 and Z1 are the same, the amino acid sequences of X2 and Z2 are not the same; and when the amino acid sequences of X2 and Z2 are the same, the amino acid sequences of X1 and Z1 are not the same;
wherein said hybrid domain has the formula
(X1-Y-Z2).

2. The recombinant fusion protein of claim 1, wherein said hybrid domain is bonded to an amino-terminal amino acid sequence D, and/or bonded to a carboxy-terminal amino acid sequence E, such that the recombinant fusion protein comprises a structure that has the formula
D-(X1-Y-Z2)-E;
wherein D is absent or is an amino acid sequence that is adjacent to the amino-terminus of (X1-Y-X2) in said first polypeptide; and
E is absent or is an amino acid sequence that is adjacent to the carboxy-terminus of (Z1-Y-Z2) in said second polypeptide.

3. The recombinant fusion protein of claim 2, wherein D is present.

4. The recombinant fusion protein of claim 2, wherein E is present.

5. The recombinant fusion protein of claim 2, wherein D and E are present.

6. The recombinant fusion protein of claim 1, wherein (X1-Y-Z2) is a hybrid immunoglobulin variable domain.

7. The recombinant fusion protein of claim 6, wherein said hybrid immunoglobulin variable domain is a hybrid antibody variable domain.

8. The recombinant fusion protein of claim 7, wherein Y is in framework region (FR) 4.

9. The recombinant fusion protein of claim 8, wherein Y is GlyXaaGlyThr or GlyXaaGlyThrXaa(Val/Leu).

10. The recombinant fusion protein of claim 8, wherein X1 is a portion of an antibody variable domain comprising FR1, complementarity determining region (CDR) 1, FR2, CDR2, FR3, and CDR3.

11. The recombinant fusion protein of claim 7, wherein Y is in FR3.

12. The recombinant fusion protein of claim 11, wherein Y is GluAspThrAla, ValTyrTyrCys, or GluAspThrAlaValTyrTyrCys.

13. The recombinant fusion protein of claim 11, wherein X1 is a portion of an antibody variable domain comprising FR1, CDR1, FR2, and CDR2.

14. The recombinant fusion protein of claim 1, wherein (X1-Y-Z2) is a hybrid immunoglobulin constant domain.

15. The recombinant fusion protein of claim 14, wherein said hybrid immunoglobulin constant domain is a hybrid antibody constant domain.

16. The recombinant fusion protein of claim 15, wherein Y is (Ser/Ala/Gly)Pro(Lys/Asp/Ser)Val, (Ser/Ala/Gly)Pro(Lys/Asp/Ser)ValPhe, LysValAspLys(Ser/Arg/Thr) or ValThrVal.

17. The recombinant fusion protein of claim 16, wherein Y is selected from the group consisting of SerProLysVal, SerProAspVal, SerProSerVal, AlaProLysVal, AlaProAspVal, AlaProSerVal, GlyProLysVal, GlyProAspVal, GlyProSerVal, SerProLysValPhe, SerProAspValPhe, SerProSerValPhe, AlaProLysValPhe, AlaProAspValPhe, AlaProSerValPhe, GlyProLysValPhe, GlyProAspValPhe, GlyProSerValPhe, LysValAspLysSer, LysValAspLysArg, LysValAspLysThr, and or ValThrVal.

18. The recombinant fusion protein of claim 2, wherein D is absent, (X1-Y-Z2) is a hybrid immunoglobulin variable domain, and E is an immunoglobulin constant domain.

19. The recombinant fusion protein of claim 18, further comprising a second immunoglobulin variable domain that is amino terminal to (X1-Y-Z2).

20. The recombinant fusion protein of claim 2, wherein D is an immunoglobulin variable domain, and (X1-Y-Z2) is a hybrid immunoglobulin constant domain.

21. The recombinant fusion protein of claim 2, wherein (X1-Y-Z2) is a hybrid immunoglobulin constant domain, and E is an immunoglobulin constant domain.

22. The recombinant fusion protein of claim 21, wherein D is absent and the fusion protein comprises a further domain that is amino terminal to (X1-Y-Z2).

23. The recombinant fusion protein of claim 2, wherein D is an immunoglobulin constant domain, and (X1-Y-Z2) is a hybrid immunoglobulin constant domain.

24. The recombinant fusion protein of claim 1, wherein said first polypeptide and said second polypeptide are both members of the same protein superfamily.

25. The recombinant fusion protein of claim 1, wherein said protein superfamily is selected from the group consisting of the immunoglobulin superfamily, the TNF superfamily and the TNF receptor superfamily.

26. The recombinant fusion protein of claim 1, wherein said first polypeptide and said second polypeptide are both human polypeptides.

27. The recombinant fusion protein of claim 1, wherein X1, X2, Z1 and Z2 each, independently, consists of about 1 to about 200 amino acids.

28. The recombinant fusion protein of claim 1, wherein said hybrid domain is about the size of an immunoglobulin variable domain.

29. The recombinant fusion protein of claim 1, wherein said hybrid domain is about the size of an immunoglobulin constant domain.

30. The recombinant fusion protein of claim 1, wherein said hybrid domain is about 8 kDa to about 20 kDa.

31. An isolated recombinant nucleic acid molecule encoding the recombinant fusion protein of claim 1.

32. A host cell comprising a recombinant nucleic acid molecule encoding the recombinant fusion protein of claim 1.

33. A method of producing a recombinant fusion protein comprising maintaining the host cell of claim 32 under conditions suitable for expression of said recombinant nucleic acid, whereby said recombinant nucleic acid is expressed and said recombinant fusion protein is produced.

34. The method of claim 33, further comprising isolating said recombinant fusion protein.

35. A recombinant fusion protein comprising a hybrid immunoglobulin variable domain that is fused to an immunoglobulin constant domain, wherein said hybrid immunoglobulin variable domain comprises a hybrid framework region (FR) that comprises a portion from a first immunoglobulin FR from a first immunoglobulin and a portion from a second immunoglobulin FR from a second immunoglobulin, said first immunoglobulin FR and said second immunoglobulin FR each comprising a conserved amino acid motif Y, and said hybrid immunoglobulin FR has the formula
(F¹-Y-F²)
wherein Y is said conserved amino acid motif;
F¹ is the amino acid motif located adjacent to the amino-terminus of Y in said first immunoglobulin FR; and
F² is the amino acid motif located adjacent to the carboxy-terminus of Y in said second immunoglobulin FR.

36. The recombinant fusion protein of claim 35, wherein Y is located in framework region (FR) 1, FR2 or FR3 of said first immunoglobulin and of said second immunoglobulin.

37. The recombinant fusion protein of claim 35, wherein Y is located in FR4 of said first immunoglobulin and of said second immunoglobulin.

38. The recombinant fusion protein of claim 35, wherein said hybrid FR is a hybrid FR4, and F² is adjacent to the amino-terminus of said immunoglobulin constant domain in a naturally occurring protein comprising said immunoglobulin constant domain.

39. The recombinant fusion protein of claim 35, wherein said immunoglobulin constant domain is a T cell receptor constant domain and said second immunoglobulin FR is a FR4 from a T cell receptor variable domain.

40. The recombinant fusion protein of claim 39, wherein F² is amino terminal to said immunoglobulin constant domain in a naturally occurring immunoglobulin.

41. The recombinant fusion protein of claim 35, wherein said immunoglobulin constant domain is an antibody light chain constant domain and said second immunoglobulin FR is a FR4 from an antibody light chain variable domain.

42. The recombinant fusion protein of claim 41, wherein F² is amino terminal to said antibody light chain constant domain in a naturally occurring antibody light chain.

43. The recombinant fusion protein of claim 41, wherein said antibody constant domain is a Cκ or Cλ, and said second antibody FR4 is a Vκ FR4 or Vλ FR4, respectively.

44. The recombinant fusion protein of claim 43, wherein said first antibody variable domain is an antibody heavy chain variable domain.

45. The recombinant fusion protein of claim 35, wherein said immunoglobulin constant domain is an antibody heavy chain constant domain and said second immunoglobulin FR is a FR4 from an antibody heavy chain variable domain.

46. The recombinant fusion protein of claim 35, wherein said first immunoglobulin is a non-human immunoglobulin.

47. The recombinant fusion protein of claim 46, wherein said non-human immunoglobulin is an immunoglobulin from a mouse, rat, shark, fish, possum, sheep, pig, Camelid, rabbit or non-human primate.

48. The recombinant fusion protein of claim 47, wherein said non-human immunoglobulin is a Camelid or nurse shark heavy chain antibody.

49. The recombinant fusion protein of claim 46, wherein said second immunoglobulin is a human immunoglobulin.

50. The recombinant fusion protein of claim 35, wherein said immunoglobulin constant domain is a human immunoglobulin constant domain.

51. The recombinant fusion protein of claim 35, wherein said hybrid immunoglobulin variable domain is a hybrid antibody variable domain.

52. The recombinant fusion protein of claim 51, wherein Y is GlyXaaGlyThr.

53. The recombinant fusion protein of claim 52, wherein F¹ is Phe and F² is (Leu/Met/Thr)ValThrValSerSer.

54. The recombinant fusion protein of claim 53, wherein F² is selected from the group consisting of LeuValThrValSerSer, MetValThrValSerSer; and ThrValThrValSerSer.

55. The recombinant fusion protein of claim 53, wherein said immunoglobulin constant domain is a human antibody constant domain.

56. The recombinant fusion protein of claim 55, wherein said human antibody constant domain is an IgG CH1 domain.

57. The recombinant fusion protein of claim 52, wherein said hybrid antibody variable domain is a hybrid heavy chain variable domain, F¹ is Trp and F² is (Lys/Arg)(Val/Leu)(Glu/Asp)IleLys or (Lys/Gln/Glu)(Val/Leu)(Thr/Ile)(Val/Ile)Leu.

58. The recombinant fusion protein of claim 57, wherein F² is selected from the group consisting of LysValGluIleLys, LysValAspIleLys, LysLeuGluIleLys, LysLeuAspIleLys, ArgValGluIleLys, ArgValAspIleLys, ArgLeuGluIleLys, ArgLeuAspIleLys, LysValThrValLeu, LysValThrIleLeu, LysValIleValLeu, LysValIleIleLeu, LysLeuThrValLeu, LysLeuThrIleLeu, LysLeuIleValLeu, LysLeuIleIleLeu, GlnValThrValLeu, GlnValThrIleLeu, GlnValIleValLeu, GlnValIleIleLeu, GlnLeuThrValLeu, GlnLeuThrIleLeu, GlnLeuIleValLeu, GlnLeuIleIleLeu, GluValThrValLeu, GluValThrIleLeu, GluValIleValLeu, GluValIleIleLeu, GluLeuThrValLeu, GluLeuThrIleLeu, GluLeuIleValLeu, and GluLeuIleIleLeu.

59. The recombinant fusion protein of claim 57, wherein said antibody constant domain is a human antibody light chain constant domain.

60. The recombinant fusion protein of claim 51, wherein Y is GlyXaaGlyThrXaa(Val/Leu).

61. The recombinant fusion protein of claim 60, wherein F¹ is Phe and F² is ThrValSerSer.

62. The recombinant fusion protein of claim 61, wherein said antibody constant domain is a human antibody constant domain.

63. The recombinant fusion protein of claim 62, wherein said human antibody constant domain is an IgG CH1 domain or an IgG CH2 domain.

64. The recombinant fusion protein of claim 63, wherein said IgG is IgG1 or IgG4.

65. The recombinant fusion protein of claim 60, wherein F¹ is Trp and F² is (Glu/Asp)IleLys or (Thr/Ile)(Val/Ile)Leu.

66. The recombinant fusion protein of claim 65, wherein F² is selected from the group consisting of GluIleLys, AspIleLys, ThrValLeu, ThrIleLeu, IleValLeu, and IleIleLeu.

67. The recombinant fusion protein of claim 65, wherein said antibody constant domain is a human antibody light chain constant domain.

68. The recombinant fusion protein of claim 31, wherein said recombinant fusion protein comprises a partial structure that has the formula (F¹-Y-F 2)-C_{L}, (F¹-Y-F 2)-CH1, (F¹-Y-F²)-CH2, or (F¹-Y-F²)-Fc.

69. The recombinant fusion protein of claim 68, wherein said recombinant fusion protein further comprises a second immunoglobulin variable domain.

70. The recombinant fusion protein of claim 69, wherein said second immunoglobulin variable domain is amino-terminal of (F¹-Y-F²).

71. The recombinant fusion protein of claim 69, wherein said second immunoglobulin variable domain is carboxy-terminal of (F¹-Y-F²).

72. An isolated recombinant nucleic acid molecule encoding the recombinant fusion protein of claim 35.

73. A host cell comprising a recombinant nucleic acid molecule encoding the recombinant fusion protein of claim 35.

74. A method of producing a recombinant fusion protein comprising maintaining the host cell of claim 73 under conditions suitable for expression of said recombinant nucleic acid, whereby said recombinant nucleic acid is expressed and said recombinant fusion protein is produced.

75. The method of claim 74, further comprising isolating said recombinant fusion protein.

76. In a recombinant fusion protein comprising a non-human antibody variable region fused to a human antibody constant domain, the improvement comprising:
said non-human antibody variable region comprising a hybrid FR4 having the formula
(F¹-Y-F²)
wherein F¹ is Phe or Trp;
Y is GlyXaaGlyThr, and F² is (Leu/Met/Thr)ValThrValSerSer, (Lys/Arg)(Val/Leu)(Glu/Asp)IleLys or (Lys/Gln/Glu)(Val/Leu)(Thr/Ile)(Val/Ile)Leu; or
Y is GlyXaaGlyThrXaa(Val/Leu), and F² is ThrValSerSer, (Glu/Asp)IleLys or (Thr/Ile)(Val/Ile)Leu.

77-87. (canceled)

88. A recombinant fusion protein comprising an immunoglobulin variable domain fused to a hybrid immunoglobulin constant domain, wherein said hybrid immunoglobulin constant domain comprises a portion from a first immunoglobulin constant domain and a portion from a second immunoglobulin constant domain, said first immunoglobulin constant domain and said second immunoglobulin constant domain each comprising a conserved amino acid motif Y, said hybrid immunoglobulin constant domain having the formula
C¹-Y-C²
wherein Y is said conserved amino acid motif;
C¹ is the amino acid motif adjacent to the amino-terminus of Y in said first immunoglobulin constant region;
C² is the amino acid motif adjacent to the carboxy-terminus of Y in said second immunoglobulin constant region.

89-117. (canceled)

118. An isolated recombinant nucleic acid molecule encoding the recombinant fusion protein of claim 88.

119. A host cell comprising a recombinant nucleic acid molecule encoding the recombinant fusion protein of claim 88.

120. A method of producing a recombinant fusion protein comprising maintaining the host cell of claim 119 under conditions suitable for expression of said recombinant nucleic acid, whereby said recombinant nucleic acid is expressed and said recombinant fusion protein is produced.

121. (canceled)

122. A recombinant fusion protein comprising a first portion derived from a first polypeptide and a second portion derived from a second polypeptide, wherein said first polypeptide comprises a structure having the formula (A)-L1, wherein
(A) is an amino acid sequence present in said first polypeptide; and
L1 is an amino acid motif comprising 1 to about 50 amino acids that are adjacent to the carboxy-terminus of (A) in said first polypeptide;
wherein said fusion polypeptide has the formula
(A)-L1-(B);
wherein (B) is said portion derived from said second polypeptide;
with the proviso that at least one of (A) and (B) is a domain, and when (A) and (B) are both antibody variable domains
a) (A) and (B) are each human antibody variable domains;
b) (A) and (B) are each antibody heavy chain variable domains;
c) (A) and (B) are each antibody light chain variable domains;
d) (A) is an antibody light chain variable domain and (B) is an antibody heavy chain variable domain; or
e) (A) is a VHH and (B) is an antibody light chain variable domain; or
with the proviso that when (A) and (B) are both antibody variable domains the following is excluded from the invention, (A)-L1-(B) where (A) is a mouse VH, (B) is a mouse VL and L1 is SerAlaLysThrThrPro, SerAlaLysThrThrProLysLeuGlyGly, AlaLysThrThrProLysLeuGluGluGlyGluPheSerGluAlaArgVal, or AlaLysThrThrProLysLeuGluGlu.

123-149. (canceled)

150. An isolated recombinant nucleic acid molecule encoding the recombinant fusion protein of claim 122.

151. A host cell comprising a recombinant nucleic acid molecule encoding the recombinant fusion protein of claim 122.

152. A method of producing a recombinant fusion protein comprising maintaining the host cell of claim 151 under conditions suitable for expression of said recombinant nucleic acid, whereby said recombinant nucleic acid is expressed and said recombinant fusion protein is produced.

153. (canceled)

154. A recombinant fusion protein comprising a first portion that is an immunoglobulin variable domain and a second portion, wherein said first portion is bonded to said second portion through a linker, and the recombinant fusion protein has the formula
(A′)-L2-(B)
wherein (A′) is said immunoglobulin variable domain and comprises framework (FR) 4;
L2 is said linker, wherein L2 comprises one to about 50 contiguous amino acids that are adjacent to the carboxy-terminus of said FR4 in a naturally occurring immunoglobulin that comprises said FR4; and
(B) is said second portion;
with the proviso that L2-(B) is not a C_{L} or CH1 domain that is peptide bonded to said FR4 in a naturally occurring antibody that comprises said FR4, and when (A) and (B) are both antibody variable domains
a) (A) and (B) are each human antibody variable domains;
b) (A) and (B) are each antibody heavy chain variable domains;
c) (A) and (B) are each antibody light chain variable domains;
d) (A) is an antibody light chain variable domain and (B) is an antibody heavy chain variable domain; or
e) (A) is a VHH and (B) is an antibody light chain variable domain or
with the proviso that when (A) and (B) are both antibody variable domains the following is excluded from the invention, (A)-L1-(B) where (A) is a mouse VH, (B) is a mouse VL and L1 is SerAlaLysThrThrPro, SerAlaLysThrThrProLysLeuGlyGly, AlaLysThrThrProLysLeuGluGluGlyGluPheSerGluAlaArgVal, or AlaLysThrThrProLysLeuGlyGly.

155-182. (canceled)

183. A recombinant fusion protein comprising a first portion and a second portion derived from an immunoglobulin constant region, wherein said first portion is bonded to said second portion through a linker, and the recombinant fusion protein has the formula
(A)-L3-(C³)
wherein (A) is said first portion;
(C³) is said second portion derived from an immunoglobulin constant region; and
L3 is said linker, wherein L3 comprises one to about 50 contiguous amino acids that are adjacent to the amino-terminus of (C³) in a naturally occurring immunoglobulin that comprises (C³);
with the proviso that (A) is not an antibody variable domain found in said naturally occurring immunoglobulin.

184-208. (canceled)

209. A recombinant fusion protein comprising a first portion derived from an antibody variable domain and a second portion derived from a second polypeptide, wherein said antibody variable domain comprises a structure having the formula (A)-L1, wherein
(A) consists of CDR3; and
L1 consists of FR4;
wherein said fusion polypeptide has the formula (A)-L1-(B);
wherein
(B) is said portion derived from said second polypeptide.

210-211. (canceled)

212. An isolated recombinant nucleic acid molecule encoding the recombinant fusion protein of claim 154.

213. A host cell comprising a recombinant nucleic acid molecule encoding the recombinant fusion protein of claim 154.

214. A method of producing a recombinant fusion protein comprising maintaining the host cell of claim 213 under conditions suitable for expression of said recombinant nucleic acid, whereby said recombinant nucleic acid is expressed and said recombinant fusion protein is produced.

215-217. (canceled)

218. A method of therapy, diagnosis and/or prophylaxis in a human comprising administering to said human an effective amount of a recombinant fusion protein of claim 1, whereby the likelihood of inducing an immune response is reduced in comparison to a corresponding fusion protein that does not contain a natural junction.

219-225. (canceled)

226. A pharmaceutical composition comprising a recombinant fusion protein of claim 1 and a physiologically acceptable carrier.

227. A method of producing a fusion protein comprising a first portion and a second portion that are fused at a natural junction, wherein said first portion is derived from a first polypeptide and said second portion is derived from a second polypeptide, the method comprising, analyzing the amino acid sequence of said first polypeptide or a portion thereof and the amino acid sequence of said second polypeptide or a portion thereof to identify a conserved amino acid motif present in both of the analyzed sequences; and preparing a fusion protein which has the formula
A-Y-B;
wherein:
A is said first portion;
Y is said conserved amino acid motif;
B is said second portion; and
wherein said first polypeptide comprises A-Y, and said second polypeptide comprises Y-B.

228-275. (canceled)


SEQUENCE LISTING (542 sequences)

SEQ ID NO: 1 - protein, 17 aa, Homo sapiens
AEYFQHWGQGTLVTVSS

SEQ ID NO: 2 - protein, 17 aa, Homo sapiens
YWYFDLWGRGTLVTVSS

SEQ ID NO: 3 - protein, 15 aa, Homo sapiens
AFDVWGQGTMVTVSS

SEQ ID NO: 4 - protein, 15 aa, Homo sapiens
YFDYWGQGTLVTVSS

SEQ ID NO: 5 - protein, 16 aa, Homo sapiens
NWFDSWGQGTLVTVSS

SEQ ID NO: 6 - protein, 20 aa, Homo sapiens
YYYYYGMDVWGQGTTVTVSS

SEQ ID NO: 7 - protein, 12 aa, Homo sapiens
WTFGQGTKVEIK

SEQ ID NO: 8 - protein, 12 aa, Homo sapiens
YTFGQGTKLEIK

SEQ ID NO: 9 - protein, 12 aa, Homo sapiens
FTFGPGTKVDIK

SEQ ID NO: 10 - protein, 12 aa, Homo sapiens
LTFGGGTKVEIK

SEQ ID NO: 11 - protein, 12 aa, Homo sapiens
ITFGQGTRLEIK

SEQ ID NO: 12 - protein, 12 aa, Homo sapiens
YVFGTGTKVTVL

SEQ ID NO: 13 - protein, 12 aa, Homo sapiens
VVFGGGTKLTVL

SEQ ID NO: 14 - protein, 12 aa, Homo sapiens
VVFGGGTKLTVL

SEQ ID NO: 15 - protein, 12 aa, Homo sapiens
FVFGGGTQLIIL

SEQ ID NO: 16 - protein, 12 aa, Homo sapiens
WVFGEGTELTVL

SEQ ID NO: 17 - protein, 12 aa, Homo sapiens
NVFGSGTKVTVL

SEQ ID NO: 18 - protein, 12 aa, Homo sapiens
AVFGGGTQLTVL

SEQ ID NO: 19 - protein, 15 aa, Homo sapiens
NTEAFFGQGTRLTVV

SEQ ID NO: 20 - protein, 15 aa, Homo sapiens
NYGYTFGSGTRLTVV

SEQ ID NO: 21 - protein, 16 aa, Homo sapiens
SGNTIYFGEGSWLTVV

SEQ ID NO: 22 - protein, 16 aa, Homo sapiens
TNEKLFFGSGTQLSVL

SEQ ID NO: 23 - protein, 16 aa, Homo sapiens
SNQPQHFGDGTRLSIL

SEQ ID NO: 24 - protein, 17 aa, Homo sapiens
SYNSPLHFGNGTRLTVT

SEQ ID NO: 25 - protein, 16 aa, Homo sapiens
SYNEQFFGPGTRLTVL

SEQ ID NO: 26 - protein, 16 aa, Homo sapiens
NTGELFFGEGSRLTVL

SEQ ID NO: 27 - protein, 15 aa, Homo sapiens
LRGAAGRLGGGLLVL

SEQ ID NO: 28 - protein, 16 aa, Homo sapiens
STDTQYFGPGTRLTVL

SEQ ID NO: 29 - protein, 16 aa, Homo sapiens
AKNIQYFGAGTRLSVL

SEQ ID NO: 30 - protein, 15 aa, Homo sapiens
QETQYFGPGTRLLVL

SEQ ID NO: 31 - protein, 17 aa, Homo sapiens
SGANVLTFGAGSRLTVL

SEQ ID NO: 32 - protein, 15 aa, Homo sapiens
SYEQYFGPGTRLTVT

SEQ ID NO: 33 - protein, 16 aa, Homo sapiens
NYYKKLFGSGTTLVVT

SEQ ID NO: 34 - protein, 16 aa, Homo sapiens
NYYKKLFGSGTTLVVT

SEQ ID NO: 35 - protein, 20 aa, Homo sapiens
GQELGKKIKVFGPGTKLIIT

SEQ ID NO: 36 - protein, 19 aa, Homo sapiens
TTGWFKIFAEGTKLIVTSP

SEQ ID NO: 37 - protein, 19 aa, Homo sapiens
SSDWIKTFAKGTRLIVTSP

SEQ ID NO: 38 - protein, 16 aa, Homo sapiens
TDKLIFGKGTRVTVEP

SEQ ID NO: 39 - protein, 17 aa, Homo sapiens
LTAQLFFGKGTQLIVEP

SEQ ID NO: 40 - protein, 19 aa, Homo sapiens
SWDTRQMFFGTGIKLFVEP

SEQ ID NO: 41 - protein, 15 aa, Homo sapiens
RPLIFGKGTYLEVQQ

SEQ ID NO: 42 - protein, 19 aa, Homo sapiens
YRVNRKLTFGANTRGIMKL

SEQ ID NO: 43 - protein, 17 aa, Homo sapiens
KEGNRKFTFGMGTQVRV

SEQ ID NO: 44 - protein, 19 aa, Homo sapiens
ETSGSRLTFGEGTQLTVNP

SEQ ID NO: 45 - protein, 20 aa, Homo sapiens
TQGGSEKLVFGKGTKLTVNP

SEQ ID NO: 46 - protein, 20 aa, Homo sapiens
YTGANSKLTFGKGITLSVRP

SEQ ID NO: 47 - protein, 19 aa, Homo sapiens
IQGAQKLVFGQGTRLTINP

SEQ ID NO: 48 - protein, 21 aa, Homo sapiens
NSGGSNYKLTFGKGTLLTVNP

SEQ ID NO: 49 - protein, 22 aa, Homo sapiens
NAGGTSYGKLTFGQGTILTVHP

SEQ ID NO: 50 - protein, 19 aa, Homo sapiens
KTSYDKVIFGPGTSLSVIP

SEQ ID NO: 51 - protein, 18 aa, Homo sapiens
NTGNQFYFGTGTSLTVIP

SEQ ID NO: 52 - protein, 20 aa, Homo sapiens
SNFGNEKLTFGTGTRLTIIP

SEQ ID NO: 53 - protein, 18 aa, Homo sapiens
EYGNKLVFGAGTILRVKS

SEQ ID NO: 54 - protein, 20 aa, Homo sapiens
KKSSGDKLTFGTGTRLAVRP

SEQ ID NO: 55 - protein, 21 aa, Homo sapiens
YSGGGADGLTFGKGTHLIIQP

SEQ ID NO: 56 - protein, 20 aa, Homo sapiens
NTGTASKLTFGTGTRLQVTL

SEQ ID NO: 57 - protein, 17 aa, Homo sapiens
NNNDMRFGAGTRLTVKP

SEQ ID NO: 58 - protein, 20 aa, Homo sapiens
NSNSGYALNFGKGTSLLVTP

SEQ ID NO: 59 - protein, 20 aa, Homo sapiens
TTSGTYKYIFGTGTRLKVLA

SEQ ID NO: 60 - protein, 20 aa, Homo sapiens
NNNAGNMLTFGGGTRLMVKP

SEQ ID NO: 61 - protein, 20 aa, Homo sapiens
NAGNNRKLIWGLGTSLAVNP

SEQ ID NO: 62 - protein, 20 aa, Homo sapiens
GSGNTGKLIFGQGTTLQVKP

SEQ ID NO: 63 - protein, 19 aa, Homo sapiens
QTGANNLFFGTGTRLTVIP

SEQ ID NO: 64 - protein, 19 aa, Homo sapiens
IGFGNVLHCGSGTQVIVLP

SEQ ID NO: 65 - protein, 19 aa, Homo sapiens
SYNTDKLIFGTGTRLQVFP

SEQ ID NO: 66 - protein, 18 aa, Homo sapiens
DSNYQLIWGAGTKLIIKP

SEQ ID NO: 67 - protein, 21 aa, Homo sapiens
NYGGATNKLIFGTGTLLAVQP

SEQ ID NO: 68 - protein, 18 aa, Homo sapiens
NNNARLMFGDGTQLVVKP

SEQ ID NO: 69 - protein, 18 aa, Homo sapiens
NRDDKIIFGKGTRLHILP

SEQ ID NO: 70 - protein, 19 aa, Homo sapiens
NSGNTPLVFGKGTRLSVIA

SEQ ID NO: 71 - protein, 21 aa, Homo sapiens
YSGAGSYQLTFGKGTKLSVIP

SEQ ID NO: 72 - protein, 19 aa, Homo sapiens
NTNAGKSTFGDGTTLTVKP

SEQ ID NO: 73 - protein, 19 aa, Homo sapiens
DNYGQNFVFGPGTRLSVLP

SEQ ID NO: 74 - protein, 19 aa, Homo sapiens
EGQGFSFIFGKGTRLLVKP

SEQ ID NO: 75 - protein, 20 aa, Homo sapiens
TTDSWGKFEFGAGTQVVVTP

SEQ ID NO: 76 - protein, 20 aa, Homo sapiens
IYNQGGKLIFGQGTELSVKP

SEQ ID NO: 77 - protein, 20 aa, Homo sapiens
SSGSARQLTFGSGTQLTVLP

SEQ ID NO: 78 - protein, 18 aa, Homo sapiens
YNFNKFYFGSGTKLNVKP

SEQ ID NO: 79 - protein, 18 aa, Homo sapiens
SNDYKLSFGAGTTVTVRA

SEQ ID NO: 80 - protein, 19 aa, Homo sapiens
YQRFYNFTFGKGSKHNVTP

SEQ ID NO: 81 - protein, 21 aa, Homo sapiens
DRGSTLGRLYFGRGTQLTVWP

SEQ ID NO: 82 - protein, 20 aa, Homo sapiens
IKAAGNKLTFGGGTRVLVKP

SEQ ID NO: 83 - protein, 19 aa, Homo sapiens
FSDGQKLLFARGTMLKVDL

SEQ ID NO: 84 - protein, 19 aa, Homo sapiens
NQAGTALIFGKGTTLSVSS

SEQ ID NO: 85 - protein, 17 aa, Homo sapiens
IYSTFIFGSGTRLSVKP

SEQ ID NO: 86 - protein, 20 aa, Homo sapiens
NSGGYQKVTFGIGTKLQVIP

SEQ ID NO: 87 - protein, 19 aa, Homo sapiens
MDSSYKLIFGSGTRLLVRP

SEQ ID NO: 88 - protein, 19 aa, Homo sapiens
NSGYSTLTFGKGTMLLVSP

SEQ ID NO: 89 - protein, 21 aa, Homo sapiens
ILTGGGNKLTFGTGTQLKVEL

SEQ ID NO: 90 - protein, 20 aa, Homo sapiens
GNTGGFKTIFGAGTRLFVKA

SEQ ID NO: 91 - protein, 19 aa, Homo sapiens
NTGFQKLVFGTGTRLLVSP

SEQ ID NO: 92 - protein, 19 aa, Homo sapiens
DYGNNRLAFGKGNQVVVIP

SEQ ID NO: 93 - protein, 20 aa, Homo sapiens
ASGGSYIPTFGRGTSLIVHP

SEQ ID NO: 94 - protein, 19 aa, Homo sapiens
DTGRRALTFGSGTRLQVQP

SEQ ID NO: 95 - protein, 20 aa, Homo sapiens
FSGGYNKLIFGAGTRLAVHP

SEQ ID NO: 96 - protein, 20 aa, Homo sapiens
GYSSASKIIFGSGTRLSIRP

SEQ ID NO: 97 - protein, 21 aa, Homo sapiens
NTGGTIDKLTFGKGTHVFIIS

SEQ ID NO: 98 - protein, 20 aa, Homo sapiens
YESITSQLQFGKGTRVSTSP

SEQ ID NO: 99 - protein, 17 aa, Unknown
  Mouse
YWYFDVWGAGTTVTVSS

SEQ ID NO: 100 - protein, 15 aa, Unknown
  Mouse
YFDYWGQGTTLTVSS

SEQ ID NO: 101 - protein, 15 aa, Unknown
  Mouse
WFAYWGQGTLVTVSA

SEQ ID NO: 102 - protein, 17 aa, Unknown
  Mouse
YYAMDYWGQGTSVTVSS

SEQ ID NO: 103 - protein, 18 aa, Unknown
  Llama
GYRYLEVWGQGTLVTVSS

SEQ ID NO: 104 - protein, 16 aa, Unknown
  Llama
NALDAWGQGTLVTVSS

SEQ ID NO: 105 - protein, 15 aa, Unknown
  Llama
EYDYWGQGTQVTVSS

SEQ ID NO: 106 - protein, 15 aa, Unknown
  Llama
PQFEYWGQGTLVTVS

SEQ ID NO: 107 - protein, 14 aa, Unknown
  Llama
DFGSWGQGTLVTVS

SEQ ID NO: 108 - protein, 17 aa, Unknown
  Sheep
YADFHLWDQGALVTVSS

SEQ ID NO: 109 - protein, 17 aa, Unknown
  Sheep
CWDSGLWGQRTPVTVSL

SEQ ID NO: 110 - protein, 15 aa, Unknown
  Sheep
AFDSWGQRAPVTVSS

SEQ ID NO: 111 - protein, 15 aa, Unknown
  Sheep
YIDYWGPGLLVTVSS

SEQ ID NO: 112 - protein, 15 aa, Unknown
  Sheep
DWLKHWGQGPRRCLL

SEQ ID NO: 113 - protein, 17 aa, Unknown
  Sheep
YYGVDVWGRGLLVTVSS

SEQ ID NO: 114 - protein, 14 aa, Unknown
  Pig
LLCWGPGVEVVVSS

SEQ ID NO: 115 - protein, 12 aa, Unknown
  Mouse
WTFGGGTKLEIK

SEQ ID NO: 116 - protein, 12 aa, Unknown
  Mouse
YTFGGGTKLEIK

SEQ ID NO: 117 - protein, 12 aa, Unknown
  Mouse
ITFSDGTRLEIK

SEQ ID NO: 118 - protein, 12 aa, Unknown
  Mouse
FTFGSGTKLEIK

SEQ ID NO: 119 - protein, 12 aa, Unknown
  Mouse
LTFGAGTKLELK

SEQ ID NO: 120 - protein, 12 aa, Unknown
  Possum
LTFGQGTKLEIK

SEQ ID NO: 121 - protein, 12 aa, Unknown
  Possum
FTFGGGTKVEIK

SEQ ID NO: 122 - protein, 11 aa, Unknown
  Sheep
SFGQGTKLEIK

SEQ ID NO: 123 - protein, 12 aa, Unknown
  Sheep
FTFGPGTRVEIK

SEQ ID NO: 124 - protein, 11 aa, Unknown
  Sheep
YAFGGGTKVEI

SEQ ID NO: 125 - protein, 12 aa, Unknown
  Sheep
LAFGGGTNVEIK

SEQ ID NO: 126 - protein, 12 aa, Unknown
  Mouse
WVFGGGTKLTVL

SEQ ID NO: 127 - protein, 12 aa, Unknown
  Mouse
YVFGGGTKVTVL

SEQ ID NO: 128 - protein, 12 aa, Unknown
  Mouse
FIFGSGTKVTVL

SEQ ID NO: 129 - protein, 12 aa, Unknown
  Mouse
GSFSSNGLLYAG

SEQ ID NO: 130 - protein, 12 aa, Unknown
  Mouse
WVFGGGTRLTVL

SEQ ID NO: 131 - protein, 12 aa, Unknown
  Possum
YIFGGGTQLTVI

SEQ ID NO: 132 - protein, 12 aa, Unknown
  Possum
WVFGEGTHVTVL

SEQ ID NO: 133 - protein, 12 aa, Unknown
  Possum
WVFGGGTHLTVL

SEQ ID NO: 134 - protein, 12 aa, Unknown
  Sheep
GVFGSGTRLTVL

SEQ ID NO: 135 - protein, 101 aa, Unknown
  Mouse
ESARNPTIYPLTLPPVLCSDPVIIGCLIHDYFPFGTMNVTWGKSGKDITTVNFPPALASGGRYTMSSQLTLPAVECPEGESVKCSVQHDSNPVQELDVNCS

SEQ ID NO: 136 - protein, 94 aa, Unknown
  Mouse
GDKKEPDMFLLSECKAPEENEKINLGCLVIGSQPLKISWEPKKSSIVEHVFPSEMRNGNYTMVLQVTVLASELNLNHTCTINKPKRKEKPFKFP

SEQ ID NO: 137 - protein, 91 aa, Unknown
  Mouse
ASIRNPQLYPLKPCKGTASMTLGCLVKDYFPNPVTVTWYSDSLNMSTVNFPALGSELKVTTSQVTSWGKSAKNFTCHVTHPPSFNESRTIL

SEQ ID NO: 138 - protein, 97 aa, Unknown
  Mouse
AKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKI

SEQ ID NO: 139 - protein, 97 aa, Unknown
  Mouse
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 140 - protein, 97 aa, Unknown
  Mouse
AKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKL

SEQ ID NO: 141 - protein, 97 aa, Unknown
  Mouse
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKI

SEQ ID NO: 142 - protein, 97 aa, Unknown
  Mouse
ATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRI

SEQ ID NO: 143 - protein, 105 aa, Unknown
  Mouse
ESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIP

SEQ ID NO: 144 - protein, 101 aa, Unknown
  Mouse
SCQPSLSLQRPALEDLLLGSDASITCTLNGLRNPEGAAFTWEPSTGKDAVQKKAAQNSCGCYSVSSVLPGCAERWNSGASFKCTVTHPESGTLTGTIAKVT

SEQ ID NO: 145 - protein, 107 aa, Unknown
  Mouse
VRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCP

SEQ ID NO: 146 - protein, 107 aa, Unknown
  Mouse
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTK

SEQ ID NO: 147 - protein, 110 aa, Unknown
  Mouse
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 148 - protein, 110 aa, Unknown
  Mouse
APNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIK

SEQ ID NO: 149 - protein, 110 aa, Unknown
  Mouse
APDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPR

SEQ ID NO: 150 - protein, 110 aa, Unknown
  Mouse
PGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPK

SEQ ID NO: 151 - protein, 113 aa, Unknown
  Mouse
AVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAA

SEQ ID NO: 152 - protein, 131 aa, Unknown
  Mouse
VNTFPPQVHLLPPPSEELALNELLSLTCLVRAFNPKEVLVRWLHGNEELSPESYLVFEPLKEPGEGATTYLVTSVLRVSAETWKQGDQYSCMVGHEALPMNFTQKTIDRLSGKPTNVSVSVIMSEGDGICY

SEQ ID NO: 153 - protein, 129 aa, Unknown
  Mouse
GAMAPSNLTVNILTTSTHPEMSSWLLCEVSGFFPENIHLMWLGVHSKMKSTNFVTANPTAQPGGTFQTWSVLRLPVALSSSLDTYTCVVEHEASKTKLNASKSLAISGCYHLLPESDGPSRRPDGPALA

SEQ ID NO: 154 - protein, 107 aa, Unknown
  Mouse
DHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTP

SEQ ID NO: 155 - protein, 107 aa, Unknown
  Mouse
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 156 - protein, 107 aa, Unknown
  Mouse
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 157 - protein, 107 aa, Unknown
  Mouse
GLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 158 - protein, 107 aa, Unknown
  Mouse
GPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 159 - protein, 107 aa, Unknown
  Mouse
GRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 160 - protein, 106 aa, Unknown
  Mouse
SPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPN

SEQ ID NO: 161 - protein, 11 aa, Unknown
  Mouse
GPTPPPPITIP

SEQ ID NO: 162 - protein, 12 aa, Unknown
  Mouse
GICSPPTTPPPP

SEQ ID NO: 163 - protein, 11 aa, Unknown
  Mouse
GPPPPCPPCPP

SEQ ID NO: 164 - protein, 8 aa, Unknown
  Mouse
GPPPPCPP

SEQ ID NO: 165 - protein, 10 aa, Unknown
  Mouse
GPPPPCPPLP

SEQ ID NO: 166 - protein, 35 aa, Unknown
  Mouse
ESWDSQSSKRVTPTLQAKNHSTEATKAITTKKDIE

SEQ ID NO: 167 - protein, 13 aa, Unknown
  Mouse
VPRDCGCKPCICT

SEQ ID NO: 168 - protein, 16 aa, Unknown
  Mouse
EPRGPTIKPCPPCKCP

SEQ ID NO: 169 - protein, 22 aa, Unknown
  Mouse
EPSGPISTINPCPPCKECHKCP

SEQ ID NO: 170 - protein, 21 aa, Unknown
  Mouse
EPRVPITQNPCPPLKECPPCA

SEQ ID NO: 171 - protein, 16 aa, Unknown
  Mouse
EPRIPKPSTPPGSSCP

SEQ ID NO: 172 - protein, 107 aa, Unknown
  Mouse
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 173 - protein, 107 aa, Unknown
  Mouse
RADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 174 - protein, 106 aa, Unknown
  Mouse
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 175 - protein, 105 aa, Unknown
  Mouse
GQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 176 - protein, 105 aa, Unknown
  Mouse
GQPKSTPTLTMFPPSPEELQENKATLVCLISNFSPSGVTVAWKANGTPITQGVDTSNPTKEDNKYMASSFLHLTSDQWRSHNSFTCQVTHEGDTVEKSLSPAECL

SEQ ID NO: 177 - protein, 102 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCP

SEQ ID NO: 178 - protein, 102 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCP

SEQ ID NO: 179 - protein, 101 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWP

SEQ ID NO: 180 - protein, 103 aa, Homo sapiens
ASTQSPSVFPLTRCCKNIPSNATSVTLGCLATGYFPEPVMVTCDTGSLNGTTMTLPATTLTLSGHYATISLLTVSGAWAKQMFTCRVAHTPSSTDWVDNKTFS

SEQ ID NO: 181 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 182 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 183 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 184 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 185 - protein, 104 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLP

SEQ ID NO: 186 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 187 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 188 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 189 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 190 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 191 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 192 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 193 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 194 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 195 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 196 - protein, 131 aa, Homo sapiens; identical to SEQ ID NO: 195

SEQ ID NO: 197 - protein, 117 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 198 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 199 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 201 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 202 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 204 - protein, 19 aa, Homo sapiens
VPSTPPTPSPSTPPTPSPS

SEQ ID NO: 205 - protein, 6 aa, Homo sapiens
VPPPPP

SEQ ID NO: 206 - protein, 58 aa, Homo sapiens
ESPKAQASSVPTAQPQAEGSLAKATTAPATTRNTGRGGEEKKKEKEKEEQEERETKTP

SEQ ID NO: 207 - protein, 15 aa, Homo sapiens
EPKSCDKTHTCPPCP

SEQ ID NO: 208 - protein, 12 aa, Homo sapiens
ERKCCVECPPCP

SEQ ID NO: 209 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 210 - protein, 12 aa, Homo sapiens
ESKYGPPCPSCP

SEQ ID NO: 211 - protein, 107 aa, Homo sapiens
GTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 212 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 213 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 214 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 215 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 216 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 217 - protein, 98 aa, Unknown
  Camel
ASTKAPSVYPLTARSGDTPGSTVAFGCLVWGYIPEPVTVTWNSGAVSSGIHTFPSVLMSLGLYSLSSLVTLPTSTSTGKTFISNVAHPASSTKVDKSV

SEQ ID NO: 218 - protein, 110 aa, Unknown
  Camel
APELLGGPSVFIFPPKPKDVLSISGRPEVTCVVVDVGQEDPEVSFNWYIDGVEVRTANTKPKEEQFNSTYRVVSVLTIQHQDWLTGKELKCKVNNKALPAPIERTISKAK

SEQ ID NO: 219 - protein, 110 aa, Unknown
  Camel
APELPGGPSVFVFPPKPKDVLSISGRPEVTCVVVDVGKEDPEVNFNWYIDGVEVRTANTKPKEEQFNSTYRVVSVLTIQHQDWLTGKEFKCKVNNKALPAPIERTISKPK

SEQ ID NO: 220 - protein, 109 aa, Unknown
  Camel
GQTREPQVYTLAPHREELAKDTVSITCLVIGFYPADINVEWQRNGRPESEGAYATTLPQLDNDGTYFLYSKLSVGKNTWQQGETFTCVVMHEALHNHSTQKSITQSSGK

SEQ ID NO: 221 - protein, 109 aa, Unknown
  Camel
GQTREPQVYTLAPHREELAKDTVSVTCLVKGFYPPDINVEWQRNRQPESEGAYATTLPQLDNDGTYFLYSKLSVGKNTWQRGETFTCVVMHEALHNHYTQKSITQSSGK

SEQ ID NO: 222 - protein, 35 aa, Unknown
  Camel
EPKIPQPQPKPQPQPQPQPKPQPKPEPECTCPKCP

SEQ ID NO: 223 - protein, 12 aa, Unknown
  Camel
GTNEVCKCPKCP

SEQ ID NO: 224 - protein, 91 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSP

SEQ ID NO: 225 - protein, 129 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 226 - protein, 129 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRA

SEQ ID NO: 227 - protein, 93 aa, Homo sapiens
RSQPHTKPSVFVMKNGTNVACLVKEFYPKDIRINLVSSKKITEFDPAIVISPSGKYNAVKLGKYEDSNSVTCSVQHDNKTVHSTDFEVKTDST

SEQ ID NO: 228 - protein, 110 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKT

SEQ ID NO: 229 - protein, 110 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKT

SEQ ID NO: 230 - protein, 110 aa, Homo sapiens; identical to SEQ ID NO: 229

SEQ ID NO: 231 - protein, 14 aa, Unknown
  Nurse Shark
SYEYGGGTVVTVNP

SEQ ID NO: 232 - protein, 12 aa, Unknown
  Nurse Shark
SFDEYGGGTVVT

SEQ ID NO: 233 - protein, 11 aa, Unknown
  Nurse Shark
SYEYGGGTVVT

SEQ ID NO: 234 - protein, 12 aa, Unknown
  Nurse Shark
NYAACGDGTAVT

SEQ ID NO: 235 - protein, 13 aa, Unknown
  Nurse Shark
GYWGQGTMVTVTT

SEQ ID NO: 236 - protein, 14 aa, Unknown
  Nurse Shark
ALDYWGQGTRVTVT

SEQ ID NO: 237 - protein, 12 aa, Unknown
  Nurse Shark
ACGSGTAVTVTP

SEQ ID NO: 238 - protein, 11 aa, Unknown
  Nurse Shark
YGGGTGVTVNP

SEQ ID NO: 239 - protein, 11 aa, Unknown
  Nurse Shark
YGGGTVVTVNP

SEQ ID NO: 240 - protein, 12 aa, Unknown
  Nurse Shark
ACGDGTFVTVNP

SEQ ID NO: 241 - protein, 11 aa, Unknown
  Nurse Shark
YGADTVVTVNP

SEQ ID NO: 242 - protein, 14 aa, Unknown
  Nurse Shark
YAACGAGTAVTVNP

SEQ ID NO: 243 - protein, 11 aa, Unknown
  Nurse Shark
YGSGTVLTVNP

SEQ ID NO: 244 - protein, 12 aa, Unknown
  Nurse Shark
ACGDGTAVTVNP

SEQ ID NO: 245 - protein, 11 aa, Unknown
  Nurse Shark
YGGGTVVTVNP

SEQ ID NO: 246 - protein, 15 aa, Unknown
  Nurse Shark
IYDECGDGTAVTVNP

SEQ ID NO: 247 - protein, 11 aa, Unknown
  Nurse Shark
CGDGTVVTVNP

SEQ ID NO: 248 - protein, 11 aa, Unknown
  Nurse Shark
CGGGTVVTVNP

SEQ ID NO: 249 - protein, 11 aa, Unknown
  Nurse Shark
YGGGTVVTVNP

SEQ ID NO: 250 - protein, 14 aa, Unknown
  Nurse Shark
YAAVGDGTAVTVNP

SEQ ID NO: 251 - protein, 11 aa, Unknown
  Nurse Shark
CGEGTAVTVNP

SEQ ID NO: 252 - protein, 13 aa, Unknown
  Nurse Shark
AACGHGTAVTVTS

SEQ ID NO: 253 - protein, 11 aa, Unknown
  Nurse Shark
YGGGTVVTVNP

SEQ ID NO: 254 - protein, 11 aa, Unknown
  Nurse Shark
CGDGTTVTVHP

SEQ ID NO: 255 - protein, 14 aa, Unknown
  Nurse Shark
YAACGPGTTVTVNP

SEQ ID NO: 256 - protein, 14 aa, Unknown
  Nurse Shark
YAACGHGTAVTVNA

SEQ ID NO: 257 - protein, 12 aa, Unknown
  Nurse Shark
ACGHGTAVTVNP

SEQ ID NO: 258 - protein, 11 aa, Unknown
  Nurse Shark
CGDGTAVTVNP

SEQ ID NO: 259 - protein, 14 aa, Unknown
  Nurse Shark
YAACGDATAVTVNP

SEQ ID NO: 260 - protein, 12 aa, Unknown
  Nurse Shark
ACGDGTVVTVSP

SEQ ID NO: 261 - protein, 11 aa, Unknown
  Nurse Shark
CGDGTAVTVNP

SEQ ID NO: 262 - protein, 14 aa, Unknown
  Nurse Shark
YAACGDGTAVTVNP

SEQ ID NO: 263 - protein, 11 aa, Unknown
  Nurse Shark
CGDNTAVTVNP

SEQ ID NO: 264 - protein, 12 aa, Unknown
  Nurse Shark
ACGHGTTVTVAP

SEQ ID NO: 265 - protein, 11 aa, Unknown
  Nurse Shark
CGDGTVLTVNP

SEQ ID NO: 266 - protein, 13 aa, Unknown
  Nurse Shark
AACGDGTAVTVNP

SEQ ID NO: 267 - protein, 12 aa, Unknown
  Nurse Shark
ACGLGTAVTVNP

SEQ ID NO: 268 - protein, 14 aa, Unknown
  Nurse Shark
YAACGDGTAVTVNP

SEQ ID NO: 269 - protein, 13 aa, Unknown
  Nurse Shark
AACGDGTAVTVNP

SEQ ID NO: 270 - protein, 11 aa, Unknown
  Nurse Shark
YGGGTVVTVNP

SEQ ID NO: 271 - protein, 12 aa, Unknown
  Nurse Shark
ACGDGTAVTVNP

SEQ ID NO: 272 - protein, 13 aa, Unknown
  Nurse Shark
AACGDGTAVTVNP

SEQ ID NO: 273 - protein, 11 aa, Unknown
  Nurse Shark
CGGGTDVTVNT

SEQ ID NO: 274 - protein, 13 aa, Unknown
  Nurse Shark
AACGDGTAVTVNP

SEQ ID NO: 275 - protein, 14 aa, Unknown
  Nurse Shark
YAACGDGTAVTVNP

SEQ ID NO: 276 - protein, 14 aa, Unknown
  Nurse Shark
YAACGDGTSVTVNP

SEQ ID NO: 277 - protein, 14 aa, Unknown
  Nurse Shark
YAACGDGTAVTVNP

SEQ ID NO: 278 - protein, 13 aa, Unknown
  Nurse Shark
AACGEGTAVTVNP

SEQ ID NO: 279 - protein, 14 aa, Unknown
  Nurse Shark
YAACGDGTTVTVKP

SEQ ID NO: 280 - protein, 14 aa, Unknown
  Nurse Shark
LDYWGDGTFLEVTS

SEQ ID NO: 281 - protein, 11 aa, Unknown
  Nurse Shark
CGDGTAVTVNP

SEQ ID NO: 282 - protein, 12 aa, Unknown
  Nurse Shark
ACGDGTAVTVNP

SEQ ID NO: 283 - protein, 13 aa, Unknown
  Nurse Shark
FAFGKGTKLRLSR

SEQ ID NO: 284 - protein, 13 aa, Unknown
  Nurse Shark
FAFGKGTKLRLSR

SEQ ID NO: 285 - protein, 13 aa, Unknown
  Nurse Shark
FVFGKGTKLRLSR

SEQ ID NO: 286 - protein, 13 aa, Unknown
  Nurse Shark
FAFGKGTKLRLSR

SEQ ID NO: 287 - protein, 13 aa, Unknown
  Nurse Shark
RAFGKGTKLRLSR

SEQ ID NO: 288 - protein, 13 aa, Unknown
  Nurse Shark
VVFGEGTKLRLSR

SEQ ID NO: 289 - protein, 20 aa, Unknown
  Mouse
LLKREDKATFATGGYEAEED

SEQ ID NO: 290 - protein, 20 aa, Unknown
  Mouse
QQGTGSKLSFGKGAKLTVSP

SEQ ID NO: 291 - protein, 20 aa, Unknown
  Mouse
NQGGSAKLIFGEGTKLTVSS

SEQ ID NO: 292 - protein, 20 aa, Unknown
  Mouse
ATGGNNKLTFGQGTVLSVIP

SEQ ID NO: 293 - protein, 21 aa, Unknown
  Mouse
NSGGSNYKLTFGKGTLLTVTP

SEQ ID NO: 294 - protein, 21 aa, Unknown
  Mouse
NTGANTGKLTFGHGTILRVHP

SEQ ID NO: 295 - protein, 20 aa, Unknown
  Mouse
ASSSFSKLVFGQGTSLSVVP

SEQ ID NO: 296 - protein, 19 aa, Unknown
  Mouse
NTGYQNFYFGKGTSLTVIP

SEQ ID NO: 297 - protein, 20 aa, Unknown
  Mouse
ANYGNEKITFGAGTKLTIKP

SEQ ID NO: 298 - protein, 18 aa, Unknown
  Mouse
HYANKMICGLGTILRVRP

SEQ ID NO: 299 - protein, 20 aa, Unknown
  Mouse
RRQQCRHAGFGDGDELGVST

SEQ ID NO: 300 - protein, 20 aa, Unknown
  Mouse
NTEGADRLTFGKGTQLIIQP

SEQ ID NO: 301 - protein, 20 aa, Unknown
  Mouse
VTGSGGKLTLGAGTRLQVNL

SEQ ID NO: 302 - protein, 18 aa, Unknown
  Mouse
NNNNAPRFGAGTKLSVKP

SEQ ID NO: 303 - protein, 21 aa, Unknown
  Mouse
NSGGSNAKLTFGKGTKLSVKS

SEQ ID NO: 304 - protein, 18 aa, Unknown
  Mouse
VSNTSSMLAEAPHYWSHP

SEQ ID NO: 305 - protein, 20 aa, Unknown
  Mouse
VNTGNYKYVFGAGTRLKVIA

SEQ ID NO: 306 - protein, 20 aa, Unknown
  Mouse
NNNAGAKLTFGGGTRLTVRP

SEQ ID NO: 307 - protein, 20 aa, Unknown
  Mouse
NVGDNSKLIWGLGTSLVVNP

SEQ ID NO: 308 - protein, 19 aa, Unknown
  Mouse
TGNTGKLIFGLGTTLQVQP

SEQ ID NO: 309 - protein, 21 aa, Unknown
  Mouse
QTGFASALTFGSGTKVIPCLP

SEQ ID NO: 310 - protein, 19 aa, Unknown
  Mouse
SSNTDKVVFGTGTRLQVSP

SEQ ID NO: 311 - protein, 18 aa, Unknown
  Mouse
DSNYQLIWGSGTKLIIKP

SEQ ID NO: 312 - protein, 21 aa, Unknown
  Mouse
NYGSSGNKLIFGIGTLLSVKP

SEQ ID NO: 313 - protein, 18 aa, Unknown
  Mouse
NSNNRIFFGDGTQLVVKP

SEQ ID NO: 314 - protein, 19 aa, Unknown
  Mouse
DTNAYKVIFGKGTHLHVLP

SEQ ID NO: 315 - protein, 19 aa, Unknown
  Mouse
NSGSRELVLGREARLSMIE

SEQ ID NO: 316 - protein, 21 aa, Unknown
  Mouse
LPGTGSNRLTFGKGTKFSLIP

SEQ ID NO: 317 - protein, 19 aa, Unknown
  Mouse
NTNTGKLTFGDGTVLTVKP

SEQ ID NO: 318 - protein, 18 aa, Unknown
  Mouse
RTKVSSVFGTWRRLLVKP

SEQ ID NO: 319 - protein, 20 aa, Unknown
  Mouse
ELASLGKLQFGTGTQVVVTP

SEQ ID NO: 320 - protein, 19 aa, Unknown
  Mouse
NYNQGKLIFGQGTKLSIKP

SEQ ID NO: 321 - protein, 19 aa, Unknown
  Mouse
SSGSWQLIFGSGTQLTVMP

SEQ ID NO: 322 - protein, 18 aa, Unknown
  Mouse
SNYNVLYFGSGTKLTVEP

SEQ ID NO: 323 - protein, 18 aa, Unknown
  Mouse
SGNYKLGVESVTMMSVRA

SEQ ID NO: 324 - protein, 20 aa, Unknown
  Mouse
IYRGFHKFSSGIESKHNVSP

SEQ ID NO: 325 - protein, 21 aa, Unknown
  Mouse
DRGSALGRLHFGAGTQLIVIP

SEQ ID NO: 326 - protein, 20 aa, Unknown
  Mouse
TNSAGNKLTFGIGTRVLVRP

SEQ ID NO: 327 - protein, 20 aa, Unknown
  Mouse
ATSSGQKLVFGQGTILKVYL

SEQ ID NO: 328 - protein, 19 aa, Unknown
  Mouse
YQGGRALIFGTGTTVSVSP

SEQ ID NO: 329 - protein, 18 aa, Unknown
  Mouse
NSGTYQRFGTGTKLQVVP

SEQ ID NO: 330 - protein, 19 aa, Unknown
  Mouse
GTGGYKVVFGSGTRLLVSP

SEQ ID NO: 331 - protein, 19 aa, Unknown
  Mouse
DSGYNKLTFGKGTVLLVSP

SEQ ID NO: 332 - protein, 19 aa, Unknown
  Mouse
RNMGYKLTFGTGTSLLVDP

SEQ ID NO: 333 - protein, 19 aa, Unknown
  Mouse
DYSNNRLTLGKGTQVVVLP

SEQ ID NO: 334 - protein, 20 aa, Unknown
  Mouse
TSGGNYKPTFGKGTSLVVHP

SEQ ID NO: 335 - protein, 20 aa, Unknown
  Mouse
GTQVVGQLTFGRGTRLQVYA

SEQ ID NO: 336 - protein, 20 aa, Unknown
  Mouse
LSGSFNKLTFGAGTRLLCAH

SEQ ID NO: 337 - protein, 21 aa, Unknown
  Mouse
EFSYSSKLIFGAETKLRNPPY

SEQ ID NO: 338 - protein, 21 aa, Unknown
  Mouse
NTGGLSGKLTFGEGTQVTVIS

SEQ ID NO: 339 - protein, 15 aa, Unknown
  Mouse
NTEVFFGKGTRLTVV

SEQ ID NO: 340 - protein, 15 aa, Unknown
  Mouse
NSDYTFGSGTRLLVI

SEQ ID NO: 341 - protein, 16 aa, Unknown
  Mouse
SGNTLYFGEGSRLIVV

SEQ ID NO: 342 - protein, 16 aa, Unknown
  Mouse
SNERLFFGHGTKLSVL

SEQ ID NO: 343 - protein, 16 aa, Unknown
  Mouse
NNQAPLFGEGTRLSVL

SEQ ID NO: 344 - protein, 17 aa, Unknown
  Mouse
SYNSPLYFAAGTRLTVT

SEQ ID NO: 345 - protein, 15 aa, Unknown
  Mouse
PVLDDHGLGKELRYK

SEQ ID NO: 346 - protein, 16 aa, Unknown
  Mouse
NYAEQFFGPGTRLTVL

SEQ ID NO: 347 - protein, 16 aa, Unknown
  Mouse
NTGQLYFGEGSKLTVL

SEQ ID NO: 348 - protein, 16 aa, Unknown
  Mouse
SAETLYFGSGTRLTVL

SEQ ID NO: 349 - protein, 16 aa, Unknown
  Mouse
SQNTLYFGAGTRLSVL

SEQ ID NO: 350 - protein, 16 aa, Unknown
  Mouse
NQDTQYFGPGTRLLVL

SEQ ID NO: 351 - protein, 15 aa, Unknown
  Mouse
SYEQYFGPGTRLTVL

SEQ ID NO: 352 - protein, 16 aa, Unknown
  Mouse
TDKLVFGQGTQVTVEP

SEQ ID NO: 353 - protein, 19 aa, Unknown
  Mouse
SWDTRQMFFGTGIELFVEP

SEQ ID NO: 354 - protein, 52 aa, Unknown
  Camelid
QVQLQDSGGGLVQAGGSLRLSCAVSGRTFSAHSVYTMGWFRQAPGKEREFVA

SEQ ID NO: 355 - protein, 54 aa, Unknown
  Camelid
QVQLQESGGGLVQPGGSLRLSCAASGRTFSDHSGYTYTIGWFRQAPGKEREFVA

SEQ ID NO: 356 - protein, 49 aa, Unknown
  Camelid
QVQLQESGGGLVQPGGSLRLSCATSGFDFSVSWMYWVRQAPGKGLEWVS

SEQ ID NO: 357 - protein, 49 aa, Unknown
  Camelid
QVQLQESGGGLVQPGGSLRLSCATSGFTFSDYWMYWVRQAPGKGLEWVS

SEQ ID NO: 358 - protein, 49 aa, Unknown
  Camelid
QVQLQESGGGLVQPGGSLRLSCAASGFEFENHWMYWVRQAPGKGLEWVS

SEQ ID NO: 359 - protein, 49 aa, Unknown
  Camelid
QVQLQESGGGLVQPGGSLRLSCAASGSIFRVNAMGWYRQVPGNQREFVA

SEQ ID NO: 360 - protein, 49 aa, Unknown
  Camelid
RIYWSSANTYYADSVKGRFTISRDNAKNTVDLLMNSLKPEDTAVYYCAA

SEQ ID NO: 361 - protein, 49 aa, Unknown
  Camelid
RIYWSSGNTYYADSVKGRFAISRDIAKNTVDLTMNNLEPEDTAVYYCAA

SEQ ID NO: 362 - protein, 49 aa, Unknown
  Camelid
EINTNGLITKYVDSVKGRFTISRDNAKNTLYLQMDSLIPEDTALYYCAR

SEQ ID NO: 363 - protein, 49 aa, Unknown
  Camelid
TVNTNGLITRYADSVKGRFTISRDNAKYTLYLQMNSLKSEDTAVYYCTK

SEQ ID NO: 364 - protein, 49 aa, Unknown; identical to SEQ ID NO: 363
  Camelid

SEQ ID NO: 365 - protein, 48 aa, Unknown
  Camelid
IITSGDNLNYADAVKGRFTISTDNVKKTVYLQMNVLKPEDTAVYYCNA

SEQ ID NO: 366 - protein, 35 aa, Unknown
  Camelid
RDGIPTSRTVGSYNYWGQGTQVTVSSEPKTPKPQP

SEQ ID NO: 367 - protein, 35 aa, Unknown
  Camelid
RDGIPTSRSVESYNYWGQGTQVTVSSEPKTPKPQP

SEQ ID NO: 368 - protein, 26 aa, Unknown
  Camelid
SPSGSFRGQGTQVTVSSEPKTPKPQP

SEQ ID NO: 369 - protein, 34 aa, Unknown
  Camelid
VVPPYSDDSRTNADWGQGTQVTVSSEPKTPKPQP

SEQ ID NO: 370 - protein, 34 aa, Unknown
  Camelid
VLPPYSDDSRTNADWGQGTQVTVSSEPKTPKPQP

SEQ ID NO: 371 - protein, 33 aa, Unknown
  Camelid
ILQTSRWSIPSNYWGQGTQVTVSSEPKTPKPQP

SEQ ID NO: 372 - protein, 50 aa, Unknown
  Camelid
QVQLQDSGGGLVQAGGSLRLSCAASGGTFSSIIMAWFRQAPGKEREFVGA

SEQ ID NO: 373 - protein, 50 aa, Unknown
  Camelid
QVQLQDSGGGLVQAGGSLRLSCGVSGLSFSGYTMGWFRQAPGKEREFAAA

SEQ ID NO: 374 - protein, 50 aa, Unknown
  Camelid
EVQLVESGGGLVQAGGSLRLSCAASGGTLSSYITGWFRQAPGKEREFVGA

SEQ ID NO: 375 - protein, 50 aa, Unknown
  Camelid
QVQLVESGGGLVQAGGSLRLSCAASEGTLSGYILGWFRQAPGKEREFVGA

SEQ ID NO: 376 - protein, 48 aa, Unknown
  Camelid
VSWSGGTTVYADSVLGRFEISRDSARKSVYLQMNSLKPEDTAVYYCAA

SEQ ID NO: 377 - protein, 48 aa, Unknown
  Camelid
IGWNSGTTEYRNSVKGRFTISRDNAKNTVYLQMNSLKPEDTAVYYCAA

SEQ ID NO: 378 - protein, 48 aa, Unknown
  Camelid
VSWSSSTIVYADSVEGRFTISRDNHQNTVYLQMDSLKPEDTAVYYCAA

SEQ ID NO: 379 - protein, 48 aa, Unknown
  Camelid
VSWSGGTIVYADSVKGRFEISRDNARNTVYLQMDSLKSEDTAVYYCAA

SEQ ID NO: 380 - protein, 35 aa, Unknown
  Camelid
RPYQKYNWASASYNVWGQGTQVTVSSEPKTPKPQP

SEQ ID NO: 381 - protein, 34 aa, Unknown
  Camelid
SPKYMTAYERSYDFWGQGTQVTVSSEPKTPKPQP

SEQ ID NO: 382 - protein, 26 aa, Unknown
  Camelid
RPYQKYNWASASYNVWGQGTQVTVSS

SEQ ID NO: 383 - protein, 26 aa, Unknown
  Camelid
RPYQRFNWASASYNVWGRGTQVTVSS

SEQ ID NO: 384 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 385 - protein, 123 aa, Unknown
  Camelid
QVQLQESGGGLVQPGGSLRLSCAASGFEFENHWMYWVRQAPGKGLEWVSTVNTNGLITRYADSVKGRFTISRDNAKYTLYLQMNSLKSEDTAVYYCTKVLPPYSDDSRTNADWGQGTQVTVSS

SEQ ID NO: 386 - protein, 4 aa, Unknown
  Conserved motif in antibody FR4
  VARIANT 2: Xaa = Any Amino Acid
GXGT

SEQ ID NO: 387 - protein, 6 aa, Unknown
  Conserved motif in antibody FR4
  VARIANT 2, 5: Xaa = Any Amino Acid
  VARIANT 6: Xaa = Val or Leu
GXGTXX

SEQ ID NO: 388 - protein, 4 aa, Unknown
  Conserved motif in antibody FR3
EDTA

SEQ ID NO: 389 - protein, 4 aa, Unknown
  Conserved motif in antibody FR3
VYYC

SEQ ID NO: 390 - protein, 8 aa, Unknown [sequence omitted]

SEQ ID NO: 391 - protein, 4 aa, Unknown
  Conserved motif in antibody constant region
  VARIANT 1: Xaa = Ser, Ala or Gly
  VARIANT 3: Xaa = Lys, Asp or Ser
XPXV

SEQ ID NO: 392 - protein, 5 aa, Unknown
  Conserved motif in antibody constant region
  VARIANT 1: Xaa = Ser, Ala or Gly
  VARIANT 3: Xaa = Lys, Asp or Ser
XPXVF

SEQ ID NO: 393 - protein, 5 aa, Unknown
  Conserved motif in antibody constant region
  VARIANT 5: Xaa = Ser, Arg or Thr
KVDKX

SEQ ID NO: 394 - protein, 3 aa, Unknown
  Conserved motif in antibody constant region
VTV

SEQ ID NO: 395 - protein, 6 aa, Unknown
  Conserved motif
  VARIANT 2, 5: Xaa = Any Amino Acid
GXGTXV

SEQ ID NO: 396 - protein, 6 aa, Unknown
  Conserved motif
  VARIANT 2, 5: Xaa = Any Amino Acid
GXGTXL

SEQ ID NO: 397 - protein, 4 aa, Unknown
  Conserved motif
PSVF

SEQ ID NO: 398 - protein, 4 aa, Unknown
  Conserved motif
SPKV

SEQ ID NO: 399 - protein, 4 aa, Unknown
  Conserved motif
SPDV

SEQ ID NO: 400 - protein, 4 aa, Unknown
  Conserved motif
SPSV

SEQ ID NO: 401 - protein, 4 aa, Unknown
  Conserved motif
APKV

SEQ ID NO: 402 - protein, 4 aa, Unknown
  Conserved motif
APDV

SEQ ID NO: 403 - protein, 4 aa, Unknown
  Conserved motif
APSV

SEQ ID NO: 404 - protein, 4 aa, Unknown
  Conserved motif
GPKV

SEQ ID NO: 405 - protein, 4 aa, Unknown
  Conserved motif
GPDV

SEQ ID NO: 406 - protein, 4 aa, Unknown
  Conserved motif
GPSV

SEQ ID NO: 407 - protein, 5 aa, Unknown
  Conserved motif
SPKVF

SEQ ID NO: 408 - protein, 5 aa, Unknown
  Conserved motif
SPDVF

SEQ ID NO: 409 - protein, 5 aa, Unknown
  Conserved motif
SPSVF

SEQ ID NO: 410 - protein, 5 aa, Unknown
  Conserved motif
APKVF

SEQ ID NO: 411 - protein, 5 aa, Unknown
  Conserved motif
APDVF

SEQ ID NO: 412 - protein, 5 aa, Unknown
  Conserved motif
APSVF

SEQ ID NO: 413 - protein, 5 aa, Unknown
  Conserved motif
GPKVF

SEQ ID NO: 414 - protein, 5 aa, Unknown
  Conserved motif
GPDVF

SEQ ID NO: 415 - protein, 5 aa, Unknown
  Conserved motif
GPSVF

SEQ ID NO: 416 - protein, 5 aa, Unknown
  Conserved motif
KVDKS

SEQ ID NO: 417 - protein, 5 aa, Unknown
  Conserved motif
KVDKR

SEQ ID NO: 418 - protein, 5 aa, Unknown
  Conserved motif
KVDKT

SEQ ID NO: 419 - protein, 4 aa, Unknown
  antibody FR
TVSS

SEQ ID NO: 420 - protein, 6 aa, Unknown
  antibody FR
  VARIANT 1: Xaa = Leu, Met or Thr
XVTVSS

SEQ ID NO: 421 - protein, 6 aa, Unknown
  antibody FR
LVTVSS

SEQ ID NO: 422 - protein, 6 aa, Unknown
  antibody FR
MVTVSS

SEQ ID NO: 423 - protein, 6 aa, Unknown
  antibody FR
TVTVSS

SEQ ID NO: 424 - protein, 5 aa, Unknown
  antibody FR
  VARIANT 1: Xaa = Lys or Arg
  VARIANT 2: Xaa = Val or Leu
  VARIANT 3: Xaa = Glu or Asp
XXXIK

SEQ ID NO: 425 - protein, 5 aa, Unknown
  antibody FR
  VARIANT 1: Xaa = Lys, Gln or Glu
  VARIANT 2: Xaa = Val or Leu
  VARIANT 3: Xaa = Thr or Ile
  VARIANT 4: Xaa = Val or Ile
XXXXL

SEQ ID NO: 426 - protein, 5 aa, Unknown
  antibody FR
KVEIK

SEQ ID NO: 427 - protein, 5 aa, Unknown
  antibody FR
KVDIK

SEQ ID NO: 428 - protein, 5 aa, Unknown
  antibody FR
KLEIK

SEQ ID NO: 429 - protein, 5 aa, Unknown
  antibody FR
KLDIK

SEQ ID NO: 430 - protein, 5 aa, Unknown
  antibody FR
RVEIK

SEQ ID NO: 431 - protein, 5 aa, Unknown
  antibody FR
RVDIK

SEQ ID NO: 432 - protein, 5 aa, Unknown
  antibody FR
RLEIK

SEQ ID NO: 433 - protein, 5 aa, Unknown
  antibody FR
RLDIK

SEQ ID NO: 434 - protein, 5 aa, Unknown
  antibody FR
KVTVL

SEQ ID NO: 435 - protein, 5 aa, Unknown
  antibody FR
KVTIL

SEQ ID NO: 436 - protein, 5 aa, Unknown
  antibody FR
KVIVL

SEQ ID NO: 437 - protein, 5 aa, Unknown
  antibody FR
KVIIL

SEQ ID NO: 438 - protein, 5 aa, Unknown
  antibody FR
KLTVL

SEQ ID NO: 439 - protein, 5 aa, Unknown
  antibody FR
KLTIL

SEQ ID NO: 440 - protein, 5 aa, Unknown
  antibody FR
KLIVL

SEQ ID NO: 441 - protein, 5 aa, Unknown
  antibody FR
KLIIL

SEQ ID NO: 442 - protein, 5 aa, Unknown
  antibody FR
QVTVL

SEQ ID NO: 443 - protein, 5 aa, Unknown
  antibody FR
QVTIL

SEQ ID NO: 444 - protein, 5 aa, Unknown
  antibody FR
QVIVL

SEQ ID NO: 445 - protein, 5 aa, Unknown
  antibody FR
QVIIL

SEQ ID NO: 446 - protein, 5 aa, Unknown
  antibody FR
QLTVL

SEQ ID NO: 447 - protein, 5 aa, Unknown
  antibody FR
QLTIL

SEQ ID NO: 448 - protein, 5 aa, Unknown
  antibody FR
QLIVL

SEQ ID NO: 449 - protein, 5 aa, Unknown
  antibody FR
QLIIL

SEQ ID NO: 450 - protein, 5 aa, Unknown
  antibody FR
EVTVL

SEQ ID NO: 451 - protein, 5 aa, Unknown
  antibody FR
EVTIL

SEQ ID NO: 452 - protein, 5 aa, Unknown
  antibody FR
EVIVL

SEQ ID NO: 453 - protein, 5 aa, Unknown
  antibody FR
EVIIL

SEQ ID NO: 454 - protein, 5 aa, Unknown
  antibody FR
ELTVL

SEQ ID NO: 455 - protein, 5 aa, Unknown
  antibody FR
ELTIL

SEQ ID NO: 456 - protein, 5 aa, Unknown
  antibody FR
ELIVL

SEQ ID NO: 457 - protein, 5 aa, Unknown
  antibody FR
ELIIL

SEQ ID NO: 458 - protein, 3 aa, Unknown
  antibody FR
  VARIANT 1: Xaa = Glu or Asp
XIK

SEQ ID NO: 459 - protein, 3 aa, Unknown
  antibody FR
  VARIANT 1: Xaa = Thr or Ile
  VARIANT 2: Xaa = Val or Ile
XXL

SEQ ID NO: 460 - protein, 3 aa, Unknown
  antibody FR
EIK

SEQ ID NO: 461 - protein, 3 aa, Unknown
  antibody FR
DIK

SEQ ID NO: 462 - protein, 3 aa, Unknown
  antibody FR
TVL

SEQ ID NO: 463 - protein, 3 aa, Unknown
  antibody FR
TIL

SEQ ID NO: 464 - protein, 3 aa, Unknown
  antibody FR
IVL

SEQ ID NO: 465 - protein, 3 aa, Unknown
  antibody FR
IIL

SEQ ID NO: 466 - protein, 4 aa, Unknown
  Lamda constant domain
QPKA

SEQ ID NO: 467 - protein, 3 aa, Unknown
  Kappa constant domain
TVA

SEQ ID NO: 468 - protein, 4 aa, Unknown
  Conserved motif
  VARIANT 1: Xaa = Ala or Gly
XPSV

SEQ ID NO: 469 - protein, 3 aa, Unknown
  CH1
STK

SEQ ID NO: 470 - protein, 5 aa, Unknown
  Kappa constant domain
  VARIANT 1: Xaa = Ala or Gly
XPSVF

SEQ ID NO: 471 - protein, 6 aa, Artificial Sequence
  Natural junction
KVEIKR

SEQ ID NO: 472 - protein, 10 aa, Unknown
  antibody FR4
GQGTKVTVSS

SEQ ID NO: 473 - protein, 3 aa, Unknown
  CH1
AST

SEQ ID NO: 474 - protein, 7 aa, Unknown
  CH1
ASTKGPS

SEQ ID NO: 475 - protein, 8 aa, Unknown
  CH1
ASTKGPSG

SEQ ID NO: 476 - protein, 9 aa, Unknown
  antibody FR4
  VARIANT 2: Xaa = Any Amino Acid
  VARIANT 5: Xaa = Lys, Gln or Glu
  VARIANT 6: Xaa = Val or Leu
  VARIANT 7: Xaa = Thr or Ile
GXGTXXXVL

SEQ ID NO: 477 - protein, 10 aa, Unknown
  antibody FR4
GQGTKVEIKR

SEQ ID NO: 478 - protein, 10 aa, Unknown
  antibody FR4
  VARIANT 2: Xaa = Any Amino Acid
  VARIANT 5: Xaa = Leu, Met or Thr
GXGTXVTVSS

SEQ ID NO: 479 - protein, 10 aa, Unknown
  antibody FR4
  VARIANT 2: Xaa = Any Amino Acid
GXGTLVTVSS

SEQ ID NO: 480 - protein, 10 aa, Unknown
  antibody FR4
  VARIANT 2: Xaa = Any Amino Acid
GXGTMVTVSS

SEQ ID NO: 481 - protein, 10 aa, Unknown
  antibody FR4
  VARIANT 2: Xaa = Any Amino Acid
GXGTTVTVSS

SEQ ID NO: 482 - protein, 5 aa, Unknown
  natural junction
KVTVL

SEQ ID NO: 483 - protein, 5 aa, Unknown
  natural junction
LVTVL

SEQ ID NO: 484 - protein, 6 aa, Unknown
  natural junction
LVTVSS

SEQ ID NO: 485 - protein, 10 aa, Unknown
  antibody FR4
  VARIANT 2: Xaa = any Amino Acid
  VARIANT 5: Xaa = Lys or Arg
  VARIANT 6: Xaa = Val or Leu
  VARIANT 7: Xaa = Glu or Asp
GXGTXXXIKR

SEQ ID NO: 486 - protein, 10 aa, Unknown
  antibody FR4
  VARIANT 2: Xaa = Any Amino Acid
GXGTKVEIKR

SEQ ID NO: 487 - protein, 10 aa, Unknown
  antibody FR4
  VARIANT 2: Xaa = Any Amino Acid
GXGTKLEIKR

SEQ ID NO: 488 - protein, 10 aa, Unknown
  antibody FR4
  VARIANT 2: Xaa = Any Amino Acid
GXGTKVDIKR

SEQ ID NO: 489 - protein, 10 aa, Unknown
  antibody FR4
  VARIANT 2: Xaa = Any Amino Acid
GXGTRKEIKR

SEQ ID NO: 490 - protein, 6 aa, Unknown
  Kappa constant domain
TVAAPS

SEQ ID NO: 491 - protein, 9 aa, Unknown
  Kappa constant domain
TVALAAPSG

SEQ ID NO: 492 - protein, 9 aa, Unknown
  antibody FR4
  VARIANT 2: Xaa = Any Amino Acid
  VARIANT 5: Xaa = Lys, Gln or Glu
  VARIANT 6: Xaa = Val or Leu
  VARIANT 7: Xaa = Thr or Ile
  VARIANT 8: Xaa = Val or Ile
GXGTXXXXL

SEQ ID NO: 493 - protein, 9 aa, Unknown
  antibody FR4
  VARIANT 2: Xaa = Any Amino Acid
GXGTKVTVL

SEQ ID NO: 494 - protein, 9 aa, Unknown
  antibody FR4
  VARIANT 2: Xaa = Any Amino Acid
GXGTKVTIL

SEQ ID NO: 495 - protein, 9 aa, Unknown
  antibody FR4
  VARIANT 2: Xaa = Any Amino Acid
GXGTKVIVL

SEQ ID NO: 496 - protein, 9 aa, Unknown
  antibody FR4
  VARIANT 2: Xaa = Any Amino Acid
GXGTKVIIL

SEQ ID NO: 497 - protein, 9 aa, Unknown
  antibody FR4
  VARIANT 2: Xaa = Any Amino Acid
GXGTKLTVL

SEQ ID NO: 498 - protein, 9 aa, Unknown
  antibody FR4
  VARIANT 2: Xaa = Any Amino Acid
GXGTKLTIL

SEQ ID NO: 499 - protein, 9 aa, Unknown
  antibody FR4
  VARIANT 2: Xaa = Any Amino Acid
GXGTKLIVL

SEQ ID NO: 500 - protein, 9 aa, Unknown
  antibody FR4
  VARIANT 2: Xaa = Any Amino Acid
GXGTKLIIL

SEQ ID NO: 501 - protein, 9 aa, Unknown
  antibody FR4
  VARIANT 2: Xaa = Any Amino Acid
GXGTQVTVL

SEQ ID NO: 502 - protein, 9 aa, Unknown
  antibody FR4
  VARIANT 2: Xaa = Any Amino Acid
GXGTQVTIL

SEQ ID NO: 503 - protein, 9 aa, Unknown
  antibody FR4
  VARIANT 2: Xaa = Any Amino Acid
GXGTQVIVL

SEQ ID NO: 504 - protein, 9 aa, Unknown
  antibody FR4
  VARIANT 2: Xaa = Any Amino Acid
GXGTQVIIL

SEQ ID NO: 505 - protein, 9 aa, Unknown
  antibody FR4
  VARIANT 2: Xaa = Any Amino Acid
GXGTQLTVL

SEQ ID NO: 506 - protein, 9 aa, Unknown
  antibody FR4
  VARIANT 2: Xaa = Any Amino Acid
GXGTQLTIL

SEQ ID NO: 507 - protein, 9 aa, Unknown
  antibody FR4
  VARIANT 2: Xaa = Any Amino Acid
GXGTQLIVL

SEQ ID NO: 508 - protein, 9 aa, Unknown
  antibody FR4
  VARIANT 2: Xaa = Any Amino Acid
GXGTQLIIL

SEQ ID NO: 509 - protein, 9 aa, Unknown
  antibody FR4
  VARIANT 2: Xaa = Any Amino Acid
GXGTEVTVL

SEQ ID NO: 510 - protein, 9 aa, Unknown
  antibody FR4
  VARIANT 2: Xaa = Any Amino Acid
GXGTEVTIL

SEQ ID NO: 511 - protein, 9 aa, Unknown
  antibody FR4
  VARIANT 2: Xaa = Any Amino Acid
GXGTEVIVL

SEQ ID NO: 512 - protein, 9 aa, Unknown
  antibody FR4
  VARIANT 2: Xaa = Any Amino Acid
GXGTEVIIL

SEQ ID NO: 513 - protein, 9 aa, Unknown
  antibody FR4
  VARIANT 2: Xaa = Any Amino Acid
GXGTELTVL

SEQ ID NO: 514 - protein, 9 aa, Unknown
  antibody FR4
  VARIANT 2: Xaa = Any Amino Acid
GXGTELTIL

SEQ ID NO: 515 - protein, 9 aa, Unkown
  antibody FR4
  VARIANT 2: Xaa = Any Amino Acid
GXGTELIVL

SEQ ID NO: 516 - protein, 9 aa, Unknown
  antibody FR4
  VARIANT 2: Xaa = Any Amino Acid
GXGTELIIL

SEQ ID NO: 517 - protein, 330 aa, Unknown [sequence omitted]

SEQ ID NO: 518 - protein, 106 aa, Unknown
  CK
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - protein, 106 aa, Unknown; identical to SEQ ID NO: 213
  CL2

SEQ ID NO: 520 - protein, 8 aa, Artificial Sequence
  Antibody hinge
THTCPPCP

SEQ ID NO: 521 - protein, 9 aa, Artificial Sequence
  Antibody hinge
GTHTCPPCP

SEQ ID NO: 522 - protein, 18 aa, Artificial Sequence
  Unnatural junction
TFGQGTKVEIKASTKGPS

SEQ ID NO: 523 - protein, 19 aa, Artificial Sequence
  Unnatural junction
TFGQGTKVEIKRASTKGPS

SEQ ID NO: 524 - protein, 19 aa, Artificial Sequence
  Natural junction
TFGQGTLVTVSSASTKGPS

SEQ ID NO: 525 - protein, 20 aa, Artificial Sequence
  Unnatural junction
FDYWGQGTLVTVSSTVAAPS

SEQ ID NO: 526 - protein, 21 aa, Artificial Sequence
  Unnatural junction
FDYWGQGTLVTVSSRTVAAPS

SEQ ID NO: 527 - protein, 20 aa, Artificial Sequence
  Natural junction
FDYWGQGTKVEIKRTVAAPS

SEQ ID NO: 528 - protein, 12 aa, Artificial Sequence
  Linker
KVEIKRTVAAPS

SEQ ID NO: 529 - protein, 9 aa, Artificial Sequence
  Linker
LVTVSSAST

SEQ ID NO: 530 - protein, 15 aa, Artificial Sequence
  Linker
LVTVSSGGGGSGGGS

SEQ ID NO: 531 - protein, 16 aa, Artificial Sequence
  Linker
GQGTKVEIKRTVAAPS

SEQ ID NO: 532 - protein, 16 aa, Artificial Sequence
  Linker
GQGTNVEINRTVAAPS

SEQ ID NO: 533 - protein, 16 aa, Artificial Sequence
  Linker
GQGTNVEINQTVAAPS

SEQ ID NO: 534 - protein, 16 aa, Artificial Sequence
  Linker
GQGTNVEIQRTVAAPS

SEQ ID NO: 535 - protein, 16 aa, Artificial Sequence
  Linker
GQGTLVTVSSTVAAPS

SEQ ID NO: 536 - protein, 6 aa, Artificial Sequence
  Linker
TVAAPS

SEQ ID NO: 537 - protein, 6 aa, Artificial Sequence
  Linker
SAKTTP

SEQ ID NO: 538 - protein, 10 aa, Artificial Sequence
  Linker
SAKTTPKLGG

SEQ ID NO: 539 - protein, 17 aa, Artificial Sequence
  Linker
AKTTPKLEEGEFSEARV

SEQ ID NO: 540 - protein, 9 aa, Artificial Sequence
  Linker
AKTTPKLEE

SEQ ID NO: 541 - protein, 6 aa, Homo sapiens
KVEIKR

SEQ ID NO: 542 - protein, 12 aa, Artificial Sequence
  Linker
LVTVSSASTGPS